(19) United States
(12) Patent Application Publication
(10) Pub. No.: US 2012/0022045 A1
(43) Pub. Date: Jan. 26, 2012

(54) BRIDGED COMPOUNDS AS HIV INTEGRASE INHIBITORS

(76) Inventors: Shankar Venkatraman, Lansdale, PA (US); John S. Wai, Harleysville, PA (US); Wayne Thompson, Lansdale, PA (US); Boyoung Kim, Lansdale, PA (US); Richard C.A. Isaacs, Harleysville, PA (US); H. Marie Loughran, Perkasie, PA (US); Dai-Shi Su, Dresher, PA (US); John Lim, Perkiomenville, PA (US); Mark W. Embrey, Harleysville, PA (US); Peter D. Williams, Harleysville, PA (US)

(21) Appl. No.: 13/146,595

(22) PCT Filed: Jan. 25, 2010
(86) PCT No.: PCT/US10/21920

§ 371 Date: Jul. 27, 2011

Related U.S. Application Data
(60) Provisional application No. 61/206,143, filed on Jan. 28, 2009.

(51) Int. Cl. A61K 31/55; A61P 31/18; A61K 31/519; C07D 471/08; C07D 487/08; C07D 491/18

(52) U.S. Cl. 514/214.02; 540/579; 540/578; 544/250; 514/267; 544/252

(57) ABSTRACT

Compounds of Formula I are inhibitors of HIV integrase and inhibitors of HIV replication: the asterisk * in Q denotes the point of attachment to the rest of the compound; and n, L1, L2, X¹, X², χ³, Y, Z, R¹, R² and R³ are defined herein. The N compounds are useful for the prophylaxis or treatment of infection by HIV and the prophylaxis, treatment, or delay in the onset or progression of AIDS. The compounds are employed against HIV infection and AIDS as compounds per se (or as hydrates or solvates thereof) or in the form of pharmaceutically acceptable salts. The compounds and their salts can be employed as ingredients in pharmaceutical compositions, optionally in combination with other antivirals, immunomodulators, antibiotics or vaccines.

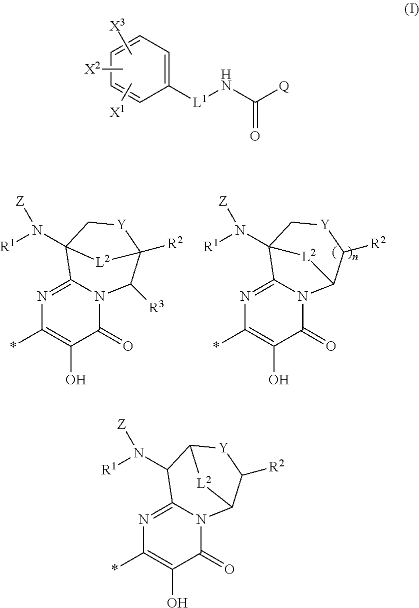

### FIELD OF THE INVENTION

The present invention is directed to certain bridged polyhydropyrimidoazepine carboxamides, bridged polyhydropyrimidooxazepine carboxamides, and related bridged compounds, and pharmaceutically acceptable salts thereof. These bridged compounds are inhibitors of the HIV integrase enzyme. The present invention is also directed to the use of the bridged compounds and their salts in the prophylaxis or treatment of infection by HIV and in the prophylaxis, treatment, or delay in the onset or progression of AIDS.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of the complex disease that includes progressive destruction of the immune system (acquired immune deficiency syndrome; AIDS) and degeneration of the central and peripheral nervous system. This virus was previously known as LAV, HTLV-III, or ARV. A common feature of retrovirus replication is the insertion by virally-encoded integrase of +proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277 (1985)]. Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells.

The following references are of interest as background:
Kinzel et al., Tet. Letters 2007, 48(37): pp. 6552-6555 discloses the synthesis of tetrahydropyridopyrimidones as a scaffold for HIV-1 integrase inhibitors.
Ferrara et al., Tet. Letters 2007, 48(37), pp. 8379-8382 discloses the synthesis of a hexahydropyrimido[1,2-a]azepine-2-carboxamide derivative useful as an HIV integrase inhibitor.
Muraglia et al., J. Med. Chem. 2008, 51: 861-874 discloses the design and synthesis of bicyclic pyrimidinones as potent and orally bioavailable HIV-1 integrase inhibitors.
US2004/229909 discloses certain compounds having integrase inhibitory activity.
U.S. Pat. No. 7,232,819 and US 2007/0083045 disclose certain 5,6-dihydroxypyrimidine-4-carboxamides as HIV integrase inhibitors.
U.S. Pat. No. 7,169,780, U.S. Pat. No. 7,217,713, and US 2007/0123524 disclose certain N-substituted 5-hydroxy-6-oxo-1,6-dihydropyrimidine-4-carboxamides as HIV integrase inhibitors.
U.S. Pat. No. 7,279,487 discloses certain hydroxynaphthyridinone carboxamides that are useful as HIV integrase inhibitors.
U.S. Pat. No. 7,135,467 and U.S. Pat. No. 7,037,908 disclose certain pyrimidine carboxamides that are useful as HIV integrase inhibitors.
U.S. Pat. No. 7,211,572 discloses certain nitrogenous condensed ring compounds that are HIV integrase inhibitors.
U.S. Pat. No. 7,414,045 discloses certain tetrahydro-4H-pyrido[1,2-a]pyrimidine carboxamides, hexahydropyrimido[1,2-a]azepine carboxamides, and related compounds that are useful as HIV integrase inhibitors.
WO 2006/103399 discloses certain tetrahydro-4H-pyrimidooxazepine carboxamides, tetrahydropyrazinopyrimidine carboxamides, hexahydropyrimidodiazepine carboxamides, and related compounds that are useful as HIV integrase inhibitors.
US 2007/0142635 discloses processes for preparing hexahydropyrimido[1,2-a]azepine-2-carboxylates and related compounds.
US 2007/0149556 discloses certain hydroxypyrimidinone derivatives having HIV integrase inhibitory activity.
Various pyrimidinone compounds useful as HIV integrase inhibitors are also disclosed in U.S. Pat. No. 7,115,601, U.S. Pat. No. 7,157,447, U.S. Pat. No. 7,173,022, U.S. Pat. No. 7,176,196, U.S. Pat. No. 7,192,948, U.S. Pat. No. 7,273,859, and U.S. Pat. No. 7,419,969.

US 2007/0111984 discloses a series of bicyclic pyrimidinone compounds useful as HIV integrase inhibitors.

US 2006/0276466, US 2007/0049606, US 2007/0111985, US 2007/0112190, US 2007/0281917, US 2008/0004265 each disclose a series of bicyclic pyrimidinone compounds useful as HIV integrase inhibitors.
U.S. Ser. No. 12/572,341, filed Oct. 2, 2009 (published as US 20______/______) discloses certain 2-{[(substituted benzyl)amino]carbonyl}-3-hydroxy-4-oxo-4,6,7,8,9,10-hexahydropyrimido[1,2-a]azepin-10-yl)-N,N′,N′-trialkylethanediamide compounds and certain 2-{[(substituted benzyl)amino]carbonyl}-3-hydroxy-4-oxo-6,7,9,10-tetrahydro-4H-pyrimido[1,2-d]-[1,4]oxazepin-10-yl)-N N′ N′-trialkylethanediamide compounds, which are useful as HIV integrase inhibitors.

### SUMMARY OF THE INVENTION

The present invention is directed to certain bridged polyhydropyrimidoazepine carboxamides, bridged polyhydropyrimidooxazepine carboxamides, and related bridged compounds. These bridged compounds (including hydrates and solvates thereof), optionally in the form of pharmaceutically acceptable salts, are useful in the inhibition of HIV integrase, the prophylaxis of infection by HIV, the treatment of infection by HIV and in the prophylaxis, treatment, and delay in the onset or progression of AIDS and/or ARC, either as compounds per se, or as pharmaceutical composition ingredients, whether or not in combination with other HIV/AIDS antivirals, anti-infectives, immunomodulators, antibiotics or vaccines. More particularly, the present invention includes compounds of Formula I and pharmaceutically acceptable salts thereof:

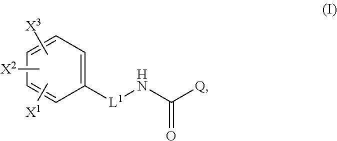

wherein:
Q is

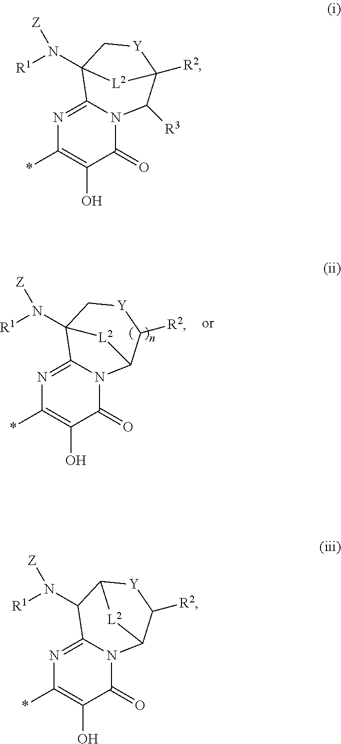

wherein the asterisk * denotes the point of attachment to the rest of the compound;
L¹ is CH₂, CH(CH₃), or C(CH₃)₂;
L² is C₁₋₄ alkylene;
X¹, X² and X³ are each independently selected from the group consisting of:
   (1) H,
   (2) C₁₋₆ alkyl,
   (3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B},
   (4) O—C₁₋₆ alkyl,
   (5) C₁₋₆ haloalkyl,
   (6) O—C₁₋₆ haloalkyl,
   (7) OH,
   (8) halogen,
   (9) CN,
   (10) NO₂,
   (11) N(R^{A})R^{B},
   (12) C(O)N(R^{A})R^{B},
   (13) C(O)R^{A},
   (14) C(O)—C₁₋₆ haloalkyl,
   (15) C(O)OR^{A},
   (16) OC(O)N(R^{A})R^{B},
   (17) SR^{A},
   (18) S(O)R^{A},
   (19) SO₂R^{A},
   (20) SO₂N(R^{A})R^{B},
   (21) SO₂N(R^{A})C(O)R^{B};
   (22) N(R^{A})SO₂R^{B},
   (23) N(R^{A})SO₂N(R^{A})R^{B},
   (24) N(R^{A})C(O)R^{B},
   (25) N(R^{A})C(O)N(R^{A})R^{B},
   (26) N(R^{A})C(O)C(O)N(R^{A})R^{B},
   (27) N(R^{A})CO₂R^{B}, and
   (28) HetB;
Y is CH₂, CH(CH₃), C(R^{A})(O-AryA), C(R^{A})(OR^{B}), O, S, SO₂, N(R^{A}), or C(O);
Z is:
   (1) C(O)N(R^{A})R^{B},
   (2) C(O)C(O)N(R^{A})R^{B},
   (3) SO₂N(R^{A})R^{B},
   (4) C(O)-HetA,
   (5) C(O)C(O)-HetA,
   (6) SO₂-HetA,
   (7) C(O)-HetB,
   (8) C(O)C(O)-HetB, or
   (9) SO₂-HetB;
R¹ is:
   (1) H,
   (2) C₁₋₆ alkyl,
   (3) C₁₋₆ haloalkyl,
   (4) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B}, or
   (5) C₁₋₆ alkyl substituted with AryC;
R² is:
   (1) H,
   (2) C₁₋₆ alkyl,
   (3) O—C₁₋₆ alkyl,
   (4) C₁₋₆ alkyl substituted with O—C₁₋₆ alkyl,
   (5) C(O)N(R^{C})R^{D}, or
   (6) SO₂N(R^{C})R^{D},
   (7) AryB, or
   (8) C₁₋₆ alkyl substituted with AryB;
R³ is:
   (1) H,
   (2) C₁₋₆ alkyl,
   (3) C₁₋₆ alkyl substituted with O—C₁₋₆ alkyl,
   (4) C(O)N(R^{C})R^{D},
   (5) C(O)C(O)N(R^{C})R^{D},
   (6) SO₂N(R^{C})R^{D},
   (7) AryB, or
   (8) C₁₋₆ alkyl substituted with AryB;
n is zero or 1;
each R^{A} is independently H or C₁₋₆ alkyl;
each R^{B} is independently H or C₁₋₆ alkyl;
each R^{C} is independently H or C₁₋₆ alkyl;
each R^{D} is independently H or C₁₋₆ alkyl;
   alternatively and independently each pair of R^{C} and R^{D} together with the N atom to which they are both attached form a 4- to 7-membered, saturated or unsaturated, non-aromatic monocyclic ring optionally containing 1 heteroatom in addition to the nitrogen attached to R^{C} and R^{D} selected from N, O, and S, where the S is optionally oxidized to S(O) or S(O)₂; wherein the monocyclic ring is optionally substituted with 1 or 2 substituents each of which is independently:
   (1) C₁₋₆ alkyl,
   (2) C₁₋₆ haloalkyl,
   (3) C₁₋₆ alkyl substituted with OH, O—C₁₋₆ alkyl, O—C₁₋₆ haloalkyl, N(R^{A})R^{B}, C(O)N(R^{A})R^{B}, C(O)R^{A}, CO₂R^{A}, or SO₂R^{A},
   (4) O—C₁₋₆ alkyl,
   (5) O—C₁₋₆ haloalkyl,
   (6) OH,
   (7) oxo,
   (8) halogen,
   (9) C(O)N(R^{A})R^{B},
   (10) C(O)R^{A},
   (11) C(O)—C₁₋₆ fluoroalkyl,
   (12) C(O)OR^{A}, or
   (13) S(O)₂R^{A};
AryA is phenyl or naphthyl, wherein the phenyl or naphthyl is optionally substituted with from 1 to 5 substituents each of which is independently any one of the substituents (2) to (28) as set forth above in the definition of X¹, X² and X³;
AryB is phenyl or naphthyl, wherein the phenyl or naphthyl is optionally substituted with from 1 to 5 substituents each of which is independently any one of the substituents (2) to (28) as set forth above in the definition of X¹, X² and X³;
AryC is phenyl or naphthyl, wherein the phenyl or naphthyl is optionally substituted with from 1 to 5 substituents each of which is independently any one of the substituents (2) to (28) as set forth above in the definition of X¹, X² and X³;
HetA is a 4- to 7-membered, saturated or unsaturated, non-aromatic heterocyclic ring containing at least one carbon atom and from 1 to 4 heteroatoms independently selected from N, O and S, where each S is optionally oxidized to S(O) or S(O)₂, wherein the heterocyclic ring is optionally substituted with from 1 to 4 substituents, each of which is independently:
   (1) halogen,
   (2) C₁₋₆ alkyl,
   (3) C₁₋₆ haloalkyl,
   (4) O—C₁₋₆ alkyl,
   (5) O—C₁₋₆ haloalkyl,
   (6) oxo,
   (7) C(O)N(R^{A})R^{B},
   (8) C(O)C(O)N(R^{A})R^{B},
   (9) C(O)R^{A},
   (10) CO₂R^{A},
   (11) SR^{A},
   (12) S(O)R^{A},
   (13) SO₂R^{A}, or
   (14) SO₂N(R^{A})R^{B}; and
each HetB is independently a 5- or 6-membered heteroaromatic ring containing from 1 to 4 heteroatoms independently selected from N, O and S, wherein the heteroaromatic ring is optionally substituted with from 1 to 4 substituents each of which is independently:
   (1) C₁₋₆ alkyl,
   (2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B},
   (3) O—C₁₋₆ alkyl,
   (4) C₁₋₆ haloalkyl,
   (5) O—C₁₋₆ haloalkyl,
   (6) OH,
   (7) halogen,
   (8) CN,
   (9) NO₂,
   (10) N(R^{A})R^{B},
   (11) C(O)N(R^{A})R^{B},
   (12) C(O)R^{A},
   (13) C(O)—C₁₋₆ haloalkyl,
   (14) C(O)OR^{A},
   (15) OC(O)N(R^{A})R^{B},
   (16) SR^{A},
   (17) S(O)R^{A},
   (18) SO₂R^{A},
   (19) SO₂N(R^{A})R^{B},
   (20) N(R^{A})SO₂R^{B},
   (21) N(R^{A})SO₂N(R^{A})R^{B},
   (22) N(R^{A})C(O)R^{B},
   (23) N(R^{A})C(O)N(R^{A})R^{B},
   (24) N(R^{A})C(O)C(O)N(R^{A})R^{B}, or
   (25) N(R^{A})CO₂R^{B}.

The present invention also includes pharmaceutical compositions containing a compound of Formula I or a pharmaceutically acceptable salt thereof. The present invention further includes methods involving compounds of Formula I for the treatment of AIDS, the delay in the onset or progression of AIDS, the prophylaxis of AIDS, the prophylaxis of infection by HIV, and the treatment of infection by HIV.

Other embodiments, aspects and features of the present invention are either further described in or will be apparent from the ensuing description, examples and appended claims.

### DETAILED DESCRIPTION OF THE INVENTION

The present invention includes compounds of Formula I above (including hydrates and solvates thereof), and pharmaceutically acceptable salts thereof. These compounds are effective inhibitors of wild-type HIV integrase (e.g., HIV-1) and mutant strains thereof, as demonstrated by the results shown in Examples 31 to 33 below.

A first embodiment of the present invention (alternatively referred to herein as “Embodiment E1”) is a compound of Formula I, or a pharmaceutically acceptable salt thereof, wherein Q is

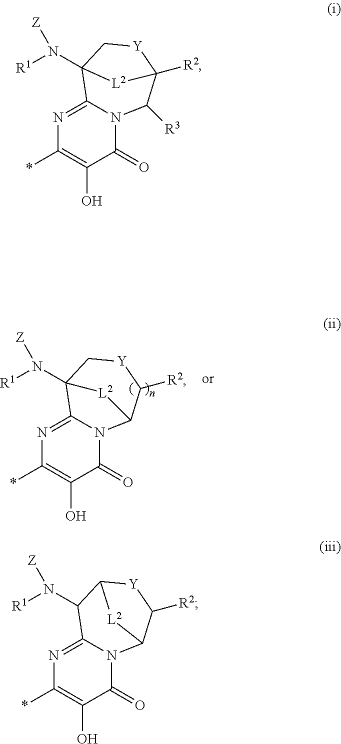

and all of the other variables are as originally defined (i.e., as defined in the Summary of the Invention).

A second embodiment of the present invention (Embodiment E2) is a compound of Formula II (alternatively and more simply referred to as “Compound II”), or a pharmaceutically acceptable salt thereof:

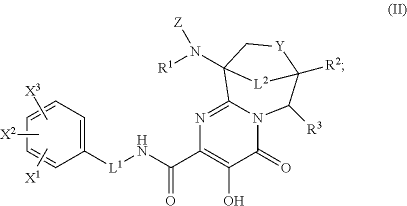

wherein all of the variables are as originally defined.

A third embodiment of the present invention (Embodiment E3) is a compound of Formula III (or Compound III), or a pharmaceutically acceptable salt thereof:

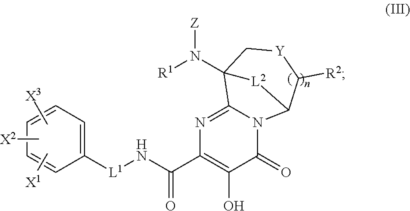

wherein all of the variables are as originally defined.

A fourth embodiment of the present invention (Embodiment E4) is a compound of Formula III-A (or Compound III-A), or a pharmaceutically acceptable salt thereof:

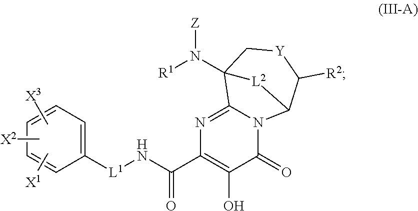

wherein all of the variables are as originally defined.

A fifth embodiment of the present invention (Embodiment E5) is a compound of Formula IV, or a pharmaceutically acceptable salt thereof:

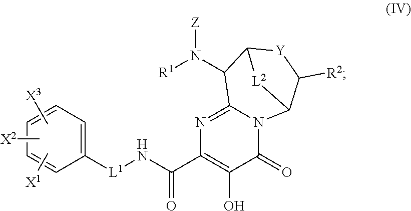

wherein all of the variables are as originally defined.

A sixth embodiment of the present invention (Embodiment E6) is a compound of Formula I or Formula II or Formula III or Formula III-A or Formula IV, or a pharmaceutically acceptable salt thereof, wherein L¹ is CH₂; and all other variables are as originally defined.

A seventh embodiment of the present invention (Embodiment E7) is a compound of Formula I or Formula II or Formula III or Formula IIIA or Formula IV, or a pharmaceutically acceptable salt thereof, wherein L² is CH₂, C(CH₃), C(CH₃)₂, CH₂CH₂, or CH₂CH₂CH₂; and all other variables are as originally defined or as defined in any of the preceding embodiments.

An eighth embodiment of the present invention (Embodiment E8) is a compound of Formula I or Formula II or Formula III or Formula III-A or Formula IV, or a pharmaceutically acceptable salt thereof, wherein L² is CH₂, CH₂CH₂, or CH₂CH₂CH₂; and all other variables are as originally defined or as defined in any of the preceding embodiments.

A ninth embodiment of the present invention (Embodiment E9) is a compound of Formula I or Formula II or Formula III or Formula III-A or Formula IV, or a pharmaceutically acceptable salt thereof, wherein L² is CH₂ or CH₂CH₂; and all other variables are as originally defined or as defined in any of the preceding embodiments. In an aspect of this embodiment, L² is CH₂. In another aspect of this embodiment, L² is CH₂CH₂.

A tenth embodiment of the present invention (Embodiment E10) is a compound of Formula I or Formula II or Formula III or Formula III-A or Formula IV, or a pharmaceutically acceptable salt thereof, wherein X¹, X² and X³ are each independently selected from the group consisting of H, halogen, CN, NO₂, C₁₋₄ alkyl, C₁₋₄ haloalkyl, OH, O—C₁₋₄ alkyl, O—C₁₋₄ haloalkyl, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, SO₂N(R^{A})C(O)R^{B}, N(R^{A})SO₂R^{B}, N(R^{A})SO₂N(R^{A})R^{B}, N(R^{A})C(O)R^{B}, and N(R^{A})C(O)C(O)N(R^{A})R^{B}; and provided that at least one of X¹, X² and X³ is other than H; and all other variables are as originally defined or as defined in any of the preceding embodiments.

An eleventh embodiment of the present invention (Embodiment E11) is a compound of Formula I or Formula II or Formula III or Formula III-A or Formula IV, or a pharmaceutically acceptable salt thereof, wherein:
X¹ and X² are each independently selected from the group consisting of H, Cl, Br, F, CN, C₁₋₃ alkyl, CF₃, OH, O—C₁₋₃ alkyl, OCF₃, NH₂, N(H)—C₁₋₃ alkyl, N(C₁₋₃ alkyl)₂, C(O)NH₂, C(O)N(H)—C₁₋₃ alkyl, C(O)N(C₁₋₃ alkyl)₂, CH(O), C(O)—C₁₋₃ alkyl, CO₂H, CO₂—C₁₋₃ alkyl, SO₂H and SO₂—C₁₋₃ alkyl; and provided that at least one of X¹ and X² is other than H;
X³ is H; and all other variables are as originally defined or as defined in any of the preceding embodiments.

A twelfth embodiment of the present invention (Embodiment E12) is a compound of Formula I or Formula II or Formula III or Formula III-A or Formula IV, or a pharmaceutically acceptable salt thereof, wherein:
X¹ and X² are each independently selected from the group consisting of H, Cl, Br, F, CN, CH₃, CF₃, OH, OCH₃, OCF₃, NH₂, N(H)CH₃, N(CH₃)₂, C(O)NH₂, C(O)N(H)CH₃, C(O)N(CH₃)₂, CH(O), C(O)CH₃, CO₂H, CO₂CH₃, SO₂H and SO₂CH₃; and provided that at least one of X¹ and X² is other than H;
X³ is H; and all other variables are as originally defined or as defined in any of the preceding embodiments.

A thirteenth embodiment of the present invention (Embodiment E13) is a compound of Formula I or Formula II or Formula III or Formula III-A or Formula IV, or a pharmaceutically acceptable salt thereof, wherein:
X¹ and X² are each independently selected from the group consisting of H, Cl, Br, F, CN, CH₃, CF₃, OH, OCH₃, OCF₃, NH₂, N(H)CH₃, N(CH₃)₂, C(O)NH₂, C(O)N(H)CH₃, C(O)N(CH₃)₂, CH(O), C(O)CH₃, CO₂H, CO₂CH₃, SO₂H and SO₂CH₃; and provided that
   (i) at least one of X¹ and X² is other than H;
   (ii) X¹ is in the para position on the phenyl ring; and
   (iii) X² is in the meta position on the phenyl ring;
X³ is H; and all other variables are as originally defined or as defined in any of the preceding embodiments.

A fourteenth embodiment of the present invention (Embodiment E14) is a compound of Formula I or Formula II or Formula III or Formula III-A or Formula IV, or a pharmaceutically acceptable salt thereof, wherein X¹ is F; X² is H or CH₃; and X³ is H; and all other variables are as originally defined or as defined in any of the preceding embodiments. In an aspect of this embodiment, X¹ is F, and X² is H. In a feature of this aspect, F is in the para position on the phenyl ring. In another aspect of this embodiment, X¹ is F, and X² is CH₃. In a feature of this aspect, F is in the para position and CH₃ is in the meta position on the phenyl ring.

A fifteenth embodiment of the present invention (Embodiment E15) is a compound of Formula I or Formula II or Formula III or Formula III-A or Formula IV, or a pharmaceutically acceptable salt thereof, wherein Y is CH₂, CH(CH₃), C(H)(O-phenyl), C(H)(OCH₃), O, S, SO₂, NH, N(CH₃), or C(O); and all other variables are as originally defined or as defined in any of the preceding embodiments.

A sixteenth embodiment of the present invention (Embodiment E16) is a compound of Formula I or Formula II or Formula III or Formula III-A or Formula IV, or a pharmaceutically acceptable salt thereof, wherein Y is CH₂ or O; and all other variables are as originally defined or as defined in any of the preceding embodiments.

A seventeenth embodiment of the present invention (Embodiment E17) is a compound of Formula I or Formula II or Formula III or Formula III-A or Formula IV, or a pharmaceutically acceptable salt thereof, wherein Y is CH₂; and all other variables are as originally defined or as defined in any of the preceding embodiments.

An eighteenth embodiment of the present invention (Embodiment E18) is a compound of Formula I or Formula II or Formula III or Formula III-A or Formula IV, or a pharmaceutically acceptable salt thereof, wherein Y is O; and all other variables are as originally defined or as defined in any of the preceding embodiments.

A nineteenth embodiment of the present invention (Embodiment E19) is a compound of Formula I or Formula II or Formula III or Formula III-A or Formula IV, or a pharmaceutically acceptable salt thereof, wherein Z is:

(1) C(O)N(R^{A})R^{B},

(2) C(O)C(O)N(R^{A})R^{B},

(3) C(O)-HetA,

(4) C(O)C(O)-HetA,

(5) C(O)-HetB, or

(6) C(O)C(O)-HetB;

and all other variables are as originally defined or as defined in any of the preceding embodiments.

A twentieth embodiment of the present invention (Embodiment E20) is a compound of Formula I or Formula II or Formula III or Formula III-A or Formula IV, or a pharmaceutically acceptable salt thereof, wherein Z is:

(1) C(O)N(C₁₋₃ alkyl)₂,

(2) C(O)C(O)NH(C₁₋₃ alkyl),

(3) C(O)C(O)N(C₁₋₃ alkyl)₂,

(4) C(O)-HetA,

(5) C(O)C(O)-HetA,

(6) C(O)-HetB, or

(7) C(O)C(O)-HetB;

and all other variables are as originally defined or as defined in any of the preceding embodiments.

A twenty-first embodiment of the present invention (Embodiment E21) is a compound of Formula I or Formula II or Formula III or Formula III-A or Formula IV, or a pharmaceutically acceptable salt thereof, wherein Z is:

(1) C(O)N(C₁₋₃ alkyl)₂,

(2) C(O)C(O)N(C₁₋₃ alkyl)₂,

(3) C(O)-HetA,

(4) C(O)C(O)-HetA,

(5) C(O)-HetB, or

(6) C(O)C(O)-HetB;

and all other variables are as originally defined or as defined in any of the preceding embodiments.

A twenty-second embodiment of the present invention (Embodiment E22) is a compound of Formula I or Formula II or Formula III or Formula III-A or Formula IV, or a pharmaceutically acceptable salt thereof, wherein Z is C(O)N(CH₃)₂, C(O)C(O)NH(CH₃), C(O)C(O)N(CH₃)₂,

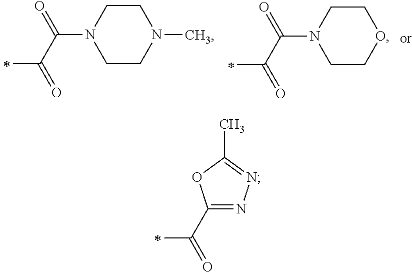

and all other variables are as originally defined or as defined in any of the preceding embodiments.

A twenty-third embodiment of the present invention (Embodiment E23) is a compound of Formula I or Formula II or Formula III or Formula III-A or Formula IV, or a pharmaceutically acceptable salt thereof, wherein Z is C(O)N(CH₃)₂, C(O)C(O)N(CH₃)₂,

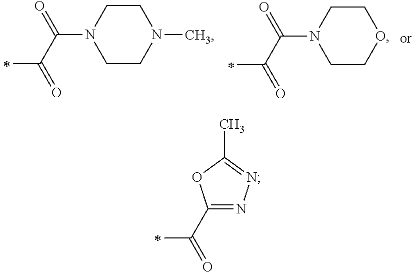

and all other variables are as originally defined or as defined in any of the preceding embodiments.

A twenty-fourth embodiment of the present invention (Embodiment E24) is a compound of Formula I or Formula II or Formula III or Formula III-A or Formula IV, or a pharmaceutically acceptable salt thereof, wherein R¹ is H or C₁₋₄ alkyl; and all other variables are as originally defined or as defined in any of the preceding embodiments.

A twenty-fifth embodiment of the present invention (Embodiment E25) is a compound of Formula I or Formula II or Formula III or Formula IIIA or Formula IV, or a pharmaceutically acceptable salt thereof, wherein R¹ is H or C₁₋₃ alkyl; and all other variables are as originally defined or as defined in any of the preceding embodiments.

A twenty-sixth embodiment of the present invention (Embodiment E26) is a compound of Formula I or Formula II or Formula III or Formula III-A or Formula IV, or a pharmaceutically acceptable salt thereof, wherein R¹ is C₁₋₃ alkyl; and all other variables are as originally defined or as defined in any of the preceding embodiments.

A twenty-seventh embodiment of the present invention (Embodiment E27) is a compound of Formula I or Formula II or Formula III or Formula III-A or Formula IV, or a pharmaceutically acceptable salt thereof, wherein R¹ is H, CH₃, CH₂CH₃, or CH₂CH₂CH₃; and all other variables are as originally defined or as defined in any of the preceding embodiments.

A twenty-eighth embodiment of the present invention (Embodiment E28) is a compound of Formula I or Formula II or Formula III or Formula IIIA or Formula IV, or a pharmaceutically acceptable salt thereof, wherein R¹ is H, CH₃, or CH₂CH₃; and all other variables are as originally defined or as defined in any of the preceding embodiments.

A twenty-ninth embodiment of the present invention (Embodiment E29) is a compound of Formula I or Formula II or Formula III or Formula III-A or Formula IV, or a pharmaceutically acceptable salt thereof, wherein R¹ is CH₃ or CH₂CH₃; and all other variables are as originally defined or as defined in any of the preceding embodiments.

A thirtieth embodiment of the present invention (Embodiment E30) is a compound of Formula I or Formula II or Formula III or Formula III-A or Formula IV, or a pharmaceutically acceptable salt thereof, wherein R¹ is CH₂CH₃; and all other variables are as originally defined or as defined in any of the preceding embodiments.

A thirty-first embodiment of the present invention (Embodiment E31) is a compound of Formula I or Formula II or Formula III or Formula III-A or Formula IV, or a pharmaceutically acceptable salt thereof; wherein R¹ is CH₃; and all other variables are as originally defined or as defined in any of the preceding embodiments.

A thirty-second embodiment of the present invention (Embodiment E32) is a compound of Formula I or Formula II or Formula III or Formula III-A or Formula IV, or a pharmaceutically acceptable salt thereof; wherein R² is:

(1) H,

(2) C₁₋₄ alkyl,

(3) O—C₁₋₄ alkyl,

(4) C₁₋₄ alkyl substituted with O—C₁₋₆ alkyl,

(5) C(O)N(R^{C})R^{D},

(6) SO₂N(R^{C})R^{D},

(7) AryB, or

(8) C₁₋₄ alkyl substituted with AryB;

and all other variables are as originally defined or as defined in any of the preceding embodiments.

A thirty-third embodiment of the present invention (Embodiment E33) is a compound of Formula I or Formula II or Formula III or Formula IIIA or Formula IV, or a pharmaceutically acceptable salt thereof, wherein R² is:

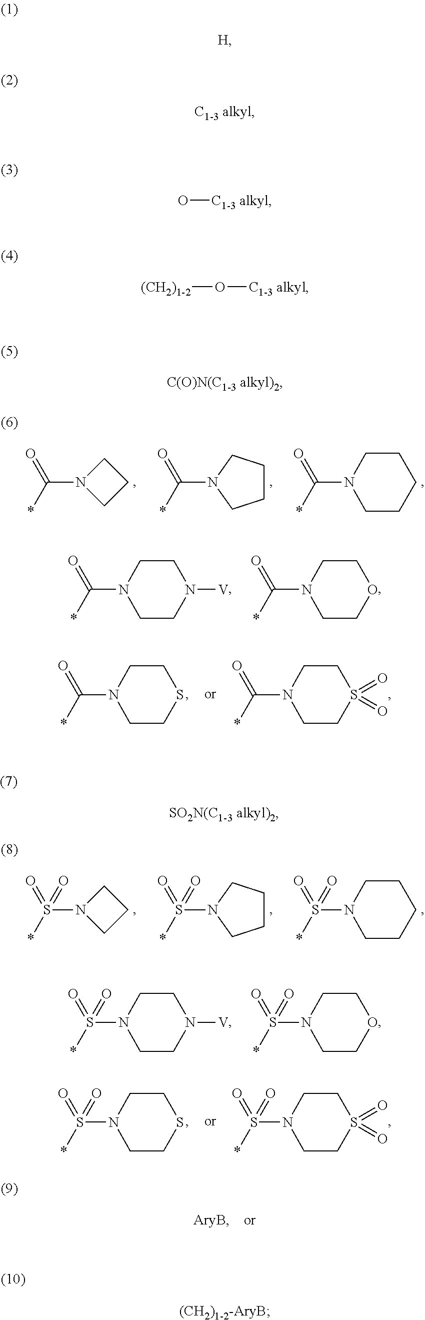

each V is independently H, C₁₋₃ alkyl, C(O)—C₁₋₃ alkyl, C(O)—O—C₁₋₃ alkyl, or S(O)₂—C₁₋₃ alkyl; and all other variables are as originally defined or as defined in any of the preceding embodiments.

A thirty-fourth embodiment of the present invention (Embodiment E34) is a compound of Formula I or Formula II or Formula III or Formula IIIA or Formula IV, or a pharmaceutically acceptable salt thereof, wherein R² is H, CH₃, CH₂CH₃, OCH₃, CH₂OCH₃, phenyl, or benzyl; wherein the phenyl or the phenyl moiety in benzyl is optionally substituted with 1 or 2 substituents each of which is independently Cl, Br, F, CH₃, CF₃, OCH₃, OCF₃, C(O)NH₂, C(O)N(H)CH₃, C(O)N(CH₃)₂, C(O)CH₃, CO₂CH₃, or SO₂CH₃; and all other variables are as originally defined or as defined in any of the preceding embodiments.

A thirty-fifth embodiment of the present invention (Embodiment E35) is a compound of Formula I or Formula II or Formula III or Formula III-A or Formula IV, or a pharmaceutically acceptable salt thereof, wherein R² is H, CH₃, CH₂CH₃, OCH₃, CH₂OCH₃, phenyl, or benzyl; and all other variables are as originally defined or as defined in any of the preceding embodiments.

A thirty-sixth embodiment of the present invention (Embodiment E36) is a compound of Formula I or Formula II or Formula III or Formula III-A or Formula IV, or a pharmaceutically acceptable salt thereof, wherein R² is H, CH₃, CH₂C₁₋₁₃, OCH₃, or CH₂OCH₃; and all other variables are as originally defined or as defined in any of the preceding embodiments.

A thirty-seventh embodiment of the present invention (Embodiment E37) is a compound of Formula I or Formula II or Formula III or Formula III-A or Formula IV, or a pharmaceutically acceptable salt thereof, wherein R² is H, CH₃, CH₂CH₃, OCH₃ or OH; and all other variables are as originally defined or as defined in any of the preceding embodiments.

A thirty-eighth embodiment of the present invention (Embodiment E38) is a compound of Formula I or Formula II or Formula III or Formula III-A or Formula IV, or a pharmaceutically acceptable salt thereof, wherein R² is H or CH₃; and all other variables are as originally defined or as defined in any of the preceding embodiments.

A thirty-ninth embodiment of the present invention (Embodiment E39) is a compound of Formula I or Formula II or Formula III or Formula III-A or Formula IV, or a pharmaceutically acceptable salt thereof, wherein R² is H; and all other variables are as originally defined or as defined in any of the preceding embodiments.

A fortieth embodiment of the present invention (Embodiment E40) is a compound of Formula I or Formula II, or a pharmaceutically acceptable salt thereof; wherein R³ is:

(1) H,

(2) C₁₋₄ alkyl,

(3) C₁₋₄ alkyl substituted with O—C₁₋₄ alkyl,

(4) C(O)N(R^{C})R^{D},

(5) C(O)C(O)N(R^{C})R^{D},

(6) SO₂N(R^{C})R^{D},

(7) AryB, or

(8) C₁₋₄ alkyl substituted with AryB;

and all other variables are as originally defined or as defined in any of the preceding embodiments.

A forty-first embodiment of the present invention (Embodiment E41) is a compound of Formula I or Formula II, or a pharmaceutically acceptable salt thereof, wherein R³ is H, C₁₋₃ alkyl, AryB, or (CH₂)₁₋₂-AryB; and all other variables are as originally defined or as defined in any of the preceding embodiments.

A forty-second embodiment of the present invention (Embodiment E42) is a compound of Formula I or Formula II, or a pharmaceutically acceptable salt thereof, wherein R³ is H, CH₃, CH₂CH₃, phenyl, or benzyl; wherein the phenyl or the phenyl moiety in benzyl is optionally substituted with 1 or 2 substituents each of which is independently Cl, Br, F, CH₃, CF₃, OCH₃, OCF₃, C(O)NH₂, C(O)N(H)CH₃, C(O)N(CH₃)₂, C(O)CH₃, CO₂CH₃, or SO₂CH₃; and all other variables are as originally defined or as defined in any of the preceding embodiments.

A forty-third embodiment of the present invention (Embodiment E43) is a compound of Formula I or Formula II, or a pharmaceutically acceptable salt thereof, wherein R³ is H, CH₃, CH₂CH₃, phenyl, or benzyl; and all other variables are as originally defined or as defined in any of the preceding embodiments.

A forty-fourth embodiment of the present invention (Embodiment E44) is a compound of Formula I or Formula II, or a pharmaceutically acceptable salt thereof, wherein R³ is H, CH₃, or CH₂CH₃; and all other variables are as originally defined or as defined in any of the preceding embodiments.

A forty-fifth embodiment of the present invention (Embodiment E45) is a compound of Formula I or Formula II, or a pharmaceutically acceptable salt thereof, wherein R³ is H or CH₃; and all other variables are as originally defined or as defined in any of the preceding embodiments.

A forty-sixth embodiment of the present invention (Embodiment E46) is a compound of Formula I or Formula II or Formula III or Formula III-A or Formula IV, or a pharmaceutically acceptable salt thereof, wherein R^{A} and R^{B} are each independently H or C₁₋₄ alkyl; and all other variables are as originally defined or as defined in any of the preceding embodiments.

A forty-seventh embodiment of the present invention (Embodiment E47) is a compound of Formula I or Formula II or Formula III or Formula III-A or Formula IV, or a pharmaceutically acceptable salt thereof, wherein R^{A} and R^{B} are each independently H or C₁₋₃ alkyl; and all other variables are as originally defined or as defined in any of the preceding embodiments.

A forty-eighth embodiment of the present invention (Embodiment E48) is a compound of Formula I or Formula II or Formula III or Formula III-A or Formula IV, or a pharmaceutically acceptable salt thereof, wherein R^{A} and R^{B} are each independently H or CH₃; and all other variables are as originally defined or as defined in any of the preceding embodiments.

A forty-ninth embodiment of the present invention (Embodiment E49) is a compound of Formula I or Formula II or Formula III or Formula III-A or Formula IV, or a pharmaceutically acceptable salt thereof, wherein R^{C} and R^{D} are each independently H or C₁₋₄ alkyl; or alternatively and independently each pair of R^{C} and R^{D} together with the N atom to which they are both attached form a 4- to 7-membered, saturated monocyclic ring optionally containing 1 heteroatom in addition to the nitrogen attached to R^{C} and R^{D} selected from N, O, and S, where the S is optionally oxidized to S(O) or S(O)₂; wherein the monocyclic ring is optionally substituted with 1 or 2 substituents each of which is independently:

(1) C₁₋₄ alkyl,

(2) C₁₋₄ fluoroalkyl,

(3) O—C₁₋₄ alkyl,

(4) O—C₁₋₄ fluoroalkyl,

(5) oxo,

(6) C(O)R^{A},

(7) CO₂R^{A}, or

(8) SO₂R^{A};

and all other variables are as originally defined or as defined in any of the preceding embodiments.

A fiftieth embodiment of the present invention (Embodiment E50) is a compound of Formula I or Formula II or Formula III or Formula III-A or Formula IV, or a pharmaceutically acceptable salt thereof, wherein R^{C} and R^{D} are each independently H or C₁₋₃ alkyl; or alternatively and independently each pair of R^{C} and R^{D} together with the N atom to which they are both attached form:

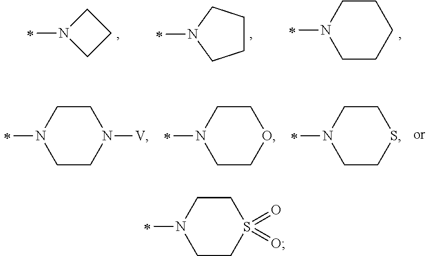

each V is independently H, C₁₋₃ alkyl, C(O)—C₁₋₃ alkyl, C(O)—O—C₁₋₃ alkyl, or S(O)₂—C₁₋₃ alkyl; and all other variables are as originally defined or as defined in any of the preceding embodiments.

A fifty-first embodiment of the present invention (Embodiment E51) is a compound of Formula I or Formula II or Formula III or Formula III-A or Formula IV, or a pharmaceutically acceptable salt thereof, wherein R^{C} and R^{D} are each independently H or CH₃; or alternatively and independently each pair of R^{C} and R^{D} together with the N atom to which they are both attached form:

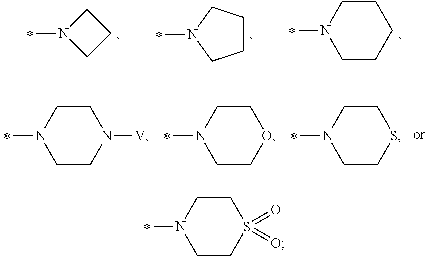

each V is independently H, CH₃, C(O)CH₃, C(O)OCH₃, or S(O)₂CH₃; and all other variables are as originally defined or as defined in any of the preceding embodiments.

A fifty-second embodiment of the present invention (Embodiment E52) is a compound of Formula I or Formula II or Formula III or Formula III-A or Formula IV, or a pharmaceutically acceptable salt thereof, wherein R^{C} and R^{D} are each independently H or C₁₋₃ alkyl; and all other variables are as originally defined or as defined in any of the preceding embodiments.

A fifty-third embodiment of the present invention (Embodiment E53) is a compound of Formula I or Formula II or Formula III or Formula III-A or Formula IV, or a pharmaceutically acceptable salt thereof, wherein R^{C} and R^{D} are each independently H or CH₃; and all other variables are as originally defined or as defined in any of the preceding embodiments.

A fifty-fourth embodiment of the present invention (Embodiment E54) is a compound of Formula I or Formula II or Formula III or Formula III-A or Formula IV, or a pharmaceutically acceptable salt thereof, wherein one, two or all three of AryA, AryB, and AryC are independently phenyl optionally substituted with from 1 to 3 substituents each of which is independently:

(1) C₁₋₄ alkyl,

(2) OH,

(3) O—C₁₋₄ alkyl,

(4) C₁₋₄ haloalkyl,

(5) O—C₁₋₄ haloalkyl,

(6) halogen,

(7) CN,

(8) N(R^{A})R^{B},

(9) C(O)N(R^{A})R^{B},

(10) C(O)R^{A},

(11) C(O)OR^{A},

(12) SR^{A},

(13) S(O)R^{A},

(14) SO₂R^{A},

(15) SO₂N(R^{A})R^{B},

(16) SO₂N(R^{A})C(O)R^{B},

(17) N(R^{A})SO₂R^{B},

(18) N(R^{A})SO₂N(R^{A})R^{B},

(19) N(R^{A})C(O)R^{B}, or

(20) N(R^{A})C(O)C(O)N(R^{A})R^{B};

and all other variables are as originally defined or as defined in any of the preceding embodiments.

A fifty-fifth embodiment of the present invention (Embodiment E55) is a compound of Formula I or Formula II or Formula III or Formula III-A or Formula IV, or a pharmaceutically acceptable salt thereof, wherein one, two or all three of AryA, AryB, and AryC are independently phenyl optionally substituted with from 1 to 3 substituents each of which is independently:

(1) C₁₋₃ alkyl,

(2) O—C₁₋₃ alkyl,

(3) CF₃,

(4) OCF₃,

(5) Cl,

(6) Br,

(7) F,

(8) CN,

(9) C(O)NH₂,

(10) C(O)N(H)—C₁₋₃ alkyl,

(11) C(O)N(—C₁₋₃ alkyl)₂,

(12) C(O)—C₁₋₃ alkyl,

(13) C(O)O—C₁₋₃ alkyl, or

(14) SO₂—C₁₋₃ alkyl;

and all other variables are as originally defined or as defined in any of the preceding embodiments.

A fifty-sixth embodiment of the present invention (Embodiment E56) is a compound of Formula I or Formula II or Formula III or Formula III-A or Formula IV, or a pharmaceutically acceptable salt thereof, wherein one, two or all three of AryA, AryB, and AryC are independently phenyl optionally substituted with from 1 to 3 substituents each of which is independently:

(1) CH₃,

(2) OCH₃,

(3) CF₃,

(4) OCF₃,

(5) Cl,

(6) Br,

(7) F,

(8) CN,

(9) C(O)NH₂,

(10) C(O)N(H)CH₃,

(11) C(O)N(CH₃)₂

(12) C(O)CH₃,

(13) C(O)OCH₃, or

(14) SO₂CH₃;

and all other variables are as originally defined or as defined in any of the preceding embodiments.

A fifty-seventh embodiment of the present invention (Embodiment E57) is a compound of Formula I or Formula II or Formula III or Formula III-A or Formula IV, or a pharmaceutically acceptable salt thereof, wherein one, two or all three of AryA, AryB, and AryC are independently phenyl optionally substituted with from 1 to 3 substituents each of which is independently CH₃, OCH₃, CF₃, OCF₃, Cl, Br, or F; and all other variables are as originally defined or as defined in any of the preceding embodiments.

A fifty-eighth embodiment of the present invention (Embodiment E58) is a compound of Formula I or Formula II or Formula III or Formula III-A or Formula IV, or a pharmaceutically acceptable salt thereof, wherein HetA is a 4- to 7-membered, saturated heterocyclic ring containing an N atom and optionally containing an additional heteroatom selected from N, O and S, wherein (i) the heterocyclic ring is attached to the rest of the compound via an N atom, (ii) the optional S atom is optionally oxidized to S(O) or S(O)₂, and (iii) the heterocyclic ring is optionally substituted with from 1 to 3 substituents, each of which is independently:

(1) C₁₋₄ alkyl,

(2) C₁₋₄ fluoroalkyl,

(3) O—C₁₋₄ alkyl,

(4) O—C₁₋₄ fluoroalkyl,

(5) oxo,

(6) C(O)R^{A},

(7) CO₂R^{A}, or

(8) SO₂R^{A};

and all other variables are as originally defined or as defined in any of the preceding embodiments.

A fifty-ninth embodiment of the present invention (Embodiment E59) is a compound of Formula I or Formula II or Formula III or Formula III-A or Formula IV, or a pharmaceutically acceptable salt thereof, wherein HetA is a saturated heterocyclic ring selected from the grout) consisting of:

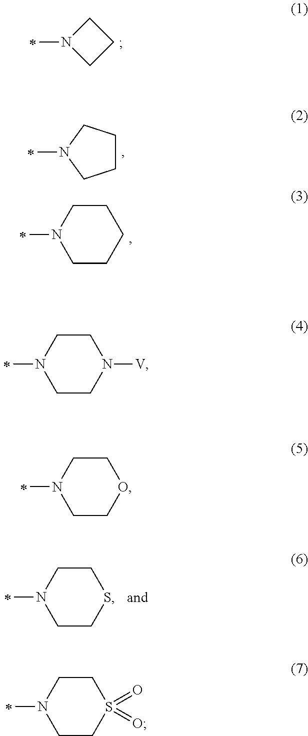

V is independently H, C₁₋₃ alkyl, C(O)—C₁₋₃ alkyl, C(O)—O—C₁₋₃ alkyl, or S(O)₂—C₁₋₃ alkyl; and all other variables are as originally defined or as defined in any of the preceding embodiments. In an aspect of this embodiment, V is independently H, CH₃, C(O)CH₃, C(O)OCH₃, or S(O)₂CH₃. In another aspect of this embodiment, V is CH₃, C(O)CH₃, C(O)OCH₃, or S(O)₂CH₃. In still another aspect of this embodiment, V is CH₃.

A sixtieth embodiment of the present invention (Embodiment E60) is a compound of Formula I or Formula II or Formula III or Formula III-A or Formula IV, or a pharmaceutically acceptable salt thereof, wherein HetB is a 5- or 6-membered heteroaromatic ring containing a total of from 1 to 4 heteroatoms independently selected from 1 to 4 N atoms, zero or 1 O atom, and zero or 1 S atom, wherein the heteroaromatic ring is optionally substituted with from 1 to 3 substituents each of which is independently:

(1) C₁₋₄ alkyl,

(2) C₁₋₄ fluoroalkyl,

(3) O—C₁₋₄ alkyl,

(4) O—C₁₋₄ fluoroalkyl,

(5) OH,

(6) C(O)R^{A},

(7) CO₂R^{A}, or

(8) SO₂R^{A};

and all other variables are as originally defined or as defined in any of the preceding embodiments.

A sixty-first embodiment of the present invention (Embodiment E61) is a compound of Formula I or Formula II or Formula III or Formula III-A or Formula IV, or a pharmaceutically acceptable salt thereof, wherein HetB is a heteroaromatic ring selected from the group consisting of pyrrolyl, pyrazolyl, imidazolyl, pyridinyl, pyrimidinyl, pyrazinyl, oxazolyl, isoxazolyl, thiazolyl, isothiazolyl, oxadiazolyl, and thiadiazolyl, wherein the heteroaromatic ring is optionally substituted with from 1 to 2 substituents each of which is independently a C₁₋₄ alkyl; and all other variables are as originally defined or as defined in any of the preceding embodiments.

A first class of compounds of the present invention (alternatively referred to herein as Class C1) includes compounds of Formula I and pharmaceutically acceptable salts thereof, wherein:
Q is as originally defined (see Summary of the Invention);
n is zero or 1;
L¹ is CH₂;
L² is CH₂, C(CH₃), C(CH₃)₂, CH₂CH₂, or CH₂CH₂CH₂;
X¹, X² and X³ are each independently selected from the group consisting of H, halogen, CN, NO₂, C₁₋₄ alkyl, C₁₋₄ haloalkyl, OH, O—C₁₋₄ alkyl, O—C₁₋₄ haloalkyl, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, SO₂N(R^{A})C(O)R^{B}; N(R^{A})SO₂R^{B}, N(R^{A})SO₂N(R^{A})R^{B}, N(R^{A})C(O)R^{B}, and N(R^{A})C(O)C(O)N(R^{A})R^{B}; and provided that at least one of X¹, X² and X³ is other than H;
Y is CH₂ or O;
Z is: (1) C(O)N(R^{A})R^{B}, (2) C(O)C(O)N(R^{A})R^{B}, (3) C(O)-HetA, (4) C(O)C(O)-HetA, (5) C(O)-HetB, or (6) C(O)C(O)-HetB;
R¹ is H or C₁₋₄ alkyl;
R² is: (1) H, (2) C₁₋₄ alkyl, (3) O—C₁₋₄ alkyl, (4) C₁₋₄ alkyl substituted with O—C₁₋₆ alkyl, (5) C(O)N(R^{C})R^{D}, (6) SO₂N(R^{C})R^{D}, (7) AryB, or (8) C₁₋₄ alkyl substituted with AryB;
R³ is: (1) H, (2) C₁₋₄ alkyl, (3) C₁₋₄ alkyl substituted with O—C₁₋₄ alkyl, (4) C(O)N(R^{C})R^{D}, (5) C(O)C(O)N(R^{C})R^{D}, (6) SO₂N(R^{C})R^{D}, (7) AryB, or (8) C₁₋₄ alkyl substituted with AryB;
each R^{A} is independently H or C₁₋₄ alkyl;
each R^{B} is independently H or C₁₋₄ alkyl;
each R^{C} is independently H or C₁₋₄ alkyl;
each R^{D} is independently H or C₁₋₄ alkyl;
or alternatively and independently each pair of R^{C} and R^{D} together with the N atom to which they are both attached form a 4- to 7-membered, saturated monocyclic ring optionally containing 1 heteroatom in addition to the nitrogen attached to R^{C} and R^{D} selected from N, O, and S, where the S is optionally oxidized to S(O) or S(O)₂; wherein the monocyclic ring is optionally substituted with 1 or 2 substituents each of which is independently: (1) C₁₋₄ alkyl, (2) C₁₋₄ fluoroalkyl, (3) O—C₁₋₄ alkyl, (4) O—C₁₋₄ fluoroalkyl, (5) oxo, (6) C(O)R^{A}, (7) CO₂R^{A}, or (8) SO₂R^{A};
Ary B is as defined in Embodiment E54;
HetA is as defined in Embodiment E58; and
HetB is as defined in Embodiment E60.

A first sub-class of the first class (alternatively referred to herein as “Sub-class C1-S1”) includes compounds and pharmaceutically acceptable salts thereof in which Q is defined as in Embodiment E1 (i.e., n=1)

and all other variables are as originally defined in Class C1.

A second sub-class of the first class (alternatively referred to herein as “Sub-class C1-S2”) includes compounds of Formula II and pharmaceutically acceptable salts thereof, wherein all of the variables are as originally defined in Class C1.

A third sub-class of the first class (Sub-class C1-S3) includes compounds of Formula III and pharmaceutically acceptable salts thereof, wherein all of the variables are as originally defined in Class C1.

A fourth sub-class of the first class (Sub-class C1-S4) includes compounds of Formula III-A and pharmaceutically acceptable salts thereof, wherein all of the variables are as originally defined in Class C1.

A fifth sub-class of the first class (Sub-class C1-S5) includes compounds of Formula IV and pharmaceutically acceptable salts thereof, wherein all of the variables are as originally defined in Class Cl.

A second class of compounds of the present invention (Class C2) includes compounds of Formula I and pharmaceutically acceptable salts thereof, wherein:
Q is as originally defined;
L¹ is CH₂;
L² is CH₂, C(CH₃), C(CH₃)₂, CH₂CH₂, or CH₂CH₂CH₂;
X¹ and X² are each independently selected from the group consisting of H, Cl, Br, F, CN, C₁₋₃ alkyl, CF₃, OH, O—C₁₋₃ alkyl, OCF₃, NH₂, N(H)—C₁₋₃ alkyl, N(C₁₋₃ alkyl)₂, C(O)NH₂, C(O)N(H)—C₁₋₃ alkyl, C(O)N(C₁₋₃ alkyl)₂, CH(O), C(O)—C₁₋₃ alkyl, CO₂H, CO₂—C₁₋₃ alkyl, SO₂H and SO₂—C₁₋₃ alkyl; and provided that at least one of X¹ and X² is other than H;
X³ is H;
Y is CH₂ or O;
Z is: (1) C(O)N(C₁₋₃ alkyl)₂, (2) C(O)C(O)NH(C₁₋₃ alkyl), (3) C(O)C(O)N(C₁₋₃ alkyl)₂, (4) C(O)-HetA, (5) C(O)C(O)-HetA, (6) C(O)-HetB, or (7) C(O)C(O)-HetB;
R¹ is H or C₁₋₃ alkyl;
R² is: (1) H, (2) C₁₋₃ alkyl, (3) O—C₁₋₃ alkyl, (4) (CH₂)₁₋₂—O—C₁₋₃ alkyl, (5) C(O)N(C₁₋₃ alkyl)₂,

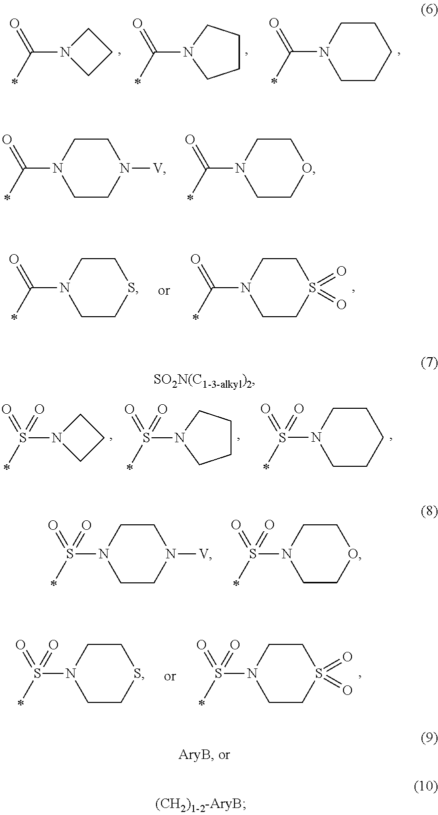

R³ is H, C₁₋₃ alkyl, AryB, or (CH₂)₁₋₂-AryB; AryB is phenyl optionally substituted with from 1 to 3 substituents each of which is independently: (1) C₁₋₃ alkyl, (2) O—C₁₋₃ alkyl, (3) CF₃, (4) OCF₃, (5) Cl, (6) Br, (7) F, (8) CN, (9) C(O)NH₂, (10) C(O)N(H)—C₁₋₃ alkyl, (11) C(O)N(—C₁₋₃ alkyl)₂, (12) C(O)—C₁₋₃ alkyl, (13) C(O)O—C₁₋₃ alkyl, or (14) SO₂—C₁₋₃ alkyl;
HetA is a saturated heterocyclic ring selected from the group consisting of:

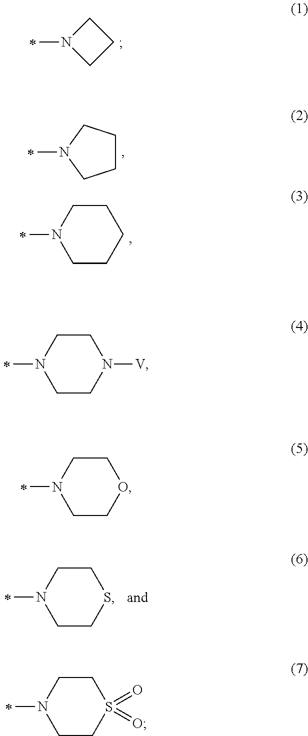

each V is independently H, C₁₋₃ alkyl, C(O)—C₁₋₃ alkyl, C(O)—O—C₁₋₃ alkyl, or S(O)₂—C₁₋₃ alkyl; and
HetB is a heteroaromatic ring selected from the group consisting of pyrrolyl, pyrazolyl, imidazolyl, pyridinyl, pyrimidinyl, pyrazinyl, oxazolyl, isoxazolyl, thiazolyl, isothiazolyl, oxadiazolyl, and thiadiazolyl, wherein the heteroaromatic ring is optionally substituted with from 1 to 2 substituents each of which is independently a C₁₋₄ alkyl.

A first sub-class of the second class (Sub-class C2-S1) includes compounds and pharmaceutically acceptable salts thereof in which Z is: (1) C(O)N(C₁₋₃ alkyl)₂, (2) C(O)C(O)N(C₁₋₃ alkyl)₂, (3) C(O)-HetA, (4) C(O)C(O)-HetA, (5) C(O)-HetB, or (6) C(O)C(O)-HetB; and all other variables are as originally defined in Class C2.

A second sub-class of the second class (Sub-class C2-S2) includes compounds and pharmaceutically acceptable salts thereof in which Q is defined as in Embodiment E1 and all other variables are as originally defined in Class C2. In an aspect of this sub-class, Q is defined as in Embodiment E1 and all other variables are as defined in Sub-class C2-S1.

A third sub-class of the second class (Sub-class C2-S3) includes compounds of Formula II and pharmaceutically acceptable salts thereof, wherein all of the variables are as originally defined in Class C2. In an aspect of this sub-class, all of the variables are as defined in Sub-class C2-S1.

A fourth sub-class of the second class (Sub-class C2-S4) includes compounds of Formula III and pharmaceutically acceptable salts thereof, wherein all of the variables are as originally defined in Class C2. In an aspect of this sub-class, all of the variables are as defined in Sub-class C2-S1.

A fifth sub-class of the second class (Sub-class C2-S5) includes compounds of Formula III-A and pharmaceutically acceptable salts thereof, wherein all of the variables are as originally defined in Class C2. In an aspect of this sub-class, all of the variables are as defined in Sub-class C2-S1.

A sixth sub-class of the second class (Sub-class C2-S6) includes compounds of Formula IV and pharmaceutically acceptable salts thereof, wherein all of the variables are as originally defined in Class C2. In an aspect of this sub-class, all of the variables are as defined in Sub-class C2-S1.

A third class of compounds of the present invention (Class C3) includes compounds of Formula I and pharmaceutically acceptable salts thereof, wherein:
Q is as originally defined;
L¹ is CH₂;
L² is CH₂, C(CH₃), C(CH₃)₂, CH₂CH₂, or CH₂CH₂CH₂;
X¹ and X² are each independently selected from the group consisting of H, CI, Br, F, CN, CH₃, CF₃, OH, OCH₃, OCF₃, NH₂, N(H)CH₃, N(CH₃)₂, C(O)NH₂, C(O)N(H)CH₃, C(O)N(CH₃)₂, CH(O), C(O)CH₃, CO₂H, CO₂CH₃, SO₂H and SO₂CH₃; and provided that at least one of X¹ and X² is other than H;
X³ is H;
Y is CH₂ or O;
Z is C(O)N(CH₃)₂, C(O)C(O)NH(CH₃), C(O)C(O)N(CH₃)₂,

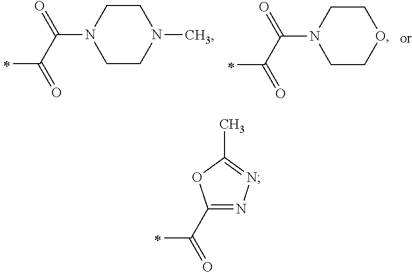

R¹ is H, CH₃, CH₂CH₃, CH₂CH₂CH₃, or CH(CH₃)₂;
R² is H, CH₃, CH₂CH₃, OCH₃, CH₂OCH₃, phenyl, or benzyl; wherein the phenyl or the phenyl moiety in benzyl is optionally substituted with 1 or 2 substituents each of which is independently Cl, Br, F, CH₃, CF₃, OCH₃, OCF₃, C(O)NH₂, C(O)N(H)CH₃, C(O)N(CH₃)₂, C(O)CH₃, CO₂CH₃, or SO₂CH₃; and
R³ is H, CH₃, CH₂CH₃, phenyl, or benzyl; wherein the phenyl or the phenyl moiety in benzyl is optionally substituted with 1 or 2 substituents each of which is independently Cl, Br, F, CH₃, CF₃, OCH₃, OCF₃, CN, C(O)NH₂, C(O)N(H)CH₃, C(O)N(CH₃)₂, C(O)CH₃, CO₂CH₃, or SO₂CH₃.

A first sub-class of the third class (Sub-class C3-S1) includes compounds of Formula I and pharmaceutically acceptable salts thereof, wherein:
Z is C(O)N(CH₃)₂, C(O)C(O)N(CH₃)₂,

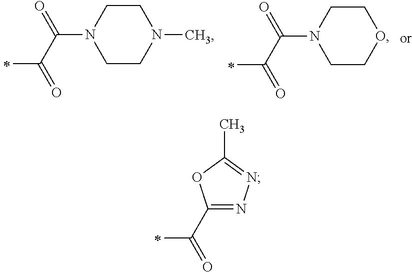

R¹ is H, CH₃, or CH₂CH₃; and all other variables are as originally defined in Class C3.

A second sub-class of the third class (Sub-class C3-S2) includes compounds and pharmaceutically acceptable salts thereof in which Q is defined as in Embodiment E1 and all other variables are as originally defined in Class C3. In an aspect of this sub-class, Q is defined as in Embodiment E1 and all other variables are as defined in Sub-class C3-S1.

A third sub-class of the third class (Sub-class C3-S3) includes compounds of Formula II and pharmaceutically acceptable salts thereof, wherein all of the variables are as originally defined in Class C3. In an aspect of this sub-class, all of the variables are as defined in Sub-class C3-S1.

A fourth sub-class of the third class (Sub-class C3-S4) includes compounds of Formula III and pharmaceutically acceptable salts thereof, wherein all of the variables are as originally defined in Class C3. In an aspect of this sub-class, all of the variables are as defined in Sub-class C3-S1.

A fifth sub-class of the third class (Sub-class C3-S5) includes compounds of Formula III-A and pharmaceutically acceptable salts thereof, wherein all of the variables are as originally defined in Class C3. In an aspect of this sub-class, all of the variables are as defined in Sub-class C3-S1.

A sixth sub-class of the third class (Sub-class C3-S6) includes compounds of Formula IV and pharmaceutically acceptable salts thereof, wherein all of the variables are as originally defined in Class C3. In an aspect of this sub-class, all of the variables are as defined in Sub-class C3-S1.

A fourth class of compounds of the present invention (Class C4) includes compounds of Formula II and pharmaceutically acceptable salts thereof, wherein:
L¹ is CH₂;
L² is CH₂ or CH₂CH₂;
X¹ and X² are each independently selected from the group consisting of H, Cl, Br, F, CN, CH₃, CF₃, OH, OCH₃, OCF₃, NH₂, N(H)CH₃, N(CH₃)₂, C(O)NH₂, C(O)N(H)CH₃, C(O)N(CH₃)₂, CH(O), C(O)CH₃, CO₂H, CO₂CH₃, SO₂H and SO₂CH₃; and provided that
   (i) at least one of X¹ and X² is other than H;
   (ii) X¹ is in the para position on the phenyl ring; and
   (iii) X² is in the meta position on the phenyl ring;
X³ is H;
Y is CH₂ or O;
Z is C(O)N(CH₃)₂, C(O)C(O)NH(CH₃), C(O)C(O)N(CH₃)₂,

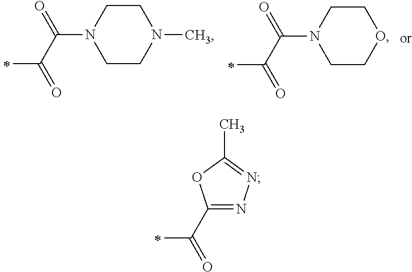

R¹ is H, CH₃, CH₂CH₃, or CH₂CH₂CH₃;
R² is H, CH₃, CH₂CH₃, OCH₃ or OH; and
R³ is H or CH₃.

A first sub-class of the fourth class (Sub-class C4-S1) includes compounds of Formula II and pharmaceutically acceptable salts thereof, wherein:
Z is C(O)N(CH₃)₂, C(O)C(O)N(CH₃)₂,

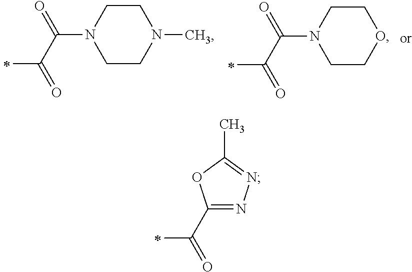

R¹ is H, CH₃, or CH₂CH₃;
R² is H; and all other variables are as originally defined in Class C4.

A second sub-class of the fourth class (Sub-class C4-S2) includes compounds of Formula II and pharmaceutically acceptable salts thereof, wherein X¹ is F; X² is H or CH₃; and all of the other variables are as originally defined in Class C4. In an aspect of this sub-class, all of the variables are as defined in Sub-class C4-S1.

A fifth class of compounds of the present invention (Class C5) includes compounds of Formula V-A:

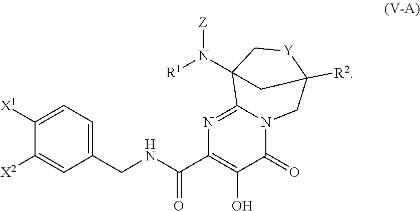

and pharmaceutically acceptable salts thereof, wherein all of the variables in Compound V-A are as originally defined.

A first sub-class of the fifth class (Sub-class C5-S1) includes compounds of Formula V-A and pharmaceutically acceptable salts thereof, wherein all of the variables in Compound V-A are as defined in Class C1.

A second sub-class of the fifth class (Sub-class C5-S2) includes compounds of Formula V-A and pharmaceutically acceptable salts thereof, wherein all of the variables in Compound V-A are as defined in Class C2. In an aspect of this sub-class, all of the variables are as defined in Sub-class C2-S1.

A third sub-class of the fifth class (Sub-class C5-S3) includes compounds of Formula V-A and pharmaceutically acceptable salts thereof, wherein all of the variables in Compound V-A are as defined in Class C3. In an aspect of this sub-class, all of the variables are as defined in Sub-class C3-S1.

A fourth sub-class of the fifth class (Sub-class C5-S4) includes compounds of Formula V-A and pharmaceutically acceptable salts thereof, wherein X¹ is F; X² is H or CH₃; and all of the other variables in Compound V-A are as defined in Class C1. In a first aspect of this sub-class, all of the other variables in Compound V-A are as defined in Class C2. In a feature of the first aspect, all of the other variables in Compound V-A are as defined in Sub-class C2-S1. In a second aspect of this sub-class, all of the other variables in Compound V-A are as defined in Class C3. In a feature of the second aspect, all of the other variables in Compound V-A are as defined in Sub-class C3-S1. In a third aspect of this sub-class, X¹ is F and X² is H. In a fourth aspect of this sub-class, X¹ is F and X² is CH₃.

A sixth class of compounds of the present invention (Class C6) includes compounds of Formula V-B:

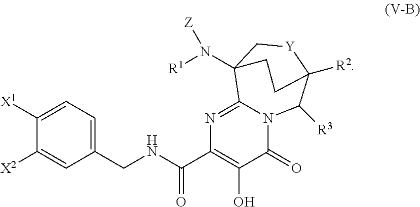

and pharmaceutically acceptable salts thereof, wherein all of the variables in Compound V-B are as originally defined.

A first sub-class of the sixth class (Sub-class C6-S1) includes compounds of Formula V-B and pharmaceutically acceptable salts thereof, wherein all of the variables in Compound V-8 are as defined in Class C1.

A second sub-class of the sixth class (Sub-class C6-S2) includes compounds of Formula V-B and pharmaceutically acceptable salts thereof, wherein all of the variables in Compound V-B are as defined in Class C2. In an aspect of this sub-class, all of the variables are as defined in Sub-class C2-S1.

A third sub-class of the sixth class (Sub-class C6-S3) includes compounds of Formula V-B and pharmaceutically acceptable salts thereof, wherein all of the variables in Compound V-B are as defined in Class C3. In an aspect of this sub-class, all of the variables are as defined in Sub-class C3-S1.

A fourth sub-class of the sixth class (Sub-class C6-S4) includes compounds of Formula V-B and pharmaceutically acceptable salts thereof, wherein X¹ is F; X² is H or CH₃; and all of the other variables in Compound V-B are as defined in Class C1. In a first aspect of this sub-class, all of the other variables in Compound V-B are as defined in Class C2. In a feature of the first aspect, all of the other variables in Compound V-B are as defined in Sub-class C2-S1. In a second aspect of this sub-class, all of the other variables in Compound V-B are as defined in Class C3. In a feature of the second aspect, all of the other variables in Compound V-B are as defined in Sub-class C3-S1. In a third aspect of this sub-class, X¹ is F and X² is H. In a fourth aspect of this sub-class, X¹ is F and X² is CH₃.

A seventh class of compounds of the present invention (Class C7) includes compounds of Formula III and pharmaceutically acceptable salts thereof, wherein:
L¹ is CH₂;
L² is CH₂ or CH₂CH₂;
X¹ and X² are each independently selected from the group consisting of H, Cl, Br, F, CN, CH₃, CF₃, OH, OCH₃, OCF₃, NH₂, N(H)CH₃, N(CH₃)₂, C(O)NH₂, C(O)N(H)CH₃, C(O)N(CH₃)₂, CH(O), C(O)CH₃, CO₂H, CO₂CH₃, SO₂H and SO₂CH₃; and provided that: (i) at least one of X¹ and X² is other than H; (ii) X¹ is in the para position on the phenyl ring; and (iii) X² is in the meta position on the phenyl ring;
X³ is H;
Y is CH₂ or O;
Z is C(O)N(CH₃)₂, C(O)C(O)NH(CH₃), C(O)C(O)N(CH₃)₂,

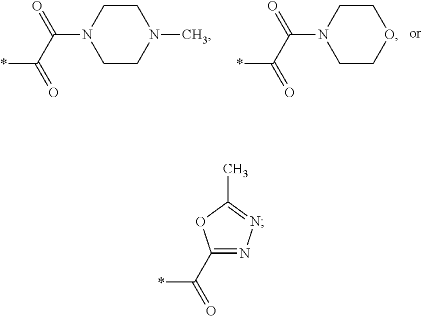

R¹ is H, CH₃, CH₂CH₃, or CH₂CH₂CH₃; and
R² is H, CH₃, CH₂CH₃, OCH₃ or OH.

A first sub-class of the seventh class (Sub-class C7-S1) includes compounds of Formula III and pharmaceutically acceptable salts thereof, wherein X¹ is F; X² is H or CH₃; and all of the other variables are as defined in Class C7.

A second sub-class of the seventh class (Sub-class C7-S2) includes compounds of Formula III and pharmaceutically acceptable salts thereof, wherein n is 1; and all other variables are as defined in Class C7. In an aspect of this sub-class, X¹ is F; X² is H or CH₃.

A third sub-class of the seventh class (Sub-class C7-S3) includes compounds of Formula III and pharmaceutically acceptable salts thereof, wherein n is zero; and all other variables are as defined in Class C7. In an aspect of this sub-class, X¹ is F; X² is H or CH₃.

An eighth class of compounds of the present invention (Class C8) includes compounds of Formula VI-A:

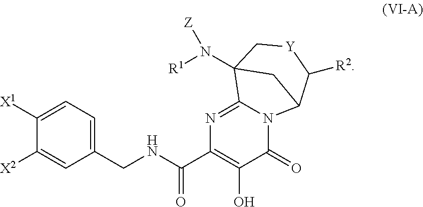

and pharmaceutically acceptable salts thereof, wherein all of the variables in Compound VI-A are as originally defined.

A first sub-class of the eighth class (Sub-class C8-S1) includes compounds of Formula VI-A and pharmaceutically acceptable salts thereof, wherein all of the variables in Compound VI-A are as defined in Class C1.

A second sub-class of the eighth class (Sub-class C8-S2) includes compounds of Formula VI-A and pharmaceutically acceptable salts thereof, wherein all of the variables in Compound VI-A are as defined in Class C2. In an aspect of this sub-class, all of the variables are as defined in Sub-class C2-S1.

A third sub-class of the eighth class (Sub-class C8-S3) includes compounds of Formula VI-A and pharmaceutically acceptable salts thereof, wherein all of the variables in Compound VI-A are as defined in Class C3. In an aspect of this sub-class, all of the variables are as defined in Sub-class C3-S1.

A fourth sub-class of the eighth class (Sub-class C8-S4) includes compounds of Formula VI-A and pharmaceutically acceptable salts thereof, wherein X¹ is F; X² is H or CH₃; and all of the other variables in Compound VI-A are as defined in Class C1. In a first aspect of this sub-class, all of the other variables in Compound VI-A are as defined in Class C2. In a feature of the first aspect, all of the other variables in Compound VI-A are as defined in Sub-class C2-S1. In a second aspect of this sub-class, all of the other variables in Compound VI-A are as defined in Class C3. In a feature of the second aspect, all of the other variables in Compound VI-A are as defined in Sub-class C3-S1. In a third aspect of this sub-class, X¹ is F and X² is H. In a fourth aspect of this sub-class, X¹ is F and X² is CH₃.

A ninth class of compounds of the present invention (Class C9) includes compounds of Formula VI-B:

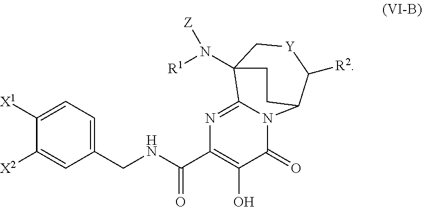

and pharmaceutically acceptable salts thereof, wherein all of the variables in Compound VI-B are as originally defined.

A first sub-class of the ninth class (Sub-class C9-S1) includes compounds of Formula VI-B and pharmaceutically acceptable salts thereof, wherein all of the variables in Compound VI-B are as defined in Class C1.

A second sub-class of the ninth class (Sub-class C9-S2) includes compounds of Formula VI-B and pharmaceutically acceptable salts thereof, wherein all of the variables in Compound VI-B are as defined in Class C2. In an aspect of this sub-class, all of the variables are as defined in Sub-class C2-S1.

A third sub-class of the ninth class (Sub-class C9-S3) includes compounds of Formula VI-B and pharmaceutically acceptable salts thereof, wherein all of the variables in Compound VI-B are as defined in Class C3. In an aspect of this sub-class, all of the variables are as defined in Sub-class C3-S1.

A fourth sub-class of the ninth class (Sub-class C9-S4) includes compounds of Formula VI-B and pharmaceutically acceptable salts thereof, wherein X¹ is F; X² is H or CH₃; and all of the other variables in Compound VI-B are as defined in Class C1. In a first aspect of this sub-class, all of the other variables in Compound VI-B are as defined in Class C2. In a feature of the first aspect, all of the other variables in Compound VI-B are as defined in Sub-class C2-S1. In a second aspect of this sub-class, all of the other variables in Compound VI-B are as defined in Class C3. In a feature of the second aspect, all of the other variables in Compound VI-B are as defined in Sub-class C3-S1. In a third aspect of this sub-class, X¹ is F and X² is H. In a fourth aspect of this sub-class, X¹ is F and X² is CH₃.

A tenth class of compounds of the present invention (Class C10) includes compounds of Formula VI-C:

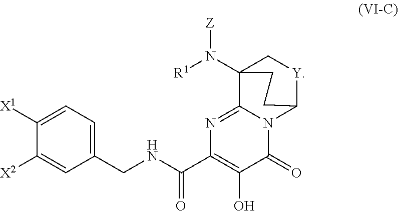

and pharmaceutically acceptable salts thereof, wherein all of the variables in Compound VI-C are as originally defined.

A first sub-class of the tenth class (Sub-class C10-S1) includes compounds of Formula VI-C and pharmaceutically acceptable salts thereof, wherein all of the variables in Compound VI-C are as defined in Class C1.

A second sub-class of the tenth class (Sub-class C10-S2) includes compounds of Formula VI-C and pharmaceutically acceptable salts thereof, wherein all of the variables in Compound VI-C are as defined in Class C2. In an aspect of this sub-class, all of the variables are as defined in Sub-class C2-S1.

A third sub-class of the tenth class (Sub-class C10-S3) includes compounds of Formula VI-C and pharmaceutically acceptable salts thereof, wherein all of the variables in Compound VI-C are as defined in Class C3. In an aspect of this sub-class, all of the variables are as defined in Sub-class C3-S1.

A fourth sub-class of the tenth class (Sub-class C10-S4) includes compounds of Formula VI-C and pharmaceutically acceptable salts thereof, wherein X¹ is F; X² is H or CH₃; and all of the other variables in Compound VI-C are as defined in Class C1. In a first aspect of this sub-class, all of the other variables in Compound VI-C are as defined in Class C2. In a feature of the first aspect, all of the other variables in Compound VI-C are as defined in Sub-class C2-S1. In a second aspect of this sub-class, all of the other variables in Compound VI-C are as defined in Class C3. In a feature of the second aspect, all of the other variables in Compound VI-C are as defined in Sub-class C3-S1. In a third aspect of this sub-class, X¹ is F and X² is H. In a fourth aspect of this sub-class, X¹ is F and X² is CH₃.

Another embodiment of the present invention is a compound of Formula I, or a pharmaceutically acceptable salt thereof, wherein the compound is selected from the group consisting of the title compounds set forth in Examples 1 to 30.

Another embodiment of the present invention is a compound of Formula I, or a pharmaceutically acceptable salt thereof, wherein the compound is selected from the group consisting of the title compounds set forth in Examples 1 to 13B.

Another embodiment of the present invention is a compound of Formula I, or a pharmaceutically acceptable salt thereof, wherein the compound is selected from the group consisting of the title compounds set forth in Examples 14 to 30.

Another embodiment of the present invention is a compound of Formula I, or a pharmaceutically acceptable salt thereof, as originally defined or as defined in any of the foregoing embodiments, sub-embodiments, classes, sub-classes, aspects and features, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt %) of a product containing a compound of Formula I or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest purity level governs. A compound or salt of 100% purity is one which is free of detectable impurities as determined by a standard method of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

The present invention also includes prodrugs of the compounds of Formula I. The term “prodrug” refers to a derivative of a compound of Formula I, or a pharmaceutically acceptable salt thereof, which is converted in viva into Compound I. Prodrugs of compounds of Formula I can exhibit enhanced solubility, absorption, and/or lipophilicity compared to the compounds per se, thereby resulting in increased bioavailability and efficacy. The in viva conversion of the prodrug can be the result of an enzyme-catalyzed chemical reaction, a metabolic chemical reaction, and/or a spontaneous chemical reaction (e.g., solvolysis). When the compound contains, for example, a hydroxy group, the prodrug can be a derivative of the hydroxy group such as an ester (—OC(O)R), a carbonate ester (—OC(O)OR), a phosphate ester (—O—P(═O)(OH)₂), or an ether (—OR). Other examples include the following: When the compound of Formula I contains a carboxylic acid group, the prodrug can be an ester or an amide, and when the compound of Formula I contains a primary amino group or another suitable nitrogen that can be derivatized, the prodrug can be an amide, carbamate, urea, imine, or a Mannich base. One or more functional groups in Compound I can be derivatized to provide a prodrug thereof. Conventional procedures for the selection and preparation of suitable prodrug derivatives are described, for example, in Design of Prodrugs, edited by H. Bundgaard, Elsevier, 1985; J. J. Hale et al., J. Med. Chem. 2000, vol. 43, pp. 1234-1241; C. S. Larsen and J. Ostergaard, “Design and application of prodrugs” in: Textbook of Drug Design and Discovery, 3^{rd} edition, edited by C. S. Larsen, 2002, pp. 410-458; and Beaumont et al., Current Drug Metabolism 2003, vol. 4, pp. 461-485; the disclosures of each of which are incorporated herein by reference in their entireties.

Other embodiments of the present invention include the following:

(a) A pharmaceutical composition comprising an effective amount of a compound of Formula I as defined above, or a prodrug or pharmaceutically acceptable salt thereof, and a pharmaceutically acceptable carrier.

(b) A pharmaceutical composition which comprises the product prepared by combining (e.g., mixing) an effective amount of a compound of Formula I as defined above, or a prodrug or pharmaceutically acceptable salt thereof, and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (e), wherein the anti-HIV agent is an antiviral selected from the group consisting of HIV protease inhibitors, HIV reverse transcriptase inhibitors, HIV integrase inhibitors, HIV fusion inhibitors, and HIV entry inhibitors.

(e) A combination which is (i) a compound of Formula I as defined above, or a prodrug or pharmaceutically acceptable salt thereof, and (ii) an anti-HIV agent selected from the group consisting of HIV antiviral agents, immunomodulators, and anti-infective agents; wherein Compound I and the anti-HIV agent are each employed in an amount that renders the combination effective for inhibition of HIV integrase, for treatment or prophylaxis of infection by HIV, or for treatment, prophylaxis of, or delay in the onset or progression of AIDS.

(f) The combination of (e), wherein the anti-HIV agent is an antiviral selected from the group consisting of HIV protease inhibitors, HIV reverse transcriptase inhibitors (nucleoside or non-nucleoside), HIV integrase inhibitors, HIV fusion inhibitors, and HIV entry inhibitors.

(g) A method for the inhibition of HIV integrase in a subject in need thereof which comprises administering to the subject an effective amount of a compound of Formula I or a prodrug or pharmaceutically acceptable salt thereof.

(h) A method for the prophylaxis or treatment of infection by HIV (e.g., HIV-1) in a subject in need thereof which comprises administering to the subject an effective amount of a compound of Formula I or a prodrug or pharmaceutically acceptable salt thereof.

(i) The method of (h), wherein the compound of Formula I is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, and HIV entry inhibitors.

(j) A method for the prophylaxis, treatment or delay in the onset or progression of AIDS in a subject in need thereof which comprises administering to the subject an effective amount of a compound of Formula I or a prodrug or pharmaceutically acceptable salt thereof.

(k) The method of (j), wherein the compound is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, and HIV entry inhibitors.

A method for the inhibition of HIV integrase in a subject in need thereof which comprises administering to the subject the pharmaceutical composition of (a), (b), (c) or (d) or the combination of (e) or (f).

(m) A method for the prophylaxis or treatment of infection by HIV (e.g., HIV-1) in a subject in need thereof which comprises administering to the subject the pharmaceutical composition of (a), (b), (c) or (d) or the combination of (e) or (f).

(n) A method for the prophylaxis, treatment, or delay in the onset or progression of AIDS in a subject in need thereof which comprises administering to the subject the pharmaceutical composition of (a), (b), (c) or (d) or the combination of (e) or (f).

The present invention also includes a compound of Formula I, or a prodrug or pharmaceutically acceptable salt thereof, (i) for use in, (ii) for use as a medicament for, or (iii) for use in the preparation of a medicament for: (a) therapy (e.g., of the human body), (b) medicine, (c) inhibition of HIV integrase, (d) treatment or prophylaxis of infection by HIV, or (e) treatment, prophylaxis of, or delay in the onset or progression of AIDS. In these uses, the compounds of the present invention can optionally be employed in combination with one or more anti-HIV agents selected from HIV antiviral agents, anti-infective agents, and immunomodulators.

Additional embodiments of the invention include the pharmaceutical compositions, combinations and methods set forth in (a)-(n) above and the uses (i) (a)-(e) through (iii) (a)-(e) set forth in the preceding paragraph, wherein the compound of the present invention employed therein is a compound of one of the embodiments, classes, sub-classes, aspects and features described above. In all of these embodiments etc., the compound may optionally be used in the form of a prodrug or a pharmaceutically acceptable salt.

Additional embodiments of the present invention include each of the pharmaceutical compositions, combinations, methods and uses set forth in the preceding paragraphs, wherein the compound of the present invention or a salt or prodrug thereof employed therein is substantially pure. With respect to a pharmaceutical composition comprising a compound of Formula I or its prodrug or salt and a pharmaceutically acceptable carrier and optionally one or more excipients, it is understood that the term “substantially pure” is in reference to a compound of Formula I or its prodrug or salt per se.

Still additional embodiments of the present invention include the pharmaceutical compositions, combinations and methods set forth in (a)-(n) above and the uses (i) (a)-(e) through (iii) (a)-(e) set forth above, wherein the HIV of interest is HIV-1. Thus, for example, in the pharmaceutical composition (d), the compound of Formula I is employed in an amount effective against HIV-1 and the anti-HIV agent is an HIV-1 antiviral selected from the group consisting of HIV-1 protease inhibitors, HIV-1 reverse transcriptase inhibitors, HIV-1 integrase inhibitors, HIV-1 fusion inhibitors and HIV-1 entry inhibitors.

As used herein, the term “alkyl” refers to a monovalent straight or branched chain, saturated aliphatic hydrocarbon radical having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and iso-propyl, ethyl and methyl. As another example, “C₁₋₄ alkyl” refers to n-, iso-, sec- and t-butyl, n- and isopropyl, ethyl and methyl.

The term “alkylene” refers to any divalent linear or branched chain aliphatic hydrocarbon radical having a number of carbon atoms in the specified range. Thus, for example, “—C₁₋₄ alkylene-” refers to any of the C₁ to C₄ linear or branched alkylenes. A class of alkylenes of interest with respect to the invention is —(CH₂)₁₋₄—, and sub-classes of particular interest include —(CH₂)₁₋₃—, —(CH₂)₂₋₃—, —(CH₂)₁₋₂—, and —CH₂—. Another sub-class of interest is an alkylene selected from the group consisting of —CH₂—, —CH(CH₃)—, and —C(CH₃)₂—.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms have been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” refers to carbonyl. The terms “S(O)₂” and “SO₂” each refer to sulfonyl. The term “S(O)” refers to sulfonyl.

An asterisk (“*”) as the end of an open bond in a chemical group denotes the point of attachment of the group to the rest of the compound.

The term “heteroaromatic ring” refers to a 5- or 6-membered heteroaromatic ring containing from 1 to 4 heteroatoms independently selected from N, O and S, wherein each N is optionally in the form of an oxide. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl) or 1,3,4-isomer), oxatriazolyl, thiazolyl, isothiazolyl, and thiadiazolyl.

Examples of 4- to 7-membered, saturated heterocyclic rings within the scope of this invention (see, e.g., the definition of HetA) include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Examples of 4- to 7-membered, unsaturated, non-aromatic heterocyclic rings within the scope of this invention include mono-unsaturated heterocyclic rings corresponding to the saturated heterocyclic rings listed in the preceding sentence in which a single bond is replaced with a double bond (e.g., a carbon-carbon single bond is replaced with a carbon-carbon double bond).

It is understood that the specific rings and ring systems suitable for use in the present invention are not limited to those listed in the preceding paragraphs. These rings and ring systems are merely representative.

Unless expressly stated to the contrary in a particular context, any of the various cyclic rings and ring systems described herein may be attached to the rest of the compound at any ring atom (i.e., any carbon atom or any heteroatom) provided that a stable compound results.

Unless expressly stated to the contrary, all ranges cited herein are inclusive. For example, a heteroaromatic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, I heteroatom, 2 heteroatoms, 3 heteroatoms, and 4 heteroatoms. As another example, a phenyl or naphthyl (see, e.g., the definition of AryA) described as optionally substituted with “from 1 to 5 substituents” is intended to include as aspects thereof, a phenyl or naphthyl substituted with 1 to 5 substituents, 2 to 5 substituents, 3 to 5 substituents, 4 to 5 substituents, 5 substituents, 1 to 4 substituents, 2 to 4 substituents, 3 to 4 substituents, 4 substituents, 1 to 3 substituents, 2 to 3 substituents, 3 substituents, 1 to 2 substituents, 2 substituents, and 1 substituent.

When any variable (e.g., R^{A} or R^{B}) occurs more than one time in any constituent or in Formula I or in any other formula depicting and describing compounds of the present invention, its definition on each occurrence is independent of its definition at every other occurrence. Also, combinations of substituents and/or variables are permissible only if such combinations result in stable compounds.

Unless expressly stated to the contrary, substitution by a named substituent is permitted on any atom in a ring provided such ring substitution is chemically allowed and results in a stable compound.

As would be recognized by one of ordinary skill in the art, certain of the compounds of the present invention can exist as tautomers. All tautomeric forms of these compounds, whether isolated individually or in mixtures, are within the scope of the present invention. For example, in instances where a hydroxy (—OH) substituent is permitted on a heteroaromatic ring and keto-enol tautomerism is possible, it is understood that the substituent might in fact be present, in whole or in part, in the keto form, as exemplified here for a hydroxypyridinyl substituent:

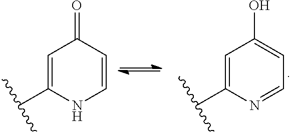

Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

A “stable compound” is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present invention are limited to stable compounds embraced by Formula I.

As a result of the selection of substituents and substituent patterns, certain compounds of the present invention can have asymmetric centers and can occur as mixtures of stereoisomers, or as individual diastereomers, or enantiomers. All isomeric forms of these compounds, whether individually or in mixtures, are within the scope of the present invention.

The atoms in a compound of Formula I may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

The methods of the present invention involve the use of compounds of the present invention in the inhibition of HIV integrase (e.g., wild type HIV-1 and/or mutant strains thereof), the prophylaxis or treatment of infection by human immunodeficiency virus (HIV) and the prophylaxis, treatment or delay in the onset or progression of consequent pathological conditions such as AIDS. Prophylaxis of AIDS, treating AIDS, delaying the onset or progression of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery. As another example, the present invention can also be employed to prevent transmission of HIV from a pregnant female infected with HIV to her unborn child or from an HIV-infected female who is nursing (i.e., breast feeding) a child to the child via administration of an effective amount of Compound I or a prodrug or pharmaceutically acceptable salt thereof.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, or benzoic acid. When compounds employed in the present invention carry an acidic moiety (e.g., —COOH or a phenolic group), suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of Formula I mean providing the compound or a prodrug of the compound to the individual in need of treatment or prophylaxis. When a compound or a prodrug thereof is provided in combination with one or more other active agents (e.g., antiviral agents useful for treating or prophylaxis of HIV infection or AIDS), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV integrase (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt form) of the compound.

In the method of the present invention (i.e., inhibiting HIV integrase, treating or prophylaxis of HIV infection or treating, prophylaxis of, or delaying the onset or progression of AIDS), the compounds of Formula I, optionally in the form of a salt or a prodrug, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the fonu of a unit dosage of a pharmaceutical composition containing an effective amount of the compound and conventional non-toxic pharmaceutically acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21st edition, Lippincott Williams & Wilkins, 2005.

The compounds of Formula I can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of a compound of Formula I with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS. Suitable HIV antivirals for use in combination with the compounds of the present invention include, for example, those listed in Table A as follows:

TABLE A

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, Atripla ® | nnRTI + nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125, Intelence ® | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| raltegravir, MK-0518, Isentress ™ | InI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, Viread ® | nRTI |
| tipranavir, Aptivus ® | PI |

EI = entry inhibitor;
FI = fusion inhibitor;
InI = integrase inhibitor;
PI = protease inhibitor;
nRTI = nucleoside reverse transcriptase inhibitor;
nnRTI = non-nucleoside reverse transcriptase inhibitor.
Some of the drugs listed in the table are used in a salt form; e.g., abacavir sulfate, delavirdine mesylate, indinavir sulfate, atazanavir sulfate, nelfinavir mesylate, saquinavir mesylate.

It is understood that the scope of combinations of the compounds of this invention with anti-HIV agents is not limited to the HIV antivirals listed in Table A, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PAR, 57^{th} edition (2003), the 58^{th} edition (2004), the 59^{th} edition (2005), and so forth. The dosage ranges for a compound of the invention in these combinations are the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention can be commercial products to be sold for these purposes.

Abbreviations employed herein include the following:
9-BBN=9-borabicyclo[3.3.1]nonane;
Bn=benzyl;
Boc=t-butyloxycarbonyl;
ACM=dichloromethane;
DIEA=diisopropylethylamine (or Hunig's base)
DMA=N,N-dimethylacetamide;
DMAP=4-dimethylaminopyridine;
DMF=N,N-dimethylformamide;
DMSO=dimethylsulfoxide;
EDC=1-ethyl-3-(3-dimethylaminopropyl) carbodiimide;
ES MS=electrospray mass spectroscopy;
Et=ethyl;
EtOAc=ethyl acetate;
EtOH=ethanol;
HMPA=hexamethylphosphoramide;
HOAT or HOAt=1-hydroxy-7-azabenzotriazole;
HPLC=high performance liquid chromatography;
HRMS=high resolution mass spectroscopy;
HR MS ESI=high resolution mass spectroscopy electrospray ionization;
LAH=lithium aluminum hydride;
LC-MS=liquid chromatography-mass spectroscopy;
LDA=lithium diisopropylamide;
LHMDS=lithium hexamethyldisilazide;
Me=methyl;
MeOH=methanol;
Ms=mesyl (or methanesulfonyl);
MTBE=methyl tert-butyl ether;
NMM=N-methylmorpholine;
NMR=nuclear magnetic resonance;
PMA=pyromellitic acid;
i-Pr=isopropyl;
RCM=ring-closing metathesis;
SFC=supercritical fluid chromatography;
TBDMS=t-butyldimethylsilyl;
TEA=triethylamine;
TFA=trifluoroacetic acid;
THF=tetrahydrofuran;
TLC=thin layer chromatography.

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

Compounds of the present invention can be prepared by coupling an esterified derivative of Q with a suitable amine. Scheme 1 exemplifies the method for Q having a 7,10-bridge, but the method can also be employed with compounds having Q groups with 6.9-bridges and 6,10 bridges. In Scheme 1 ester 1-1 containing protected amine group Pg² is reacted with a suitable, optionally substituted phenylalkylamine in a suitable organic solvent (e.g., an alkyl alcohol such as methanol or ethanol, DMSO, DMF or NMP) at a temperature in a range from about 20° C. to about 150° C. to obtain amide 1-2. Suitable methods for coupling the amine with the ester to provide an amides are described in March, Advanced Organic Chemistry, 3^{rd} edition, John Wiley & Sons, 1985, pp. 370-376. Following removal of the group Pg² in 1-2, the liberated amine is acylated to provide the desired 1-3. Suitable amine protective groups and methods for their formation and removal are described in Greene & Wuts, Protective Groups in Organic Synthesis, 2^{nd} edtion, John Wiley & Sons, 1991, pp. 309-405 and in Greene & Wuts, 3^{rd} edition, John Wiley & Sons, 1999, pp. 503-659. A suitable protective group is Boc which can be introduced by the treating the amine with di-t-butyl carbonate and subsequently removed under acidic conditions (e.g., HCl gas in dioxane/ether or a solution of trifluoroacetic acid in dichloromethane).

Acylation of the liberated amine derived from 1-2 can be carried out by coupling with various carboxylic acids (e.g., HetA-CO₂H) using procedures described in Richard Larock, Comprehensive Organic Transformations, 4^{th} edition, VCH Publishers Inc, 1989, pp 972-994, or routine variations thereof. Alternatively, the liberated amine can be reacted with one of a variety of acylating agents including acyl chlorides (e.g., HetA-C(O)Cl or HetB-C(O)Cl), carbamoyl chlorides (e.g., N(R^{A})R^{B}—C(O)Cl, sulfonyl chlorides (e.g., HetA-SO₂Cl and HetB-SO₂Cl), and sulfamoyl chlorides (e.g., N(R^{A})R^{B}—SO₂Cl) in an aprotic solvent such as a tertiary amide (e.g., DMF), an ether (e.g., THF), or a halohydrocarbon (e.g., DCM) in the presence of an organic base (e.g., a tertiary amine such as TEA, NMM or DIPEA) at a temperature of from about 0° C. to about 50° C. to afford 1-3. In yet another alternative, the liberated amine can be acylated with R^{X}—OC(O)C(O)-halide in the presence of a base (e.g., a tertiary amine such as TEA, NMM or DIPEA) in a aprotic solvent at a temperature in a range of from about 0° C. to about −20° C., wherein the resulting product is further treated with HN(R^{A})R^{B} in an alcoholic solvent (e.g., methanol or ethanol) at a temperature in the range of from about 20° C. to about 150° C. to provide oxalamides (e.g., Z═C(O)C(O)—N(R^{A})R^{B} in 1-3).

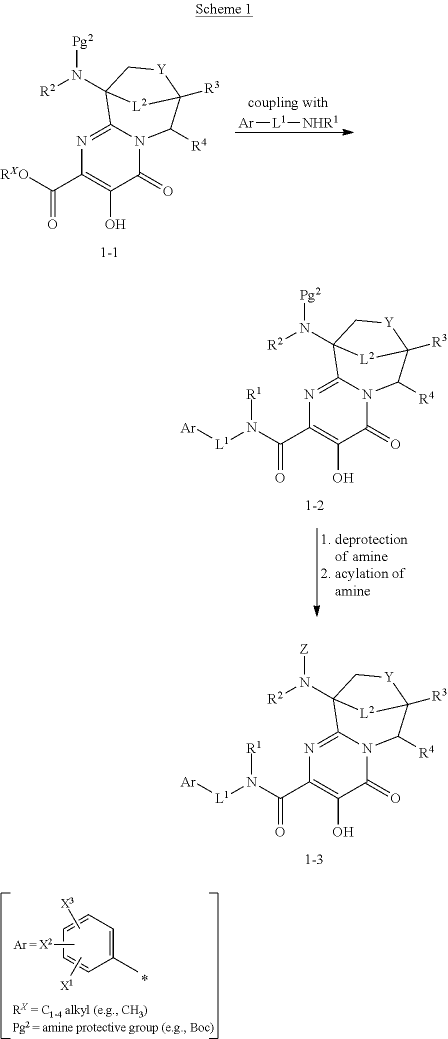

When the substitution pattern in the bridged ring system results in a chiral center in 1-1, 1-2, and 1-3, each of these compounds can exist as a mixture of enantiomers. The enantiomers can be separated at any stage in Scheme 1 by preparative HPLC or SFC methods utilizing chiral columns. Suitable procedures are described, for example, in Snyder, Kirkland, and Glajch, Practical HPLC Method Development, 2^{nd} edition, Wiley-Interscience, 1997, pp. 568-586. The separation of enantiomers can be enhanced when the phenolic hydroxy group is protected as a sulfonate ester. For example, the phenolic hydroxy group in 1-1, 1-2, or 1-3 can be sulfonylated by reacting with methanesulfonyl chloride in the presence of tertiary amine base (e.g., TEA, NMM, or DIPEA) in an aprotic solvent at a temperature in a range of from about 0° C. to about 40° C. The enantiomers can then be separated by preparative HPLC on a chiral stationary phase, after which the sulfonyl group can be removed by treatment with a base (e.g., aqueous NaOH) or a dialkylamine (e.g., Me₂NH) in alcohol (e.g., MeOH, EtOH, or i-PrOH) at 20-50° C.

Scheme 2 depicts a cyclization method suitable for formation of the bridged systems present in the compounds of the present invention. In Scheme 2, pyrimidinone intermediate 2-1 can be cyclized to 1-1 by first activating the pendant hydroxy group and then treating the resulting activated intermediate 2-2 with an inorganic base in an aprotic solvent containing water. The pendant hydroxy group can be activated by conversion to a sulfonate ester which can be obtained by treating 2-1 with a sulfonyl halide in the presence of base. The conversion to a sulfonate is exemplified in Scheme 2 as a conversion to the mesylate, which can be obtained by treating 2-1 with an excess of mesyl chloride and a tertiary amine base (e.g., TEA or DMA) in an aprotic solvent such as a halohydrocarbon (e.g., DCM), an ether (e.g., THF) or a nitrile (e.g., acetonitrile) at a temperature in a range from about 0° C. to about 40° C. to afford trimesylate intermediate 2-2. Trimesylate 2-2 can then be cyclized by treatment with base (e.g., Cs₂CO₃ or K₂CO₃) in an aprotic solvent (e.g., DMF or DMA) and optionally in the presence of 1-50 equivalents of water at temperature in a range of about 20° C. to about 160° C. to provide 1-1. Alternatively, 2-1 can be cyclized to 1-1 using Mitsunobu reaction conditions as described in J. Org. Chem. 2001, vol. 66, p. 2518-21. These conditions use a trialkylphosphonium salt such as cyanomethyptributylphosphonium iodide and a base such as TEA or DIPEA in a an aprotic solvent such as toluene or THF at a temperature in a range of from about 20° C. to about 120° C. Intermediate 1-1 can then be converted to 1-3 in the manner shown in Scheme 1.

Scheme 2 also shows an alternative cyclization route in which the alkyl carboxylate in 2-1 is first converted to amide 2-3 which can then be cyclized in the manner just described above to provide 1-2.

Cyclization methods similar to those depicted in Scheme 2 are described in WO 2005/061501.

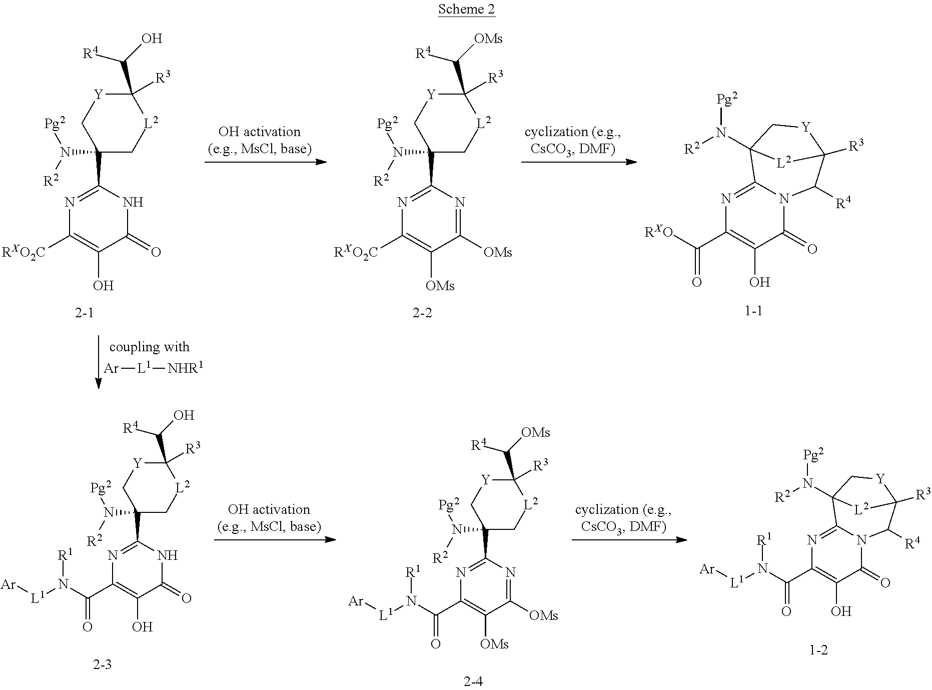

Scheme 2 depicts the cyclization for compounds having a 7,10-bridge, but the method can also be employed to provide compounds with 6.9-bridges and 6,10 bridges, as outlined in Schemes 2a and 2b.

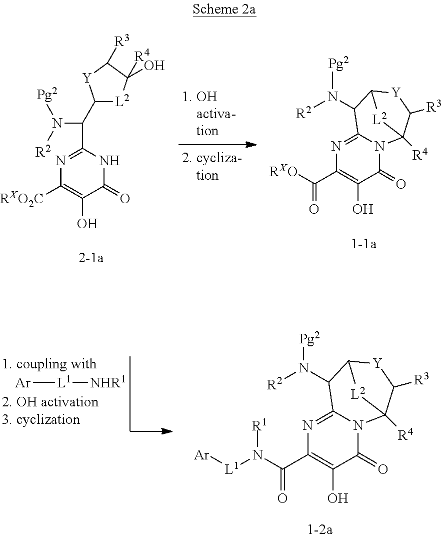

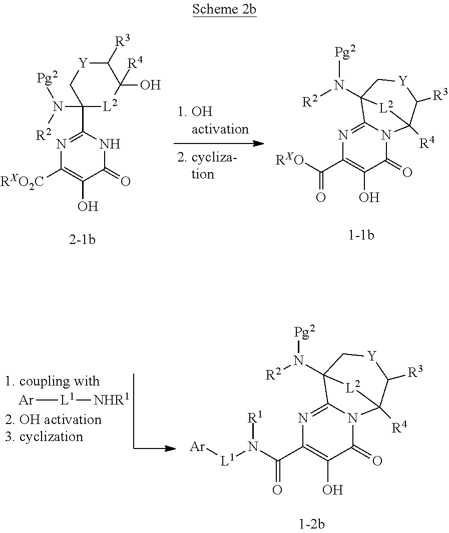

Scheme 3 shows a method for preparing the carboxylate intermediate 2-1, wherein the keto group in hydroxy protected ketone 3-1 is converted to an α-aminonitrile via the Strecker reaction, and then the amino group is protected by formation of Pg² to provide 3-2. Ketone 3-1 is treated with NaCN or KCN and the HCl salt of an amine of formula R²NH₂ in a suitable solvent such as water or alcohol (e.g., MeOH or EtOH) at a temperature in a range of from about 20° C. to about 30° C. Further description of the Strecker synthesis is in March, Advanced Organic Chemistry, 4^{th} edition, John Wiley & Sons, 1992, pp. 965-967. The hydroxy protective group Pg¹ in 3-1 can be a silyl group (e.g., TBDMS), or an arylalkyl group (e.g., benzyl). Suitable protective groups and methods for their introduction and removal are described in Greene and Wuts, Protective Groups in Organic Synthesis, 3^{rd} edition, John Wiley & Sons, 1999, pp. 503-659. The choice and introduction of amine protective group Pg² is described above with respect to Scheme 1. Intermediate 3-2 is treated with hydroxylamine in a protic solvent such as an alcohol (e.g., MeOH, EtOH, or i-PrOH) to afford hydroxyamidine 3-3, which is then reacted with a dialkyl acetylenedicarboxylate (e.g., dimethyl acetylenedicarboxylate) in a suitable solvent (e.g., MeOH, EtOH, or acetonitrile) at a temperature in a range of from about −20° C. to about 30° C. to yield butenedioate 3-4, which is then cyclized by heating (e.g., from about 90° C. to about 180° C.) under an inert atmosphere (e.g., nitrogen or argon) optionally in the presence of a base (e.g., a tertiary amine base such as TEA, DIPEA, or NMM) to afford pyrimidinone 3-5, whose OH group is then deprotected (i.e., PO is removed) to provide 2-1.

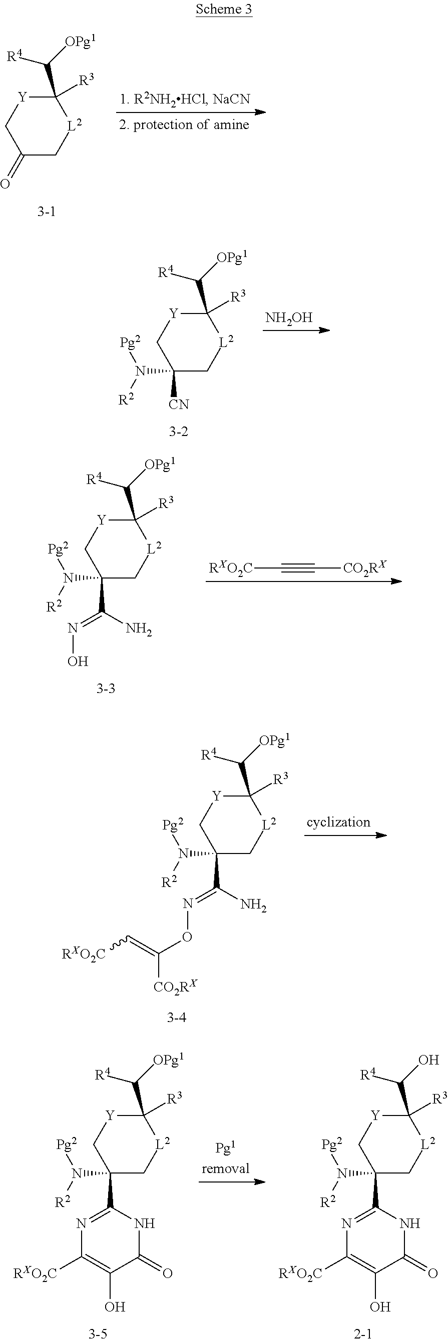

Scheme 3 depicts the preparation of the carboxylate intermediate 2-1 for compounds having a 7,10-bridge, but the method can also be employed to provide compounds with 6.9-bridges, as shown in abbreviated fashion in Scheme 3a.

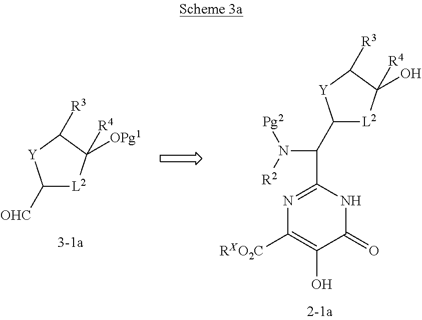

A modified version of the method of Scheme 3 can be employed to prepare compounds with 6,10-bridges, as shown in abbreviated fashion in Scheme 3b, wherein the Strecker reaction is conducted as described in Synthesis 2001, vol. 16, p. 2445-2449 to yield an α-aminonitrile product which, upon protection of its amino group, affords 3-2b′. The protected amine is then alkylated with a suitable alkylating agent such as an alkyl halide or an alkyl sulfonate ester in the presence of base (e.g., NaH, KH, LHMDS, or LDA) in an aprotic solvent (e.g., a tertiary amide such as DMF or an ether such as THF or ethyl ether) at a temperature of from about 0° C. to about 30° C. to give 3-2b″, which can then be elaborated in the manner described above in Scheme 3 to provide 2-1b.

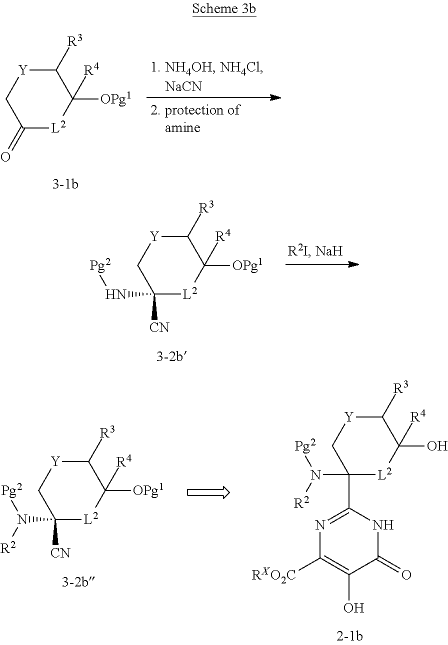

In the methods for preparing compounds of the present invention set forth in the foregoing schemes, functional groups in various moieties and substituents (in addition to those already explicitly noted in the foregoing schemes) may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2″ edition, 1991. The protecting groups may be removed at a convenient subsequent stage using methods known in the art. Alternatively the interfering group can be introduced into the molecule subsequent to the reaction step of concern.

The following examples serve only to illustrate the invention and its practice. The examples are not to be construed as limitations on the scope or spirit of the invention. In these examples, “room temperature” refers to a temperature in a range of from about 20° C. to about 25° C.

### Example 1

### N-(4-{[(4-fluorobenzyl)amino]carbonyl}-5-hydroxy-6-oxo-3,7-diazatricyclo[7.2.2.0^{2,7}]trideca-2,4-dien-1-yl)-N′,N′,N″-trimethylethanediamide

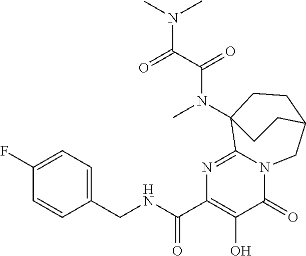

### Step 1: tert-butyl{4-trans-[(benzyloxy)methyl]-1-cyanocyclohexyl}methylcarbamate

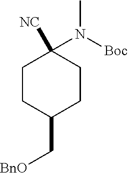

To a stirred solution of 4-benzyloxymethylcyclohexanone (synthesized in accordance with the procedure in J. Med. Chem. 1993, vol. 36, p. 654-70) (9 g, 41 mmol)] in 1:1 methanol:water (100 mL) was added methylamine hydrochloride (4.2 g, 61 mmol) and sodium cyanide (3.2 g, 61 mmol). The solution was stirred for 48 hours at room temperature. The solution was made basic (pH=9) with saturated sodium carbonate solution (50 mL). The product was extracted into ethyl acetate (3×200 mL). The ethyl acetate layers were combined, washed with brine (100 mL), and dried over anhydrous magnesium sulfate. The solvent was removed under reduced pressure. The residue was dissolved in dichloromethane (300 mL) and to the stirred solution was added di-tert-butyl dicarbonate (10 g, 47 mmol). The solution was heated to 60° C. in a closed vessel for 36 hours, cooled to room temperature and then acidified with aqueous hydrochloric acid (50 mL of a 1M solution). The organic layer was separated, washed with water (50 mL) and brine solution (50 mL), dried over magnesium sulfate, filtered, and the solvent was removed under reduced pressure. Purification of the residue by flash chromatography on a silica gel column (750 g) using a gradient elution of 5-50% ethyl acetate in hexane gave the desired product (Rf=0.5, 40% EtOAc/hexane). ¹H NMR (399 MHz, CDCl₃): δ 7.40-7.24 (m, 5H); 4.52-4.47 (m, 2H); 4.12 (q, J=7.1 Hz, 2H); 2.61 (s, 3H); 2.31-2.18 (m, 2H); 1.92-1.78 (m, 2 H); 1.83-1.61 (m, 1H); 1.62-1.18 (m, 4H). 1.42 (s, 9H). ES MS=359.3 (M+1).

### Step 2: tert-butyl{1-trans-[(E/Z)-amino(hydroxyimino)methyl]-4-[(benzyloxy)methyl]cyclohexyl}methylcarbamate

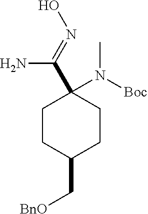

To a solution of tert-butyl {4-trans-[(benzyloxy)methyl]-1-cyanocyclohexyl}methylcarbamate (11 g, 30.7 mmol) in methanol (80 mL) was added a 50% aqueous solution of hydroxylamine (20.2 mL, 35 mmol), and the mixture was stirred at 60° C. for 18 hours. The solution was concentrated under reduced pressure. The residue was dissolved in toluene and concentrated under reduced pressure (2×50 mL) to remove traces of hydroxylamine and water. The crude product was used without purification in the next step: ES MS=392.2 (M+1).

### Step 3: Diethyl (2E/Z)-2-{[(1E/Z)-amino{4-[(benzyloxy)methyl]-1-[trans-(tert-butoxycarbonyl)(methyl)amino]cyclohexyl}methylene]amino]oxy}but-2-enedioate

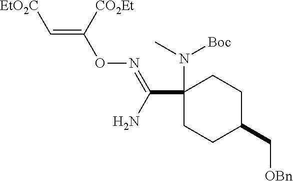

To a stirred solution of tert-butyl {1-[(E/Z)-amino(hydroxyimino)methyl]-4-trans-[(benzyloxy)methyl]cyclohexyl}methylcarbamate (10.0 g, 25.7 mmol) in methanol (100 mL) under nitrogen at 0° C. was added dimethyl acetylenedicarboxylate (3.5 mL, 28.6 mmol). The reaction was stirred at 0° C. for 2 hours and then allowed to warm to room temperature with stirring for 18 hours. The solvent was removed under reduced pressure. The residue was dissolved in toluene (50 mL) and concentrated under reduced pressure to remove traces of methanol. The crude product was used without purification in the next step: ES MS=534.2 (M+1).

### Step 4: Methyl 2-[trans-1-[(tert-butoxycarbonyl)(methyl)amino]-4-(benzyloxymethyl)cyclohexyl]-5-hydroxy-6-oxo-1,6-dihydropyrimidine-4-carboxylate

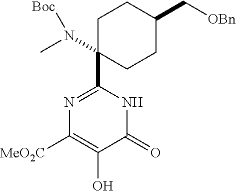

A stirred solution of diethyl (2E/Z)-2-{[(1E/Z)-amino {(4-[(benzyloxy)methyl]-1-[trans-(tert-butoxycarbonyl)(methyl)amino]cyclohexyl}methylene]amino]oxy}but-2-enedioate (10 g, 18.7 mol) in o-xylene (200 mL) under nitrogen was heated at 120° C. for 24 hours. The solution was cooled and the solvent was removed under reduced pressure. The crude product was purified by flash chromatography on a silica gel column (300 g) with a gradient elution of 0-10% methanol in dichloromethane. The product eluted at 6% methanol in dichloromethane: ES MS=502.2 (M+1).

### Step 5: Methyl 2-[trans-1-[(tert-butoxycarbonyl)(methyl)amino]-4-(hydroxymethyl)cyclohexyl]-5-hydroxy-6-oxo-1,6-dihydropyrimidine-4-carboxylate

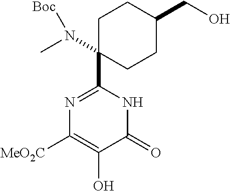

Under nitrogen atmosphere, methyl 2-[4-trans-[(benzyloxy)methyl]-1-(dimethylamino)cyclohexyl]-5-hydroxy-6-oxo-1,6-dihydropyrimidine-4-carboxylate (6.0 g, 12 mmol), ethanol (500 mL), and acetic acid (5 mL, 87 mmol) were combined. 10% Pd/C (1.0 g) was added and the mixture was shaken on a Parr apparatus under an atmosphere of hydrogen gas at 50 psi for 48 hours. The mixture was filtered through celite to remove catalyst and the filtrate solvents were removed under reduced pressure. The residue was dissolved in toluene (100 mL) and concentrated under reduced pressure to remove traces of ethanol and water. The crude product was used without purification in the next step: ES MS=412.3 (M+1).

### Step 6: Methyl 1-[(tert-butoxycarbonyl)(methyl)amino]-5-[(methylsulfonyl)oxy-6-oxo-3,7-diazatricyclo[7.2.2.0^{2,7}]trideca-2,4-diene-4-carboxylate

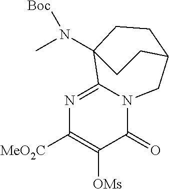

Methyl 2-[trans-1-[(tert-butoxycarbonyl)(methyl)amino]-4-(hydroxymethyl)cyclohexyl]-5-hydroxy-6-oxo-1,6-dihydropyrimidine-4-carboxylate (690 mg, 1.67 mmol) was dissolved in dry dichloromethane (15 mL) under nitrogen and cooled in an ice bath. To the stirred solution was added triethylamine (1.2 mL, 8.8 mmol) followed by methanesulfonyl chloride (0.52 mL, 6.7 mmol). The mixture was stirred for 1 hour and then diluted with water (20 mL). The organic layer was separated, washed with brine solution (20 mL), dried over anhydrous sodium sulfate, filtered, and concentrated. The crude trismesylate was used without further purification. ES MS: m/z=646.1 (M+1). Cesium carbonate (1.0 g, 3.41 mmol) was added to a stirred solution of the trismesylate (1.0 g, 1.7 mmol) in DMF (20 mL). The reaction mixture was placed in an oil bath preheated to 120° C. and stirred for 30 minutes. The solution was cooled, diluted with ethyl acetate, and filtered. The filtrate was concentrated under reduced pressure. The residue was purified by flash chromatography on a silica gel column (40 g) with a gradient elution of 30-100% ethyl acetate in hexane. The product eluted at 50% ethyl acetate in hexane. ES MS: m/z=472.2 (M+1).

### Step 7: tert-butyl (4-{[(4-fluorobenzyl)amino]carbonyl})-5-hydroxy-6-oxo-3,7-diazatricyclo[7.2.2.0^{2,7}]trideca-2,4-dien-1-yl)methylcarbamate

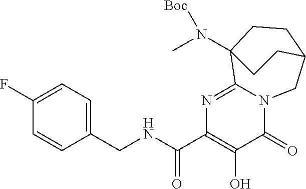

To a solution of methyl 1-[(tert-butoxycarbonyl)(methyl)amino]-5-[(methylsulfonyl)oxy-6-oxo-3,7-diazatricyclo[7.2.2.0^{2,7}]trideca-2,4-diene-4-carboxylate (500 mg, 1.27 mmol) in ethanol (10 mL) was added 4-fluorobenzylamine (0.5 mL, 3.8 mmol). The stirred solution was heated to 80° C. for 18 hours. The solution was cooled and the ethanol was removed under reduced pressure. The crude product was dissolved in ethyl acetate (50 mL) and washed with aqueous hydrochloric acid (10 mL of a 1.0 M solution). The organic layer was separated, washed successively with water and brine, dried over anhydrous magnesium sulfate, and the solvent was removed under reduced pressure. The crude product was used without further purification. ES MS: m/z=487.2 (M+1).

### Step 8: N-(4-fluorobenzyl)-5-hydroxy-1-(methylamino)-6-oxo-3,7-diazatricyclo[7.2.2.0^{2,7}]trideca-2,4-diene-4-carboxamide hydrochloride

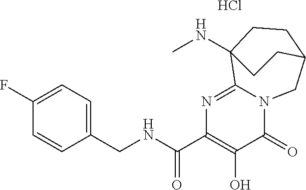

tert-Butyl (4-{[(4-fluorobenzyl)amino]carbonyl}-5-hydroxy-6-oxo-3,7-diazatricyclo[7.2.2.0^{2,7}]trideca-2,4-dien-1-yl)methylcarbamate (500 mg, 1.21 mmol) was dissolved in HCl-dioxane (10 mL of a 4 M solution) and stirred for 3 hours. The solution was concentrated under reduced pressure. The residue was suspended in toluene (20 mL) and concentrated under reduced pressure to remove traces of water. The crude product was dried under high vacuum and used without purification in the next step: ¹H NMR (599 MHz, DMSO): δ 9.96 (br.s, 1H); 9.54 (br.s, 1H); 7.42-7.34 (m, 3H); 7.13 (m, 1H); 4.50-4.43 (m, 2H); 4.20-4.14 (m, 1H); 4.02-3.97 (m, 1H); 3.94 (s, 3H); 2.43 (m, 1H); 2.18-1.96 (m, 4H); 1.85-1.75 (m, 2H); 1.68 (m, 2H). ES MS: m/z=387.2 (M+1).

### Step 9: N-(4-{[(4-fluorobenzyl)amino]carbonyl}-5-hydroxy-6-oxo-3,7-diazatricyclo[7.2.2.0^{2,7}]trideca-2,4-dien-1-yl)-1N,N′,N″-trimethylethanediamide

To a stirred solution of N-(4-fluorobenzyl)-5-hydroxy-1-(methylamino)-6-oxo-3,7-diazatricyclo[7.2.2.0^{2,7}]trideca-2,4-diene-4-carboxamide hydrochloride (134 mg, 0.35 mmol) in dry DCM (5 mL) under nitrogen was added triethylamine (194 μL, 1.4 mmol) followed by ethyl chlorooxalate (100 μL, 0.7 mmol). The reaction was stirred at room temperature for 2 hours and concentrated under reduced pressure. The residue was dissolved in methanol containing dimethylamine (5 mL of a 2 M solution) and the mixture was heated at 60° C. for 18 hours. The solution was concentrated under reduced pressure and the crude product was purified by reverse phase HPLC (Xterra C18 column) using a water:acetonitrile containing 0.1% TFA mobile phase gradient (20-70% acetonitrile over 30 minutes, 50 mL/minute). Concentration of product containing fractions gave the desired product as an amorphous white solid: ¹H NMR (599 MHz, CD₂Cl₂): δ 11.98 (br. s, 1H); 8.61 (br.s, 1H); 7.36 (dd, J=8.4, 5.4 Hz, 2H); 7.05 (dd, J=8.7, 8.7 Hz, 2H); 4.72 (dd, J=15.3, 7.5 Hz, 2H); 4.70-4.62 (m, 1H); 4.49 (dd, J=14.9, 6.0 Hz, 1H); 3.79 (s, 3H); 3.61 (d, J=15.2 Hz, 1H); 3.31 (s, 3H); 2.98 (s, 3H); 2.50 (s, 3H); 2.13-2.01 (m, 3H); 2.03-1.96 (m, 2H); 1.81-1.75 (m, 2H). HR MS: ESI=−486.2712 (M+1); calculated 486.2704 (M+1).

### Example 2

### N-(4-{[(4-fluoro-3-methylbenzyl)amino]carbonyl}-5-hydroxy-6-oxo-3,7-diazatricyclo[7.2.2.0^{2,7}]trideca-2,4-dien-1-yl)-N,N′,N″-trimethylethanediamide

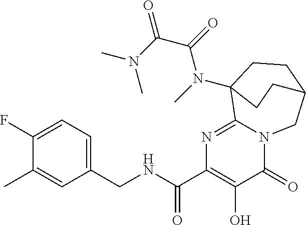

The title compound was synthesized using the procedures given in Example 1 except that 4-fluoro-3-methylbenzylamine was used in place of 4-fluorobenzylamine in Step 7. HR MS: ESI=500.2316 (M+1); calculated 500.2304 (M+1).

### Example 3

### N-(4-fluorobenzyl)-5-hydroxy-1-{methyl[morpholin-4-yl(oxo)acetyl]amino}-6-oxo-3,7-diazatricyclo[7.2.2.0^{2,7}]trideca-2,4-diene-4-carboxamide

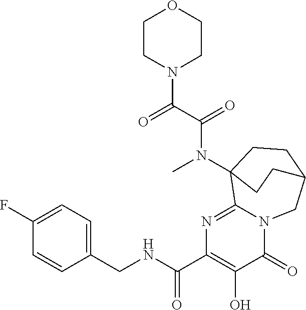

The title compound was synthesized using the procedures given in Example 1 except that morpholine was used in place of dimethylamine in Step 9.

HR MS: ESI=528.2276 (M+1); calculated 528.2253 (M+1).

### Example 4

### N-(4-fluorobenzyl)-5-hydroxy-1-{{methyl[(4-methylpiperazin-1-yl)(oxo)acetyl]amino}-6-oxo-3,7-diazatricyclo[7.2.2.0^{2,7}]trideca-2,4-diene-4-carboxamide

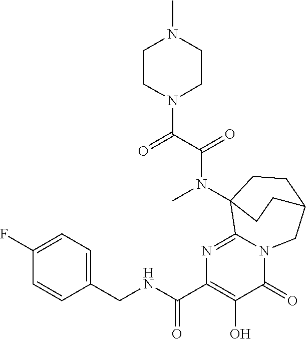

The title compound was synthesized using the procedures given in Example 1 except that 1-methylpiperazine was used in place of dimethylamine in Step 9.

HR MS: ESI=541.2608 (M+1); calculated 541.2609 (M+1).

### Example 5

### N′-{2-[(4-fluorobenzyl)carbamoyl]-3-hydroxy-4-oxo-6,7,8,9-tetrahydro-7,10-ethanopyrimido[1,2-a]azepin-10(4H)-yl}-N,N-dimethylethanediamide

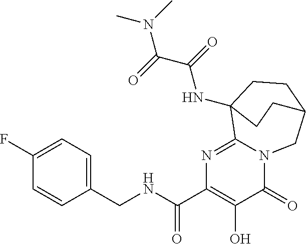

### Step 1: Ethyl 1,4-dioxaspiro[4.5]decane-8-carboxylate

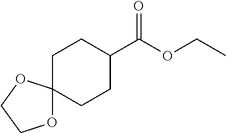

A stirred solution of ethyl 4-oxocyclohexanecarboxylate (23.5 g, 140 mmol), ethylene glycol (8.57 mL, 154 mmol), and pTsOH (0.266 g, 1.397 mmol) in toluene (250 mL) was heated to reflux under a Dean-Stark water separator for 18 hours (bath temp at 150° C.). The reaction was cooled to room temperature, washed with 25 mL of dilute NaHCO₃, and dried over anhydrous MgSO₄. Concentration under reduced pressure gave ethyl 1,4-dioxaspiro[4.5]decane-8-carboxylate as a colorless liquid.: ¹H NMR (400 MHz, CDCl₃) δ 4.07 (q, 2H), 3.92 (s, 4H), 2.35 (m, 1H), 1.95 (m, 2H), 1.8 (m, 4H), 1.55 (m, 2H), 1.24 (t, 3H).

### Step 2: 1,4-dioxaspiro[4.5]dec-8-ylmethanol

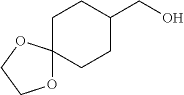

To an ice cold stirred solution of 1M LiAlH₄ in THF (180 mL, 180 mmol) was added ethyl 1,4-dioxaspiro[4.5]decane-8-carboxylate (29.8 g, 139 mmol) in THF (100 mL) slowly over 15 minutes. After warming to room temperature for 1 hour, the mixture was cooled in an ice-water bath and then quenched with water (7 mL), 6N NaOH (7 mL) and water (21 mL). The mixture was warmed to room temperature and stirred for 30 minutes. The solids were removed by filtration and the filter cake was washed with THF (3×50 mL). The filtrate was concentrated in vacuo and the residue was dissolved in toluene. The solution was concentrated in vacuo to give 1,4-dioxaspiro[4.5]dec-8-ylmethanol as a colorless liquid: ¹H NMR (400 MHz, CDCl₃) δ 3.95 (s, 4H), 3.45 (m, 2H), 1.95 (m, 1H), 1.8 (m, 4H), 1.5 (m, 2H), 1.25 (m, 2H).

### Step 3: 4-({[tert-butyl(dimethyl)silyl]oxy}methyl)cyclohexanone

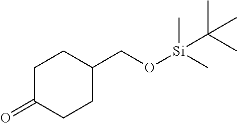

A mixture of 1,4-dioxaspiro[4.5]dec-8-ylmethanol (25 g, 145 mmol), acetone (500 mL) and 2N HCl (50 mL, 100 mmol) was stirred at 25° C. for 18 hours. The reaction mixture was concentrated in vacuo and the residue was dissolved in acetone-toluene. Concentration of the solution in vacuo gave 4-(hydroxymethyl)cyclohexanone as an oil which was used without purification. A mixture of crude 4-(hydroxymethyl)cyclohexanone (3.6 g, 28.1 mmol), imidazole (5.74 g, 84 mmol), TBDMS-Cl (6.35 g, 42.1 mmol) and DMF (8 mL) was stirred at room temperature for 18 hours. The mixture was diluted with water (200 mL) and extracted with MTBE (2×75 mL). The combined extracts were washed with water (2×50 mL) and dried over MgSO₄. Removal of solvents in vacuo gave a colorless liquid: ¹H NMR (400 MHz, CDCl₃) δ 3.5 (d, 2H), 2.35 (m, 4H), 2.05 (m, 2H), 1.9 (m, 1H), 1.4 (m, 2H), 0.85 (s, H), 0.02 (s, 6H).

### Step 4: tert-butyl[4-({[tert-butyl(dimethyl)silyl]oxy}methyl)-1-cyanocyclohexyl]carbamate

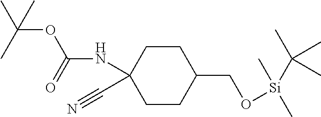

Ammonia gas was bubbled through a stirred, ice cold solution of 4-({[tert-butyl(dimethyl)silyl]oxy}methyl)cyclohexanone (6.79 g, 28 mmol) in methanol (10 mL) for 1 hour. The resulting solution was added to a stirred, ice cold mixture of KCN (5.47 g, 84 mmol) and ammonium chloride (4.94 g, 92 mmol) in ammonium hydroxide (50 mL, 360 mmol). The mixture was allowed to warm to room temperature and stirred for 18 hours in a stoppered flask. TLC (50% EtOAc/hexanes) indicated complete conversion (PMA visualization). The mixture was diluted with ethyl acetate (50 mL), filtered and concentrated in vacuo. The residue was dissolved in dioxane (10 mL) and di-tert-butyldicarbonate (12.22 g, 56.0 mmol) was added. The mixture was stirred under nitrogen for 6 hours at which time there was less than 10% conversion by TLC. After warming to 40° C. overnight, there was ˜90% conversion. More di-tert-butyldicarbonate was added (500 mg) and heating was continued for 5 hours. The mixture was concentrated in vacuo, and the residue was purified by flash chromatography on a 750 g silica gel cartridge using 0%-25% EtOAc in hexane to give the desired product: ¹H NMR (400 MHz, CDCl₃) δ 5.0 (m, 1-1H), 3.4 (d, 2H), 2.5 (m, 2H), 1.8 (m, 2H), 1.6-1.2 (m, 14H), 0.85, (s, 9H), 0.02 (s, 6H).

### Step 5: tert-Butyl[4-({[tert-butyl(dimethyl)silyl]oxy}methyl)-1-(N′-hydroxycarbamimidoyl)cyclohexyl]carbamate

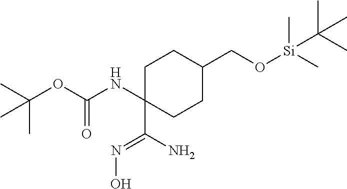

To a stirred solution of crude tert-butyl[4-({[tert-butyl(dimethyl)silyl]oxy}methyl)-1-cyanocyclohexyl]carbamate (9.5 g, 25.5 mmol) in methanol (13 mL) was added 50% aqueous hydroxylamine (2.03 mL, 33.2 mmol). The mixture was heated to 60° C. for 24 hours, then cooled and concentrated. The residue was dissolved in MeOH. The solution was concentrated in vacuo to remove traces of water and hydroxylamaine to give the desired product: ES MS=402.32 (M+1), ¹H NMR (400 MHz, CDCl₃) δ 7.04 (br s, 2H), 5.3 (m, 1H), 4.5-4.8 (m, 1H), 3.44 (m, 2H), 2.5 (m, 2H), 1.86 (d, J=14 Hz, 1H), 1.65 (m, 2H), 1.6-1.3 (m, 13H), 0.88, (s, 9H), 0.02 (s, 6H).

### Step 6: Dimethyl 2-({[amino{1-[(tert-butoxycarbonyl)amino]-4-({[tert-butyl(dimethyl)silyl]oxy}methyl)cyclohexyl}methylidene]amino}oxy)but-2-enedioate

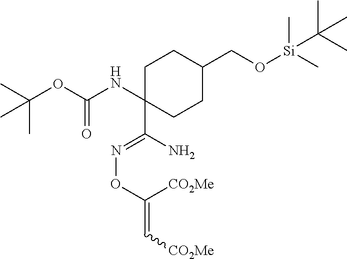

To a stirred solution of crude tert-butyl[4-({[tert-butyl(dimethyl)silyl]oxy}methyl)-1-(N′-hydroxycarbamimidoyl)cyclohexyl]carbamate (13.2 mmol) in MeOH (13 mL) cooled to −10° C. under nitrogen was added slowly dimethyl acetylenedicarboxylate (1.97 mL, 13.8 mmol) keeping internal temperature at −10° C. The resulting solution was stirred at −10 to +15° C. for 24 hours. The mixture was concentrated in vacuo to give yellow oil. Flash column chromatography eluting with 10 to 50% EtOAc/hexanes provided the desired product: ES MS=544.32 (M+1).

### Step 7: Methyl 2-{1-[(tert-butoxycarbonyl)amino]-4-({[tert-butyl(dimethyl)silyl]oxy}methyl)cyclohexyl}-5-hydroxy-6-oxo-1,6-dihydropyrimidine-4-carboxylate

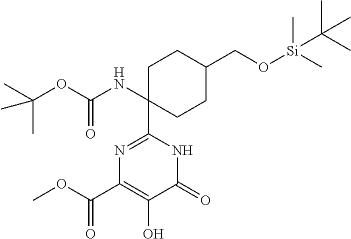

The crude dimethyl 2-({[amino{1-[(tert-butoxycarbonyl)amino]-4-({[tert-butyl(dimethyl)silyl]oxy}methyl)cyclohexyl}methylidene]amino}oxy)but-2-enedioate (3.07 g) was dissolved in o-xylene (23 mL) and heated to 115° C.±5° C. for 18 hours. The reaction turned dark soon after reaching 115° C. TLC and LCMS assay showed complete conversion. The mixture was cooled to room temperature and concentration in vacuo gave orange oil. Flash column chromatography eluting with 10 to 65% EtOAc/hexanes provided the title product as a pale yellow foam: ES MS=512.26 (M+1).

### Step 8: tert-Butyl[4-({([tert-butyl(dimethyl)silyl]oxy}methyl)-1-{4-[(4-fluorobenzyl)carbamoyl]-5-hydroxy-6-oxo-1,6-dihydropyrimidin-2-yl}cyclohexyl]carbamate

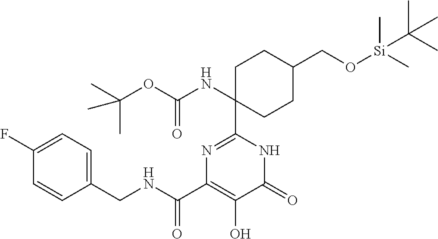

A mixture of methyl 2-{1-[(tert-butoxycarbonyl)amino]-4-({[tert-butyl(dimethyl)silyl]oxy}methyl)cyclohexyl}-5-hydroxy-6-oxo-1,6-dihydropyrimidine-4-carboxylate (1.56 g, 3.05 mmol), 4-fluorobenzylamine (0.42 g, 3.35 mmol), and TEA (0.85 mL, 6.1 mmol) in 2-propanol (60 mL) under nitrogen was heated to 78° C.±2° C. for 18 hours. The mixture was concentrated in vacuo. The residue was dissolved in isopropyl acetate (60 mL), washed successively with 10% citric acid solution (2×30 mL), 1N HCl (12 mL), water (2×12 mL), saturated aqueous NaHCO₃ 12 mL), dried over sodium sulfate, filtered, and concentrated. Drying under vacuum gave pale yellow foam. ES MS=605.31 (M+1).

### Step 9: tert-Butyl[1-{4-[(4-fluorobenzyl)carbamoyl]-5-hydroxy-6-oxo-1,6-dihydropyrimidin-2-yl}-4-(hydroxymethyl)cyclohexyl]carbamate

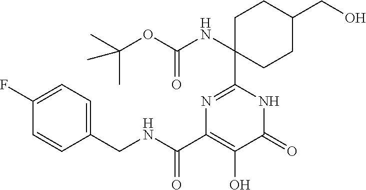

A solution of tert-butyl[4-({[tert-butyl(dimethyl)silyl]oxy}methyl)-1-{4-[(4-fluorobenzyl)carbamoyl]-5-hydroxy-6-oxo-1,6-dihydropyrimidin-2-yl}cyclohexyl]carbamate (1.65 g) in acetic acid (33 mL, 576 mmol), water (8.2 mL, 455 mmol), and THF (8.2 mL) was stirred at 40° C. for 18 hours. The solution was concentrated in vacuo. The residue was azeotropically dried with toluene(2×30 mL) on a rotary evaporator to give a solid orange foam.

ES MS=491.20.

### Step 10: tert-Butyl {2-[(4-fluorobenzyl)carbamoyl]-3-hydroxy-4-oxo-6,7,8,9-tetrahydro-7,10-ethanopyrimido[1,2-a]azepin-10(4H)-yl}carbamate

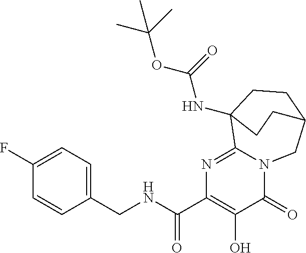

To a solution of crude tert-butyl[1-{4-[(4-fluorobenzyl)carbamoyl]-5-hydroxy-6-oxo-1,6-dihydropyrimidin-2-yl}-4-(hydroxymethyl)cyclohexyl]carbamate (1.33 g, 2.71 mmol) in DMA (11 mL) cooled in an ice-bath was added TEA (3.02 mL, 21.69 mmol), then methanesulfonyl chloride (1.479 mL, 18.98 mmol) dropwise over 15 minutes keeping the internal temperature below 10° C. The resulting slurry was stirred at ice-bath temp for 3 hours. LCMS assay showed complete conversion to a tris-mesylate intermediate: ES MS=725.1 To the ice cold solution was then added 5M aqueous NaOH (5.42 mL, 27.1 mmol). The cooling bath was removed and the stirred mixture was warmed to 80° C. for 18 hours. The mixture was cooled in an ice-bath and 3N HCl (7 mL) was added. The mixture was diluted with H2O (35 mL) and extracted with isopropyl acetate (2×30 mL). The combined extracts were washed successively with 10% citric acid solution (2×20 mL), saturated aqueous NaHCO₃ solution (3×10 mL), brine (10 mL), dried over sodium sulfate, filtered, and concentrated in vacuo to give the desired product: ES MS=473.19 (M+1).

### Step 11: 10-[(tert-Butoxycarbonyl)amino]-2-[(4-fluorobenzyl)carbamoyl]-4-oxo-4,6,7,8,9,10-hexahydro-7,10-ethanopyrimido[1,2-a]azepin-3-yl methanesulfonate

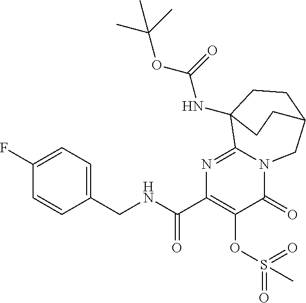

To a stirred solution of tert-butyl {2-[(4-fluorobenzyl)carbamoyl]-3-hydroxy-4-oxo-6,7,8,9-tetrahydro-7,10-ethanopyrimido[1,2-a]azepin-10(4H)-yl}carbamate (0.91 g, 1.93 mmol) and TEA (0.322 mL, 2.311 mmol) in acetonitrile (4.8 mL) cooled in an ice-bath was added in portions methanesulfonic anhydride (0.369 g, 2.118 mmol) over 3 minutes keeping the internal temperature below 15° C. The mixture was stirred at 0 to 15° C. for 30 minutes. The reaction was cooled to 0° C., quenched by addition of H₂O (4.8 mL), stirred at 0° C. for 2 hours, and extracted with isopropyl acetate (2×17 mL). The combined extracts were washed with water (8 mL), brine (4 mL), dried over sodium sulfate, filtered, and concentrated in vacuo to give a pale yellow solid foam: ES MS=551.19 (M+1).

### Step 12: 10-Amino-2-[(4-fluorobenzyl)carbamoyl]-4-oxo-4,6,7,8,9,10-hexahydro-7,10-ethanopyrimido[1,2-a]azepin-3-yl methanesulfonate hydrochloride

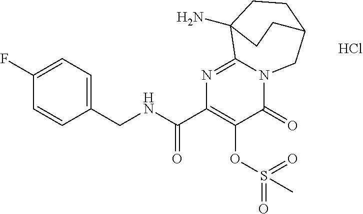

Crude 10-[(tert-butoxycarbonyl)amino]-2-[(4-fluorobenzyl)carbamoyl]-4-oxo-4,6,7,8,9,10-hexahydro-7,10-ethanopyrimido[1,2-a]azepin-3-yl methanesulfonate (0.99 g, 1.80 mmol) was dissolved in 4N HCl in dioxane (4.50 mL, 18 mmol). The mixture was stirred for 3.5 hours at room temperature and then concentrated in vacuo. Drying under vacuum gave a pale yellow solid foam: ES MS=451.15 (M+1).

### Step 13: 10-{[(Dimethylamino)(oxo)acetyl]amino}-2-[(4-fluorobenzyl)carbamoyl]-4-oxo-4,6,7,8,9,10-hexahydro-7,10-ethanopyrimido[1,2-a]azepin-3-yl methanesulfonate

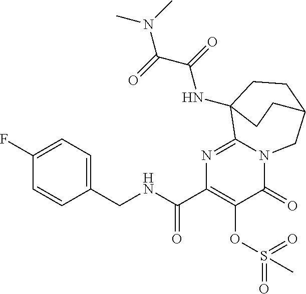

To a mixture of 10-amino-2-[(4-fluorobenzyl)carbamoyl]-4-oxo-4,6,7,8,9,10-hexahydro-7,10-ethanopyrimido[1,2-a]azepin-3-yl methanesulfonate hydrochloride (195 mg, 0.40 mmol), HOAt (82 mg, 0.60 mmol), N,N-dimethyloxamic acid (70 mg, 0.60 mmol) and triethylamine (0.223 mL, 1.60 mmol), in dichloromethane (10 mL) was added EDC (230 mg, 1.20 mmol). The mixture was stirred at room temperature under nitrogen for 18 hours, diluted with EtOAc (40 mL), washed with 10 mL 10% citric acid solution, saturated NaHCO₃ solution, water, and brine, and dried over Na₂SO₄. Filtration and concentration in vacuo gave a yellow gum: ES MS=550.18 (M+1).

### Step 14: N′-[2-{[(4-fluorobenzyl)amino]carbonyl}-3-hydroxy-4-oxo-6,7,8,9-tetrahydro-7,10-ethanopyrimido[1,2-a]azepin-10(4H)-yl]-N,N-dimethylethanediamide

To a stirred solution of crude 10-{[(dimethylamino)(oxo)acetyl]amino}-2-[(4-fluorobenzyl)carbamoyl]-4-oxo-4,6,7,8,9,10-hexahydro-7,10-ethanopyrimido[1,2-a]azepin-3-yl methanesulfonate (180 mg, 0.328 mmol) in 2-propanol (6.5 mL) was added 3M NaOH (0.109 mL, 0.328 mmol) and the mixture was stirred at room temperature for 1 hour. The reaction was concentrated and the residue was partitioned between 10% citric acid solution (4 mL) and EtOAc (40 mL). The organic layer was collected and washed sequentially with saturated aqueous NaHCO₃ solution and brine, dried over sodium sulfate, filtered, and concentrated in vacuo to give a yellow gum. The crude product was dissolved in methanol and aged at room temperature for 18 hours. The precipitate which had formed was collected by filtration and dried in vacuo to give the title compound as a white crystalline solid: HRMS (ES+): 472.1991 (M+1), ¹H NMR (400 MHz, CDCl₃) δ 12.00 (s, 1H), 8.62 (br s, 1H), 8.17 (s, 1H), 7.38 (m, 2 H), 7.02 (t, J=9 Hz, 2H), 4.56 (d, J=6 Hz, 2H), 4.17 (m, 2H), 3.29 (s, 3H), 2.92 (s, 3H), 2.51 (m, 3H), 2.09 (m, 2H), 1.97 (m, 2H), 1.72 (s, 2H).

### Example 6

### N-(4-Fluorobenzyl)-3-hydroxy-10-{[morpholin-4-yl(oxo)acetyl]amino}-4-oxo-4,6,7,8,9,10-hexahydro-7,10-ethanopyrimido[1,2-a]azepine-2-carboxamide

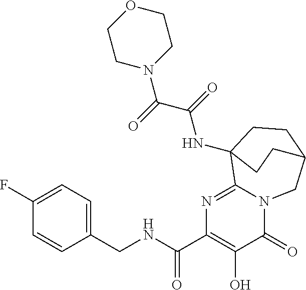

Following the procedure as described in Example 5, Steps 13 and 14 using morpholin-4-yl(oxo)acetic acid in place of N,N-dimethyloxamic acid gave crude product as a yellow gum. Purification by preparative reverse phase chromatography (gradient elution 0.1% acetic acid in water/acetonitrile) gave the title compound as an off-white crystalline solid: HRMS (ES+): 514.2107 (M+1), ¹H NMR (400 MHz, CDCl₃) δ 12.00 (br s, 1H), 8.48 (br s, 1H), 8.40 (s, 1H), 7.37 (m, 2H), 7.02 (t, J=7 Hz, 2H), 4.55 (d, J=6 Hz, 2H), 4.17 (d, J=4 Hz, 2H), 4.02 (m, 2H), 3.70 (m, 4H), 3.53 (m, 2H), 2.5-2.6 (m, 3H), 1.9-2.1 (m, 4H), 1.72 (m, 2H).

### Example 7

### N-{2-[(4-fluorobenzyl)carbamoyl]-3-hydroxy-4-oxo-6,7,8,9-tetrahydro-7,10-methanopyrimido[1,2-a]azepin-10(4H)-yl}-N,N′,N′-trimethylethanediamide

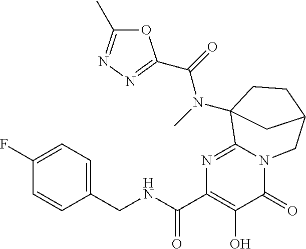

### Step 1: 3-[(E and Z)-2-phenylethenyl]cyclopentanone

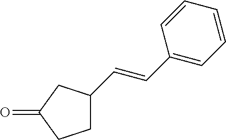

To a stirred mixture of 2-cyclopenten-1-one (25 g, 305 mmol) and bis(acetonitrile) (1,5-cyclooctadiene)rhodium(I)tetrafluoroborate (2.314 g, 6.09 mmol) in dioxane (300 mL) and water (30 mL) was added trimethoxy[(E)-2-phenylethenyl]silane (82 g, 365 mmol; prepared by the method of A. Wienand and H.-U. Reissig, Organometallics, 1990, volume 9, p. 3133-3142), after which the mixture was heated to 900° C. for 20 hours. MTBE (1000 mL) was then added to the reaction mixture and the precipitate was removed by filtration through diatomaceous earth (3×50 mL rinse of filter pad with MTBE). The filtrate was concentrated in vacuo. The residue was purified by flash chromatography on a 750 g silica gel cartridge using a mobile phase gradient of 0%-20% EtOAc/hexane. 3-[(E and Z)-2-phenylethenyl]cyclopentanone was obtained as an oil which crystallized under vacuum overnight: ¹H NMR (400 MHz, CDCl₃) δ 7.3 (m, 5H), 6.4 (m, 1H), 6.2 (m, 0.75H), 5.6 (t, 0.25H), 3.35 (m, 0.25H), 3.0 (m, 0.75H), 2.6-2.0 and 1.8 (complex m, 6H); ES MS M+1=187.19.

### Step 2: 7-[(E and Z)-2-phenylethenyl]-1,4-dioxaspiro[4.4]nonane

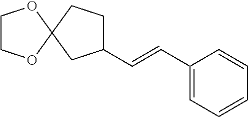

A stirred solution of 3-[(E and Z)-2-phenylethenyl]cyclopentanone (24 g, 129 mmol), ethylene glycol (7.90 mL, 142 mmol), and pTsOH (0.245 g, 1.289 mmol) in toluene (200 mL) was heated to reflux under a Dean-Stark water separator for 18 hours (bath temperature at 150° C.). The mixture was cooled to room temperature, diluted with MTBE (50 mL), washed with dilute NaHCO₃(25 mL), and dried over MgSO₄. Filtration and concentration in vacuo gave a colorless liquid.: ¹H NMR (400 MHz, CDCl₃) δ 7.4-7.15 (m, 5H), 6.4 (d, 1H), 6.2 (dd, 1H), 3.9 (m, 4H), 2.8 (m, 1H), 2.1-1.5 and 1.8 (complex m, 6H); ES MS M+1=231.17.

### Step 3: 1,4-dioxaspiro[4.4]non-7-ylmethanol

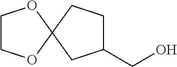

A stream of ozone (5.63 g, 117 mmol) was introduced via a gas dispersion tube into a stirred solution of 7-[(E and Z)-2-phenylethenyl]-1,4-dioxaspiro[4.4]nonane (27 g, 117 mmol) in MeOH (50 mL) and CH₂Cl₂ (50 mL) cooled in a dry-ice acetone bath to −70° C. until a blue color persisted (2 hours). The ozone stream was stopped, the mixture was stirred for 10 minutes, and then the solution was purged with nitrogen until it was colorless. NaBH₄ (8.87 g, 234 mmol) was added and after the exotherm subsided, the mixture was allowed to warm to room temperature with stirring for 18 hours. The reaction mixture tested negative (no color) to a peroxide test strip. The mixture was concentrated in vacuo and diluted with ethyl acetate (750 mL) and water (100 mL). The organic layer was separated, washed with brine (50 mL), dried over MgSO₄, and filtered. The residue after concentration in vacuo was purified on a 750 g silica gel column eluting with 0% to 75% MTBE in hexanes to give 1,4-dioxaspiro[4.4]non-7-ylmethanol: ¹H NMR (400 MHz, CDCl₃) δ 3.9 (s, 4H), 3.6 (m, 2H), 2.25 (m, 1H), 2.0 (m, 2H), 1.95 (m, 2H), 1.6 (m, 1H), 1.5 (m, 1H); ES MS M+1=159.13.

### Step 4: 3-(hydroxymethyl)cyclopentanone

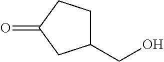

A mixture of 1,4-dioxaspiro[4.4]non-7-ylmethanol (15 g, 95 mmol), THF (125 mL) and 2N HCl (47.4 mL, 95 mmol) was stirred at 25° C. for 24 hours. The mixture was concentrated under reduced pressure, diluted with 250 mL of THF, cooled in an ice bath, and ammonia gas (16.15 g, 948 mmol) was dispersed into the solution for 10 minutes. The organic phase was collected and the aqueous phase which contained a thick white precipitate was extracted with 50% THF in ethyl acetate (3×50 mL). The combined organic phases were dried over MgSO₄, filtered, and concentrated in vacuo to give a clear oil: ¹H NMR (400 MHz, CDCl₃) 3.6 (m, 2H), 2.5-2.0 (complex m, 6H), 1.7 (m, 1H); ES MS M+1=115.00.

### Step 5: 3-({[tert-butyl(dimethyl)silyl]oxy}methyl)cyclopentanone

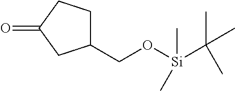

A mixture of 3-(hydroxymethyl)cyclopentanone (10 g, 88 mmol), imidazole (17.89 g, 263 mmol), TBDMS-C (19.81 g, 131 mmol) in DMF (20 mL) was stirred at room temperature in a stoppered flask for 18 hours. Another 3.2 g of imidazole and 3.5 g of TBDMS-Cl were added and the mixture was stirred at room temperature for 24 hours. The mixture was diluted with of water (200 mL) and extracted with MTBE (2×75 mL). The combined organic extracts were washed with water (2×50 mL), dried over MgSO₄, and the solvents were removed in vacuo to give a colorless liquid: ¹H NMR (400 MHz, CDCl₃) δ 3.6 (dd, 2H), 2.4-2.0 (complex m, 6H), 1.7 (m, 1H), 0.9 (s, 9H), 0.02 (s, 6H).

### Step 6: racemic cis and trans tert-butyl[3-({[tert-butyl(dimethyl)silyl]oxy}methyl)-1-cyanocyclopentyl]methylcarbamate

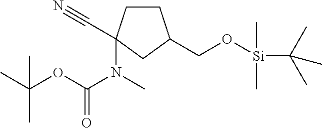

To an ice cold stirred mixture of 3-({[tert-butyl(dimethyl)silyl]oxy}methyl)cyclopentanone (20.10 g, 88 mmol) and methylamine hydrochloride (17.82 g, 264 mmol) in dioxane (40 mL) was added sodium cyanide (12.94 g, 264 mmol) and water (40.0 mL). The mixture was stirred in a stoppered flask for 24 hours. TLC (10% EtOAc/hex) indicated incomplete consumption of starting material. Another 8 g of methylamine hydrochloride and 6 g of sodium cyanide were added and the mixture was stirred for 24 hours. The mixture was extracted with isopropyl acetate (3×150 mL) and the combined extracts were dried over MgSO₄, filtered, and concentrated in vacuo. The residue was dissolved in isopropyl acetate (250 mL), di-tert-butyl dicarbonate (38.4 g, 176 mmol) was added, and the resulting mixture was stirred at room temperature for 18 hours. Another 10 g of di-tertbutyldicarbonate was added, the mixture was stirred for 24 hours and then concentrated in vacuo. The residue was purified by flash chromatography on a 750 g silica gel cartridge using a gradient elution of 0%-10% EtOAc in hexane to give two isomers as colorless oils:

Isomer A—¹H NMR (400 MHz, CDCl₃) δ 3.5 (d, 2H), 2.9 (s, 3H), 2.5 (m, 2H), 2.4 (m, 1H), 1.9 (m, 2H), 1.75 (m, 1H), 1.6 (m, 1H), 1.45 (s, 9H), 0.9 (s, 9H), 0.02 (s, 6H); ES MS M+1=369.22.

Isomer B1: ¹H NMR (400 MHz, CDCl₃) δ 3.5 (d, 2H), 2.9 (s, 3H), 2.35 (m, 2H), 2.2 (m, 2H), 1.9 (m, 2H), 2.05 (m, 1H), 1.9 (m, 1H), 1.6 (m, 1H), 1.45 (s, 9H), 0.9 (s, 9H), 0.02 (s, 6H); ES MS M+1=369.22.

### Step 7: trans-tert-Butyl[3-({[tert-butyl(dimethyl)silyl]oxy}methyl)-1-(N′-hydroxycarbamimidoyl)cyclopentyl]methylcarbamate

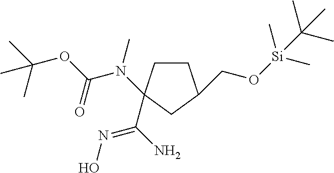

To a stirred solution of tert-butyl[3-({[tert-butyl(dimethyl)silyl]oxy}methyl)-1-cyanocyclopentyl]methylcarbamate isomer B (7.5 g, 20.35 mmol), in methanol (10 mL) was added 50% hydroxylamine (1.62 mL, 26.5 mmol). The mixture was heated to 60° C. for 18 hours, cooled, and concentrated in vacuo. Removal of excess hydroxylamine and water by concentration from methanol and drying in vacuo gave the desired product: ES MS=402.26 (M+1), ¹H NMR (400 MHz, CDCl₃) δ 7.0 (br s, 1H), 5.08 (s, 2H), 3.6-3.5 (m, 2H), 3.00 and 2.87 (2 singlets, 3H), 2.4-2.0 (m, 4H), 1.8-1.5 (m, 3H), 1.45 (s, 9H), 0.88 (s, 9H), 0.04 (s, 6H).

### Step 8: Dimethyl 2-({[amino {trans-1-[(tert-butoxycarbonyl)(methyl)amino]-3-({[tert-butyl(dimethyl)silyl]oxy}methyl)cyclopentyl}methylidene]amino}oxy)but-2-enedioate

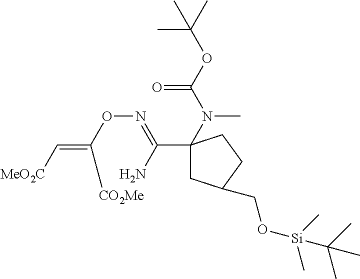

To a stirred solution of crude trans-tert-butyl[3-({[tert-butyl(dimethyl)silyl]oxy}methyl)-1-(N′-hydroxycarbamimidoyl)cyclopentyl]methylcarbamate (13.7 mmol) in MeOH (14 mL) cooled to −10° C. under nitrogen was added slowly dimethyl acetylenedicarboxylate (1.77 mL, 14.4 mmol) keeping the internal temperature at −10° C. The resulting solution was stirred at −10 to +15° C. for 18 hours. The mixture was concentrated in vacuo to give a yellow oil which was purified by passage through a pad of silica gel eluting with 25% EtOAc/hexanes to give the desired product: ES MS=544.32 (M+1).

### Step 9: Methyl 2-{trans-1-[(tert-butoxycarbonyl)(methyl)amino]-3-({[tert-butyl(dimethyl)silyl]oxy)}methyl)cyclopentyl}-5-hydroxy-6-oxo-1,6-dihydropyrimidine-4-carboxylate

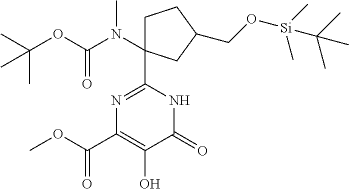

Dimethyl 2-({[amino{trans-1-[(tert-butoxycarbonyl)(methyl)amino]-3-({[tert-butyl(dimethyl)silyl]oxy}methyl)cyclopentyl}methylidene]amino}oxy)but-2-enedioate (6.95 g) was dissolved in o-xylene (51 mL), and heated at 115° C.±5° C. for 24 hours. The reaction turned dark soon after reaching 115° C. The mixture was cooled to room temperature and concentrated in vacuo. The resulting orange oil was purified by flash column chromatography on a silica gel column eluting with 10 to 65% EtOAc/hexanes to give a pale yellow foam: ES MS=512.25 (M+1).

### Step 10: tert-Butyl[trans-3-({[tert-butyl(dimethyl)silyl]oxy}methyl)-1-{4-[(4-fluorobenzyl)carbamoyl]-5-hydroxy-6-oxo-1,6-dihydropyrimidin-2-yl}cyclopentyl]methylcarbamate

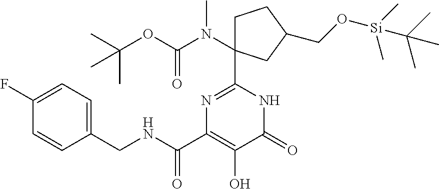

A mixture of methyl 2-{trans-1-[(tert-butoxycarbonyl)(methyl)amino]-3-({[tert-butyl(dimethyl)silyl]oxy}methyl)cyclopentyl}-5-hydroxy-6-oxo-1,6-dihydropyrimidine-4-carboxylate (3.37 g, 6.59 mmol), 4-fluorobenzylamine (0.907 g, 7.24 mmol), and TEA (1.84 mL, 13.2 mmol) in 2-propanol (132 mL) was heated under nitrogen to 78° C.±2° C. for 18 hours. The mixture was concentrated in vacuo. The residue was dissolved in of isopropyl acetate (115 mL), washed with 10% citric acid solution (2×65 mL), 1N HCl (30 mL), water (2×25 mL), saturated aqueous NaHCO₃ (25 mL), dried over anhydrous sodium sulfate, filtered, and concentrated in vacuo to give a pale yellow foam: ES MS=605.30 (M+1).

### Step 11: tert-Butyl[trans-1-{4-[(4-fluorobenzyl)carbamoyl]-5-hydroxy-6-oxo-1,6-dihydropyrimidin-2-yl}-3-(hydroxymethyl)cyclopentyl]methylcarbamate

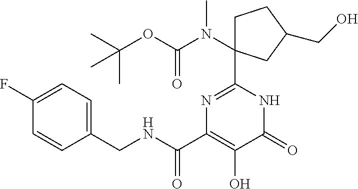

A solution of tert-butyl[trans-3-({[tert-butyl(dimethyl)silyl]oxy}methyl)-1-{4-[(4-fluorobenzyl)carbamoyl]-5-hydroxy-6-oxo-1,6-dihydropyrimidin-2-yl}cyclopentyl]methylcarbamate (3.29 g) in acetic acid (66 mL, 1153 mmol), water (16.5 mL, 916 mmol), and THF (16.5 mL) was stirred at 40° C. for 18 hours. The solution was concentrated in vacuo and he residue was azeotropically dried with toluene (2×60 mL) in vacuo to give an orange foam: ES MS=491.20.

### Step 12: tert-Butyl {2-[(4-fluorobenzyl)carbamoyl]-3-hydroxy-4-oxo-6,7,8,9-tetrahydro-7,10-methanopyrimido[1,2-a]azepin-10(4H)-yl}methylcarbamate

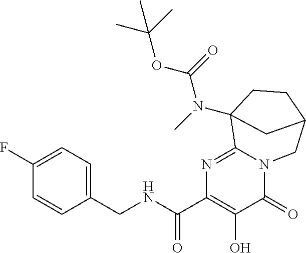

Part 1—To a stirred solution of tert-butyl[trans-1-{4-[(4-fluorobenzyl)carbamoyl]-5-hydroxy-6-oxo-1,6-dihydropyrimidin-2-yl}-3-(hydroxymethyl)cyclopentyl]methylcarbamate (2.72 g, 5.55 mmol) in DMA (22 mL) cooled in an ice-bath was added TEA (6.18 mL, 44.4 mmol) followed by methanesulfonyl chloride (3.02 mL, 38.8 mmol) added dropwise over 20 minutes and keeping the internal temperature below 10° C. The resulting slurry was stirred at ice-bath temperature for 3 hours. LCMS assay showed complete conversion to a tris-mesylate intermediate: ES MS=725.1

Part 2—5M NaOH aqueous solution (11.1 mL, 55.5 mmol) was added dropwise to the chilled slurry. The mixture was then warmed to 80° C. and stirred for 18 hours. The mixture was cooled in an ice-bath and 3N HCl (14 mL) was added. The mixture was diluted with H₂O (70 mL) and extracted with isopropyl acetate (2×60 mL). The combined extracts were washed with 10% aqueous citric acid (2×40 mL), of saturated aqueous NaHCO₃ (3×20 mL), and brine (20 mL). The solution was dried over sodium sulfate, filtered, concentrated in vacuo to give the desired product: ES MS=473.19 (M+1).

### Step 13: 10-[(tert-Butoxycarbonyl)(methyl)amino]-2-[(4-fluorobenzyl)carbamoyl]-4-oxo-4,6,7,8,9,10-hexahydro-7,10-methanopyrimido[1,2-a]azepin-3-yl methanesulfonate

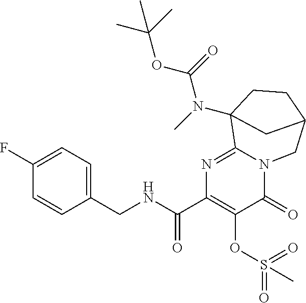

To an ice cold stirred solution of tert-butyl {2-[(4-fluorobenzyl)carbamoyl]-3-hydroxy-4-oxo-6,7,8,9-tetrahydro-7,10-methanopyrimido[1,2-a]azepin-10(4H)-yl}methylcarbamate (1.76 g, 3.72 mmol) and TEA (0.623 mL, 4.47 mmol) in acetonitrile (9.5 mL) was added methanesulfonic anhydride (0.714 g, 4.10 mmol) in several portions over 3 minutes, keeping the internal temperature below 15° C. The mixture was stirred at 0 to 15° C. for minutes. The reaction was cooled to 0° C., quenched by the addition of H₂O (9.5 mL), stirred at 0° C. for 2 hours, and extracted with of isopropyl acetate (2×35 mL). The combined organic extracts were washed with water (10 mL), brine (8 mL), dried over sodium sulfate, filtered, and concentrated in vacuo to give the desired product as a brown solid foam: ES MS=551.19 (M+1).

### Step 14: 2-[(4-Fluorobenzyl)carbamoyl]-10-(methylamino)-4-oxo-4,6,7,8,9,10-hexahydro-7,10-methanopyrimido[1,2-a]azepin-3-yl methanesulfonate hydrochloride

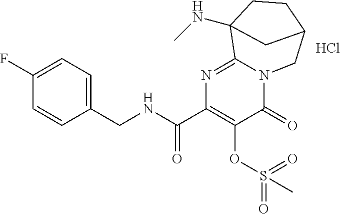

10-[(tert-Butoxycarbonyl)(methyl)amino]-2-[(4-fluorobenzyl)carbamoyl]-4-oxo-4,6,7,8,9,10-hexahydro-7,10-methanopyrimido[1,2-a]azepin-3-yl methanesulfonate (1.95 g, 3.54 mmol) was dissolved in 4N HCl in dioxane (8.85 mL, 35.4 mmol) and the mixture was stirred for 2 hours. The solution was concentrated in vacuo to give a pale yellow solid foam: ES MS=451.15 (M+1).

### Step 15: 2-[(4-Fluorobenzyl)carbamoyl]-10-{methyl[(5-methyl-1,3,4-oxadiazol-2-yl)carbonyl]amino}-4-oxo-4,6,7,8,9,10-hexahydro-7,10-methanopyrimido[1,2-a]azepin-3-yl methanesulfonate

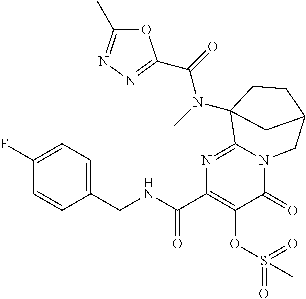

To a mixture of 2-[(4-fluorobenzyl)carbamoyl]-10-(methylamino)-4-oxo-4,6,7,8,9,10-hexahydro-7,10-methanopyrimido[1,2-a]azepin-3-yl methanesulfonate hydrochloride (195 mg, 0.40 mmol), HOAt (82 mg, 0.60 mmol), potassium 5-methyl-1,3,4-oxadiazole-2-carboxylate (100 mg, 0.60 mmol), triethylamine hydrochloride (83 mg, 0.60 mmol), and triethylamine (0.167 mL, 1.20 mmol) in dichloromethane (8 mL) was added EDC (230 mg, 1.20 mmol). The mixture was stirred under nitrogen at room temperature for 18 hours. The mixture was diluted with EtOAc (40 mL) and then washed with 10 mL each of 10% aqueous citric acid, saturated aqueous NaHCO₃, and brine. The solution was dried over Na₂SO₄ and concentrated in vacuo to give a yellow gum: ES MS=561.16 (M+1).

### Step 16: N-(4-Fluorobenzyl)-3-hydroxy-10-{methyl[(5-methyl-1,3,4-oxadiazol-2-yl)carbonyl]amino}-4-oxo-4,6,7,8,9,10-hexahydro-7,10-methanopyrimido[1,2-a]azepine-2-carboxamide

To a stirred solution of 2-[(4-fluorobenzyl)carbamoyl]-10-{methyl[(5-methyl-1,3,4-oxadiazol-2-yl)carbonyl]amino}-4-oxo-4,6,7,8,9,10-hexahydro-7,10-methanopyrimido[1,2-a]azepin-3-yl methanesulfonate (79 mg, 0.14 mmol) in 2-propanol (2.8 mL) was added 3M NaOH (0.047 mL, 0.14 mmol) and the mixture was stirred at room temperature for 2.5 hours. The reaction was concentrated in vacuo and the residue was partitioned between 2 mL of 10% aqueous citric acid solution and 20 mL of EtOAc. The organic phase was collected and washed with 4 mL each of saturated aqueous NaHCO₃ and brine, dried over sodium sulfate, filtered, and concentrated in vacuo. The residue was purified by preparative reverse phase chromatography (gradient elution 0.1% acetic acid in water/acetonitrile) to give a white crystalline solid: HRMS (ES+): 483.1786 (M+H), ¹H NMR (400 MHz, CDCl₃) δ 12.0 (br s, 1H), 8.2 (br s, 1H), 7.32 (m, 2H), 7.00 (t, J=9 Hz, 2H), 4.55 (m, 1H), 4.48 (m, 1H), 4.06 (d, J=13 Hz, 1H), 3.86 (m, 1H), 3.49 (s, 3H), 3.1-3.3 (m, 2H), 2.87 (br s, 1H), 2.58 (s, 3H), 2.0-2.5 (m, 4H).

### Example 8

### N-({2-[(4-Fluorobenzyl)carbamoyl]-3-hydroxy-4-oxo-6,7,8,9-tetrahydro-7,10-methanopyrimido[1,2-a]azepin-10(4H)-yl}-N,N′,N′-trimethylethanediamide

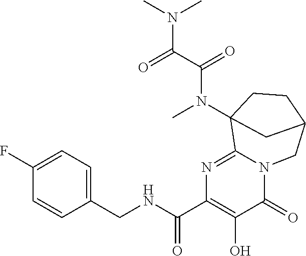

Following the procedure as described in Example 5, Steps 13 and 14 starting with 2-[(4-fluorobenzyl)carbamoyl]-10-(methylamino)-4-oxo-4,6,7,8,9,10-hexahydro-7,10-methanopyrimido[1,2-a]azepin-3-yl methanesulfonate hydrochloride (from Step 14 of Example 7) gave a yellow gum. Two crystallizations from methanol gave the title compound as a white

crystalline solid: HRMS (ES+): 472.1993 (M+1), ¹H NMR (400 MHz, CDCl₃) δ 12.2 (s, 0.5H), 12.0 (s, 0.5H), 9.8 (br s, 0.5H), 9.3 (br s, 0.5H), 7.38 (m, 2H), 6.99 (t, J=8 Hz, 2H), 4.5 (m, 2H), 4.02 (m, 1H), 3.81 (br s, 1H), 3.49 (d, J=5 Hz, 1H), 2.8-3.3 (m, 10H), 2.2-2.5 (m, 3H), 1.6-2.0 (m, 2H).

### Example 9

### N-(4-Fluorobenzyl)-3-hydroxy-10-{methyl[morpholin-4-yl(oxo)acetyl]amino}-4-oxo-4,6,7,8,9,10-hexahydro-7,10-methanopyrimido[1,2-a]azepine-2-carboxamide

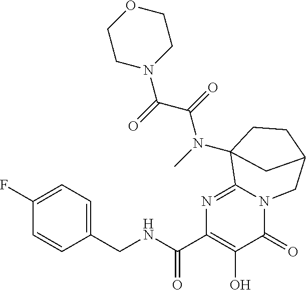

Following the procedure as described in Example 8, starting with 2-[(4-fluorobenzyl)carbamoyl]-10-(methylamino)-4-oxo-4,6,7,8,9,10-hexahydro-7,10-methanopyrimido[1,2-a]azepin-3-yl methanesulfonate hydrochloride (from Step 14 of Example 7) gave a yellow gum. Purification by preparative reverse phase chromatography (gradient elution 0.1% acetic acid in water/acetonitrile) gave the title compound as a pale orange crystalline solid: HRMS (ES+): 514.2100 (M+1). ¹H NMR (400 MHz, CDCl₃) δ 12.2 (s, 0.5H), 12.0 (s, 0.5H), 9.6 (br s, 0.5H), 9.2 (br s, 0.5H), 7.36 (m, 2H), 6.99 (t, J=7 Hz, 2H), 4.5 (m, 2H), 4.02 (m, 1H), 3.4-3.9 (m, 9H), 2.8-3.3 (m, 5H), 1.9-2.4 (m, 4H), 1.75 (m, 1H).

### Example 10A

### N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′N′-trimethylethanediamide

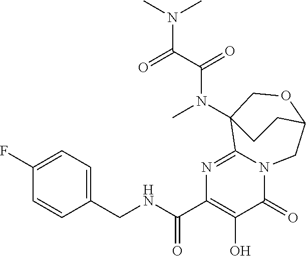

### Step 1: 2-[(benzyloxy)methyl]-3,4-dihydro-2H-pyran

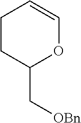

A stirred suspension of sodium hydride (5.26 g of a dispersion in 60% mineral oil, 131 mmol) in dry DMF (100 mL) under a nitrogen atmosphere was cooled in an ice bath. 2-Hydroxymethyl 3,4-dihydro-2H-pyran (15 mL, 131 mmol) was added dropwise over 30 minutes and the resulting mixture was stirred for 2 hours at 0° C. Benzyl bromide (16 mL, 133 mmol) was added and the stirred reaction mixture was allowed to warm to room temperature over 18 hours. The reaction was quenched with saturated aqueous ammonium chloride (100 mL) and the product was extracted into ether (2×200 mL). The organic layers were combined and washed successively with water and brine solution. The organic layer was dried over anhydrous magnesium sulfate, filtered, and concentrated under reduced pressure. Purification of the residue by flash chromatography on a silica gel column (330 g) using a gradient of 5-20% ethyl acetate in hexane gave the desired product (Rf=0.5, 10% EtOAc/hexane). ¹H NMR (399 MHz, CDCl₃): δ: 7.36-7.23 (m, 5H), 6.40 (d, J=6.07 Hz, 1H), 4.68 (s, 1H), 4.59 (q, J=6.06 Hz, 2H), 4.05-3.98 (m, 2H), 3.62-3.44 (m, 1H), 2.14-2.03 (m, 1H), 1.96 (d, J=17.25 Hz, 1H), 1.89-1.81 (m, 1H), 1.76-1.62 (m, 1H). ES MS=205.1 (M+1).

### Step 2: 6-[(benzyl oxy)methyl]tetrahydro-2H-pyran-3-ol

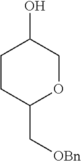

A stirred solution of 2-[(benzyloxy)methyl]-3,4-dihydro-2H-pyran (17 g, 83 mmol) in dry THF (200 mL) was cooled in ice bath. A solution of 9-BBN in THF (200 mL, 0.5 M solution) was added dropwise over 30 minutes and the stirred reaction was allowed to warm to room temperature over 18 hours. A solution of sodium perborate (50 g) in water (200 mL) was added slowly to quench excess 9-BBN, and the resulting mixture was stirred for 1 hour. The product was extracted with ether (3×200 mL). The organic layers were combined and washed with water and brine solution. The organic layer was dried over anhydrous magnesium sulfate, filtered, and concentrated under reduced pressure. Purification of the residue by flash chromatography on a silica gel column (330 g) using a gradient elution of 15-50% ethyl acetate in hexane gave the desired product (Rf=0.5, 40% EtOAc/hexane). ES MS=205.1 (M+1).

### Step 3: 6-[(benzyloxy)methyl]dihydro-2H-pyran-3(4H)-one

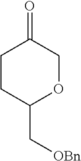

To a solution of 6-[(benzyloxy)methyl]tetrahydro-2H-pyran-3-ol (13 g, 59 mmol) in dry dichloromethane (200 mL) was added 1,1,1-triacetoxy-1,1-dihydro-1,2-benziodoxol-3(1H)-one (32 g, 76 mmol) and the reaction was stirred for 18 hours. The reaction was quenched with isopropyl alcohol (20 mL) and the solvents were removed under reduced pressure. The residue was suspended in ether (300 mL) and the solid was removed by filtration. The filtrate was concentrated under reduced pressure and the residue was purified by flash chromatography on a silica gel column (330 g) using a gradient elution of 15-50% ethyl acetate in hexane gradient to give the desired product (Rf=0.5, 25% EtOAc/hexane). ¹H NMR (399 MHz, CDCl₃): δ 7.38-7.33 (m, 4H); 7.33-7.27 (m, 1H); 4.60 (q, J=6.2 Hz, 2H); 4.20 (d, J=16.6 Hz, 2H); 4.00 (d, J=16.6 Hz, 1H); 3.63-3.49 (m, 2H); 2.62 (ddd, J=10.8, 10.6, 5.3 Hz, 1H); 2.47 (ddd, J=16.8, 10.9, 6.9 Hz, 1H); 2.12-1.88 (m, 2H). ES MS=203.3 (M+1).

### Step 4: tert-butyl {6-trans-[(benzyloxy)methyl]-3-cyanotetrahydro-2H-pyran-3-yl}methylcarbamate

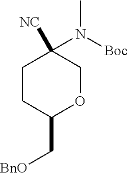

To a stirred solution of 6-[(benzyloxy)methyl]dihydro-2H-pyran-3(4H)-one (10 g, 45 mmol) in 1:1 methanol:water (100 mL) was added methylamine hydrochloride (4.6 g, 68 mmol) and sodium cyanide (3.4 g, 68 mmol). The solution was stirred for 48 hours at room temperature. The solution was made basic (pH=9) with saturated sodium carbonate solution (50 mL). The product was extracted into ethyl acetate (3×200 mL). The ethyl acetate layers were combined, washed with brine (100 mL), and dried over anhydrous magnesium sulfate. The solvent was removed under reduced pressure. The residue was dissolved in dichloromethane (300 mL) and to the stirred solution was added di-tert-butyl dicarbonate (10 g, 47 mmol). The solution was heated to 60° C. in a closed vessel and stirred for 36 hours, cooled to room temperature and then acidified with aqueous hydrochloric acid (50 mL of a 1M solution). The organic layer was separated, washed with water (50 mL) and brine solution (50 mL), dried over magnesium sulfate, filtered, and the solvent was removed under reduced pressure. Purification of the residue by flash chromatography on a silica gel column (750 g) using a gradient elution of 5-50% ethyl acetate in hexane gradient gave the desired product (Rf=0.5, 40% EtOAc/hexane). ¹H NMR (599 MHz, CDCl₃): δ 7.36-7.32 (m, 5H); 4.62-4.53 (m, 2H); 3.61-3.53 (m, 2H); 3.48-3.42 (m, 1H); 3.40 (d, J=11.0 Hz, 2H); 2.94 (s, 3H); 2.50-2.46 (m, 1H); 2.02-1.99 (m, 1H); 1.90-1.83 (m, 2H); 1.52 (s, 9H). ES MS=360.1 (M+1).

### Step 5: tert-Butyl {-3-[(E/Z)-amino(hydroxyimino)methyl]-6-[trans-(benzyloxy)methyl]tetrahydro-2H-pyran-3-yl}methylcarbamate

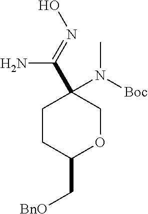

To a solution of tert-butyl {6-trans-[(benzyloxy)methyl]-3-cyanotetrahydro-2H-pyran-3-yl}methylcarbamate (4 g, 11 mmol) in methanol (80 mL) was added a 50% aqueous solution of hydroxylamine (1.5 mL, 22 mmol) and the mixture was stirred at 60° C. for 18 hours. The solution was concentrated under reduced pressure. The residue was dissolved in toluene and concentrated under reduced pressure (2×50 mL) to remove traces of hydroxylamine and water. The crude product was used without purification in the next step: ES MS=394.1 (M+1).

### Step 6: Diethyl (2E/Z)-2-{[((1E/Z)-amino {trans-6-[(benzyloxy)methyl]-3-[(tert-butoxycarbonyl)(methyl)amino]tetrahydro-2H-pyran-3-yl}methylene)amino]oxy}but-2-enedioate

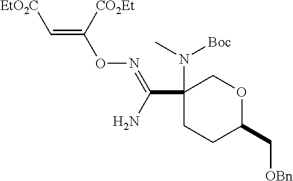

To a stirred solution of tert-butyl {(3R,6R)-3-[(E/Z)-amino(hydroxyimino)methyl]-6-[(benzyloxy)methyl]tetrahydro-2H-pyran-3-yl}methylcarbamate (4.3 g, 11 mmol) in methanol (20 mL) under nitrogen at −20° C. was added dimethyl acetylenedicarboxylate (2.0 mL, 15.3 mmol). The reaction was stirred at −20° C. for 2 hours and then allowed to warm to room temperature with stirring for 18 hours. The solvent was removed under reduced pressure. The residue was dissolved in toluene and concentrated under reduced pressure (50 mL) to remove traces of methanol. The crude product was used without purification in the next step: ES MS=536.2 (M+1).

### Step 7: Methyl 2-{6-trans-[(benzyloxy)methyl]-3-[(tert-butoxycarbonyl)(methyl)amino]tetrahydro-2H-pyran-3-yl}-5-hydroxy-6-oxo-1,6-dihydropyrimidine-4-carboxylate

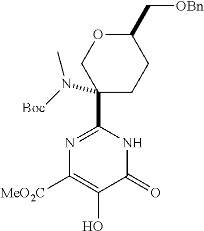

A stirred solution of diethyl (2E/Z)-2-{[((E/Z)-amino {6-trans-[(benzyloxy)methyl]-3-[(tert-butoxycarbonyl)(methyl)amino]tetrahydro-2H-pyran-3-yl}methylene)amino]oxy}but-2-enedioate (4 g, 7.5 mmol) in o-xylene (50 mL) under nitrogen was heated at 130° C. for 24 hours. The solution was cooled and the solvent was removed under reduced pressure. The residue was purified on a silica gel column (300 g) using a gradient elution of 0-10% MeOH in DCM. The product eluted at 6% MeOH in DCM: ES MS=504.1 (M+1).

### Step 8: methyl 2-{6-trans-[hydroxymethyl]-3-[(tert-butoxycarbonyl)(methyl)amino]tetrahydro-2H-pyran-3-yl}-5-hydroxy-6-oxo-1,6-dihydropyrimidine-4-carboxylate

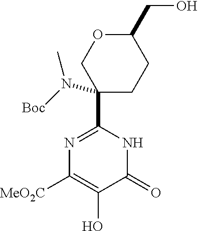

Under nitrogen atmosphere, methyl 2-{6-trans-[(benzyloxy)methyl]-3-[(tert-butoxycarbonyl)(methyl)amino]tetrahydro-2H-pyran-3-yl}-5-hydroxy-6-oxo-1,6-dihydropyrimidine-4-carboxylate (4.0 g, 8 mmol), ethanol (50 mL), and acetic acid (5 mL, 87 mmol) were combined. 10% Pd/C (1 g) was added and the mixture was shaken on a Parr apparatus under an atmosphere of hydrogen gas at 50 psi for 48 hours. The mixture was filtered through diatomaceous earth to remove catalyst and the filtrate solvents were removed under reduced pressure. The residue was dissolved in toluene (100 mL) and concentrated under reduced pressure to remove traces of ethanol and water. The crude product was used without purification in the next step: ES MS=414.3 (M+1).

### Step 9: methyl 1-[(tert-butoxycarbonyl)(methyl)amino]-5-[(methylsulfonyl)oxy]-6-oxo-3,7-diazatricyclo[7.2.2.0^{2,7}]trideca-2,4-diene-4-carboxylate

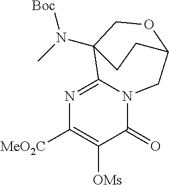

Methyl 2-{6-trans-[hydroxymethyl]-3-[(tert-butoxycarbonyl)(methyl)amino]tetrahydro-2H-pyran-3-yl}-5-hydroxy-6-oxo-1,6-dihydropyrimidine-4-carboxylate (2.0 g, 4.75 mmol) was dissolved in dry DCM (50 mL) under nitrogen and the stirred solution was cooled in an ice bath. To the mixture was added triethylamine (3.37 mL, 24.2 mmol) followed by methanesulfonyl chloride (1.5 mL, 19.35 mmol). The mixture was stirred for 1 hour and then diluted with water (20 mL). The organic layer was separated, washed with brine solution (20 mL), dried over anhydrous sodium sulfate, filtered, and concentrated. The crude trismesylate was used without further purification. ES MS: m/z=648.1 (M+1).

Cesium carbonate (3.32 g, 10.19 mmol) was added to a stirred solution of the trismesylate (3.0 g, 4.63 mmol) in dimethylformamide (40 mL). The reaction mixture was placed in an oil bath preheated to 90° C. and stirred for 20 minutes. The solution was cooled, diluted with ethyl acetate, and filtered. The filtrate was concentrated under reduced pressure. The residue was purified by flash chromatography on a silica gel column (40 g) using a gradient elution of 30-100% ethyl acetate in hexane. The product eluted at 70% ethyl acetate in hexane. The two enantiomers were separated by chiral chromatography utilizing chiral AS-H column (5 μm, 21.2 mm×25 cm) with 10% EtOH in CO₂ under isocratic for 10 minutes, 100 bar, 35° C. ¹H NMR (599 MHz, CDCl₃): δ 5.13 (d, J=12.3 Hz, 1H); 4.67 (d, J=15.9 Hz, 1H); 4.52-4.45 (m, 1H); 4.12-4.00 (m, 1H); 3.93 (m, 4H); 3.52 (s, 3H); 3.03 (s, 3H); 2.40-2.26 (m, 2H); 2.23-2.14 (m, 2H); 1.40 (s, 9H), ES MS: m/z=474.1 (M+1).

### Step 10: tert-Butyl (4-{[(4-fluorobenzyl)amino]carbonyl}-5-hydroxy-6-oxo-10-oxa-3,7-diazatricyclo[7.2.2.0^{2,7}]trideca-2,4-dien-1-yl)methylcarbamate

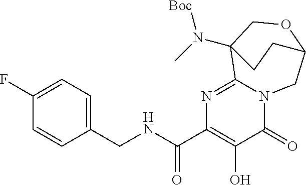

To a solution of methyl 1-[(tert-butoxycarbonyl)(methyl)amino]-5-[(methylsulfonyl)oxy]-6-oxo-3,7-diazatricyclo[7.2.2.0^{2,7}]trideca-2,4-diene-4-carboxylate (1st eluting enantiomer from Step 9, 150 mg, 0.37 mmol) in ethanol (10 mL) was added 4-fluorobenzylamine (0.13 mL, 0.91 mmol). The stirred solution was heated to 80° C. for 18 hours. The solution was cooled and the ethanol was removed under reduced pressure. The crude product was dissolved in ethyl acetate (50 mL) and washed with aqueous hydrochloric acid (10 mL of a 1.0 M solution). The organic layer was separated, washed successively with water and brine, dried over anhydrous magnesium sulfate, and the solvent was removed under reduced pressure. The crude product was used without further purification. ES MS: m/z=489.3 (M+1)

### Step 11: N-(4-fluorobenzyl)-5-hydroxy-1-(methylamino)-6-oxo-10-oxa-3,7-diazatricyclo[7.2.2.0^{2,7}]trideca-2,4-diene-4-carboxamide Hydrochloride

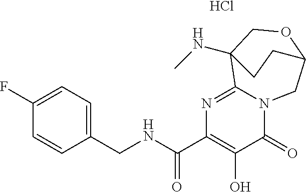

tert-Butyl (4-{[(4-fluorobenzyl)amino]carbonyl}-5-hydroxy-6-oxo-10-oxa-3,7-diazatricyclo[7.2.2.0^{2,7}]trideca-2,4-dien-1-yl)methylcarbamate (150 mg, 0.37 mmol) was dissolved in HCl/dioxane (4 mL of a 4 M solution) and stirred for 3 hours. The solution was concentrated under reduced pressure. The residue was suspended in toluene (20 mL) and concentrated under reduced pressure to remove traces of water. The crude product was dried under high vacuum and used without purification in the next step: ES MS: m/z=389.2 (M+1)

### Step 12: N-(4-{[(4-Fluorobenzyl)amino]carbonyl)}-5-hydroxy-6-oxo-10-oxa-3,7-diazatricyclo[7.2.2.0^{2,7}]trideca-2,4-dien-1-yl)-N′,N′-trimethylethanediamide

To a stirred solution of N-(4-fluorobenzyl)-5-hydroxy-1-(methylamino)-6-oxo-10-oxa-3,7-diazatricyclo[7.2.2.0^{2,7}]trideca-2,4-diene-4-carboxamide hydrochloride (134 mg, 0.35 mmol) in dry DCM (5 mL) under nitrogen was added triethylamine (194 μL, 1.4 mmol) followed by ethyl chlorooxalate (40 μL, 0.5 mmol). The reaction was stirred at room temperature for 2 hours and concentrated under reduced pressure. The residue was dissolved in methanol containing dimethylamine (5 mL of a 2 M solution) and the mixture was heated at 60° C. for 18 hours. The solution was concentrated under reduced pressure and the crude product was purified by reverse phase HPLC (Xterra C18 column) using a water:acetonitrile containing 0.1% TFA mobile phase gradient (20-70% acetonitrile over 30 minutes, 50 mL/minute). Concentration of product containing fractions gave the desired product as an amorphous white solid: ¹H NMR (399 MHz, DMSO): δ 9.58 (br. s, 1H); 7.38 (dd, J=8.2, 5.6 Hz, 2H); 7.15 (dd, J=8.3, 5.8 Hz, 2H); 4.98 (d, J=12.0 Hz, 1H); 4.63 (dd, J=16.2, 5.7 Hz, 1H); 4.52 (dd, J=15.0, 6.6 Hz, 1H); 4.45 (m, 2H); 4.40 (d, J=6.7 Hz, 1H); 4.03-3.89 (m, 2H); 2.96 (s, 3H); 2.90 (s, 6 H); 2.21-2.13 (m, 3H); 1.55-1.46 (m, 1H). HR MS: ESI=488.1953 (M+1); calculated: 488.1946 (M+1).

### Example 10B

### N-(4-{[(4-Fluorobenzyl)amino]carbonyl}-5-hydroxy-6-oxo-10-oxa-3,7-diazatricyclo[7.2.2.0^{2,7}]trideca-2,4-dien-1-yl)-N′,N′,N′-trimethylethanediamide

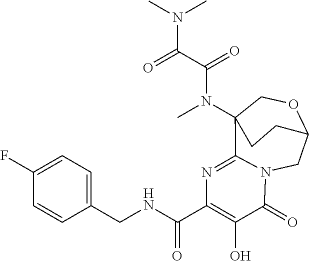

The 2nd eluting enantiomer from Step 9 of Example 10A was converted to the title compound using the procedures given in Steps 10-12 for Example 10A. ¹H NMR (399 MHz, CDCl₃): δ 9.57 (br. s, 1H); 7.37 (dd, J=8.2, 5.4 Hz, 2H); 6.99 (dd, J=16.5, 8.4 Hz, 2 H); 5.19 (d, J=12.3 Hz, 1H); 4.92 (dd, J=16.3, 5.8 Hz, 1H); 4.62 (dd, J=14.4, 6.7 Hz, 1H); 4.53-4.42 (m, 2H); 4.01-3.92 (m, 2H); 3.06-2.96 (m, 9H); 2.44-2.30 (m, 1H); 2.30-2.13 (m, 2H); 1.55-1.41 (m, 1H). HR MS: ESI=488.1940 (M+1); calculated 488.1946 (M+1).

### Example 11A

### N-(4-{[(4-Fluorobenzyl)amino]carbonyl}-5-hydroxy-6-oxo-10-oxa-3,7-diazatricyclo[7.2.2.0^{2,7}]trideca-2,4-dien-1-yl)-N′,N′,N′-trimethylethanediamide

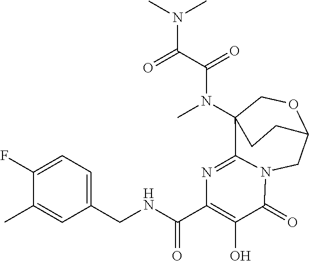

Starting with the 1^{st} eluting enantiomer from Step 9 of Example 10A, the title compound was prepared using the procedures Steps 10-12 of Example 10A except that 4-fluoro-3-methylbenzylamine was used in place of 4-fluorobenzylamine in Step 10. ¹H NMR (399 MHz, CDCl₃): δ 9.53 (br. s, 1H); 7.23-7.14 (m, 2H); 6.92 (t, J=9.0 Hz, 1H); 5.19 (d, J=12.3 Hz, 1H); 4.92 (dd, J=16.3, 5.8 Hz, 1H); 4.59 (dd, J=14.5, 6.7 Hz, 1H); 4.51 (dd, J=8.7, 5.7 Hz, 1H); 4.43 (dd, J=14.6, 6.1 Hz, 1H); 3.98 (dd, J=14.0, 7.8 Hz, 2H); 3.06-2.95 (m, 9H); 2.43-2.31 (m, 1H); 2.24 (s, 3H); 2.18 (d, J=11.5 Hz, 2H); 1.54-1.42 (m, 2H). HR MS: ESI=502.2091 (M+1); calculated 502.2096 (M+1).

### Example 11B

### N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′,N′-trimethylethanediamide

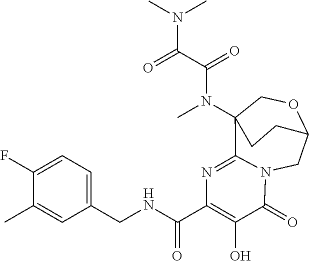

Starting with the 2^{nd} eluting enantiomer from Step 9 of Example 10A, the title compound was prepared using the procedures of Steps 10-12 of Example 10A except that 4-fluoro-3-methylbenzylamine was used in place of 4-fluorobenzylamine in Step 10. HR MS: ESI=502.2094 (M+1); calculated 502.2096 (M+1)

### Example 12A

### N-Ethyl-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′-dimethylethanediamide

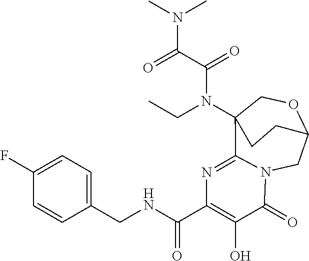

The title compound was prepared using the procedures given in Example 10A except that ethylamine hydrochloride was used in place of methylamine hydrochloride in Step 4. Separation of enantiomers in Step 9 was accomplished by chiral chromatography using SFC conditions. (50 mL/minute on a 5 μm, 21.2 mm×25 cm AS-H column, 10% EtOH in CO₂, isocratic for 10 minutes, 100 bar, 35° C.).

The 1^{st} eluting enantiomer from step 9 was further elaborated to the title compound as described in the procedures of Steps 10-12 of Example 10A. ¹H NMR (399 MHz, CDCl₃): δ 9.31 (s, 1H); 7.37 (dd, J=8.2, 5.3 Hz, 2H); 7.03-6.93 (m, 2H); 5.20 (dd, J=12.1, 1.4 Hz, 2H); 4.91 (dd, J=16.2, 5.5 Hz, 1H); 4.66 (dd, J=14.5, 6.9 Hz, 1H); 4.51 (dd, J=8.0, 5.1 Hz, 1H); 4.44 (dd, J=14.5, 5.9 Hz, 1H); 3.98 (d, J=16.3 Hz, 1H); 3.89 (d, J=12.2 Hz, 1H); 3.60 (dd, J=15.8, 7.4 Hz, 1H); 3.35 (dd, J=15.8, 7.5 Hz, 1H); 2.98 (d, J=2.3 Hz, 6H); 2.43-2.28 (m, 2H); 2.29-2.17 (m, 2H); 1.26 (t, J=7.0 Hz, 3H).

HR MS: ESI=502.295 (M+1); calculated 502.2096 (M+1).

### Example 12B

### N-Ethyl-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′-dimethylethanediamide

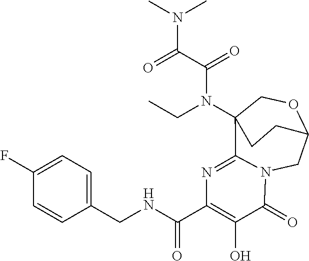

The title compound was prepared using the procedures given in Example 10A except that the 2^{nd} eluting enantiomer from Step 9, Example 12A was employed. HR MS: ESI=502.2096 (M+1); calculated 502.2096 (M+1).

### Example 13A

### N-(4-{[(4-fluorobenzyl)amino]carbonyl}-5-hydroxy-8-methyl-6-oxo-3,7-diazatricyclo[7.2.2.0^{2,7}]trideca-2,4-dien-1-yl)-N′,N′,N″-trimethylethanediamide

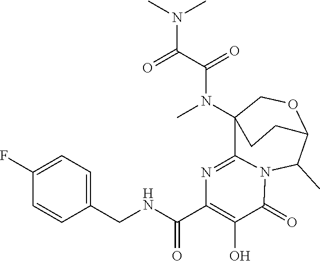

The title compound was synthesized from 4-[1-(benzyloxy)ethyl]cyclohexanone (prepared in accordance with J. Am. Chem. Soc. 1988, 110, p. 2312-14) using the procedures given in Example 10A, Steps 4-9. The two enantiomers in Step 9 were separated by chiral chromatography under SFC conditions (AS-H chiral column, 5 μm, 21.2 mm×25 cm, 10% EtOH in CO₂, isocratic for 10 minutes, 100 bar, 35° C.). The first eluting enantiomer from Step 9 was further elaborated as described in Steps 10-12 of Example 10A to give the title compound. HR MS: ESI=500.2304 (M+1); calculated 500.2325 (M+1).

### Example 13B

### N-(4-{[(4-fluorobenzyl)amino]carbonyl}-5-hydroxy-8-methyl-6-oxo-3,7-diazatricyclo[7.2.2.0^{2,7}]trideca-2,4-dien-1-yl)-N′,N′,N″-trimethylethanediamide

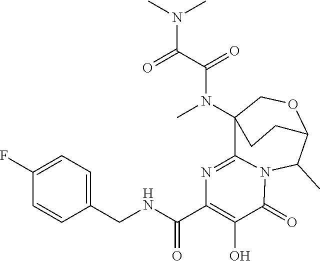

The title compound was synthesized using the procedures given in Example 10A, except that the second eluting enantiomer from Step 9 was utilized. HR MS: ESI=500.2306 (M+1); calculated 500.2304 (M+1).

### Example 14

### N-(4-{[(4-Fluoro-3-methylbenzyl)amino]carbonyl}-5-hydroxy-9-methoxy-6-oxo-3,7-diazatricyclo[7.2.2.0^{2,7}]trideca-2,4-dien-1-yl)-N,N′,N′-trimethylethanediamide

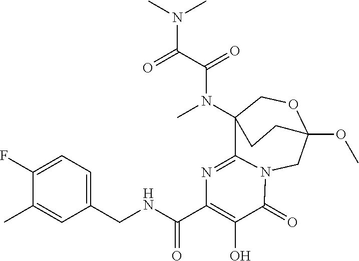

### Step 1: 4-[(Benzyloxy)methyl]-4-hydroxycyclohexanone

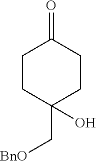

To a cold (0° C.) suspension of sodium hydride (60 wt % in mineral oil; 2.01 g, 50.3 mmol) in DMF (168 ml), benzyl alcohol (4.78 ml, 46.1 mmol) was added dropwise with the reaction temperature kept under 3° C. After the addition was complete, the mixture was stirred for 15 minutes at 0° C., then at room temperature for 45 minutes. The reaction mixture was cooled back to 0° C., and 1,7,10-trioxadispiro-[2.2.4.2]dodecane (7.14 g, 41.9 mmol (which was synthesized in accordance with the procedure in Synthetic Communications 2003, vol. 33, p. 2135-2143) was added with the reaction temperature kept under 5° C. The reaction was allowed to warm up to room temperature, and then heated overnight at 55° C. The reaction was cooled and poured into ice water (1300 mL) and EtOAc (250 mL). The aqueous layer was extracted three more times with EtOAc. The combined organic extracts were dried over Na₂SO₄, filtered and concentration under vacuum. The crude product was purified by flash column chromatography (RediSep ISCO column, 120 g silica) eluting with a 0-50% EtOAc/hexane stepwise gradient over 40 minutes. Collection and concentration of the appropriate fractions afforded 8-[(benzyloxy)methyl]-1,4-dioxaspiro[4.5]decan-8-ol as a colorless oil. ES MS=279.3 (M+1). This intermediate (4.93 g, 17.71 mmol) was stirred as a solution in a mixture of THF (44 mL) and aqueous HCl (18 mL) at room temperature overnight. The product mixture was concentrated under vacuum. The residue was partitioned between water and EtOAc. The organic extract was dried over Na₂SO₄, filtered and concentration under vacuum to provide the title compound as a pale yellow oil. This material was used in the next step without further purification. ES MS=235.3 (M+1).

### Step 2: tert-Butyl (4-{[(4-fluoro-3-methylbenzyl)amino]carbonyl}-5,9-dihydroxy-6-oxo-3,7-diazatricyclo[7.2.2.0^{2,7}]trideca-2,4-dien-1-yl)methylcarbamate

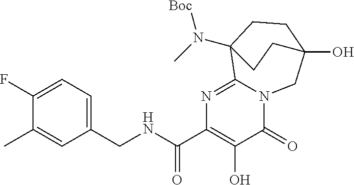

Following the procedure described in Example 1, Steps 1 to 7, and substituting 4-fluorobenzylamine with 4-fluoro-3-methylbenzylamine in Step 7, the title compound was prepared.

### Step 3: N-(4-{[(4-Fluoro-3-methylbenzyl)-5-hydroxy-9-methoxy-1-(methyl-amino)-6-oxo-3,7-diazatricyclo[7.2.2.0^{2,7}]trideca-2,4-diene-4-carboxamide

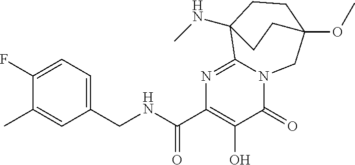

A cold (0° C.) solution of tert-butyl (4-{[(4-fluoro-3-methylbenzyl)-amino]carbonyl}-5,9-dihydroxy-6-oxo-3,7-diazatricyclo[7.2.2.027]trideca-2,4-dien-1-yl)methyl-carbamate (120 mg, 0.23 mmol) in anhydrous DMF (2 mL) was treated with NaH (37 mg, 60% oil dispersion), stirred for 10 minutes, treated with dimethyl sulfate (88 mg, 0.69 mmol), and stirred at the same temperature for 3 hours. The reaction mixture was quenched with aqueous HCl and extracted with ethyl acetate. The organic extract was washed with brine, dried over sodium sulfate, filtered, and concentrated under vacuum. LC-MS analysis of the residue indicated a mixture of mono- and dimethylated product was produced. The residue was dissolved in methylene chloride (2 mL), cooled to 0° C., and treated with boron tribromide (0.67 mL, 1 M solution in methylene chloride). The mixture was stirred at 0° C. for 2 hours, and room temperature for 1 hour. The product mixture was treated with methanol and concentrated under vacuum to provide the titled compound. ES MS=431.2 (M+1).

### Step 4: N-(4-{[(4-Fluoro-3-methylbenzyl)amino]carbonyl}-5-hydroxy-9-methoxy-6-oxo-3,7-diazatricyclo[7.2.2.0^{2,7}]trideca-2,4-dien-1-yl)-N,N′,N′-trimethylethanediamide

Following the procedure as described in Example 1, Step 9, the oxalylamide moiety was installed, and the title compound was prepared. ¹H NMR (400 MHz, CDCl₃): δ 9.55 (br. s, 1H); 7.52-7.17 (m, 2H); 6.91 (t, J=8.4 Hz, 1H); 4.92 (d, J=14.7 Hz, 1H); 4.50 (dd, J=14.2, 6.9 Hz, 1H); 4.42 (dd, J=15.2, 5.4 Hz, 1H); 3.59 (d, J=15.7 Hz, 1H); 3.37-3.31 (m, 1H); 3.29 (s, 3H); 3.02 (s, 3H); 2.99 (s, 3H); 2.98 (s, 3H); 2.23 (s, 3H); 2.19-2.16 (m, 2H); 1.98-1.93 (m, 2H); 1.68-1.65 (m, 1H). HR MS: ESI=530.2408 (M+1); calculated 530.2415 (M+1).

### Example 15

### N-(4-{[(4-Fluorobenzyl)amino]carbonyl}-5-hydroxy-9-methoxy-6-oxo-3,7-diazatricyclo[7.2.2.0^{2,7}]trideca-2,4-dien-1-yl)-N,N′,N′-trimethylethanediamide

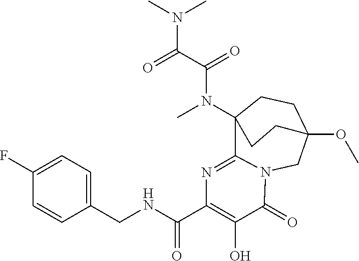

The title compound was synthesized using the procedures given in Example 14 except that 4-fluorobenzylamine was used in place of 4-fluoro-3-methylbenzylamine in Step 2.

HR MS: ESI=516.2263 (M+1); calculated 516.2258 (M+1).

### Example 16

### N-Ethyl-N-(4-{[(4-fluorobenzyl)amino]carbonyl}-5-hydroxy-9-methoxy-6-oxo-3,7-diazatricyclo[7.2.2.0^{2,7}]trideca-2,4-dien-1-yl)-N,N′-dimethylethanediamide

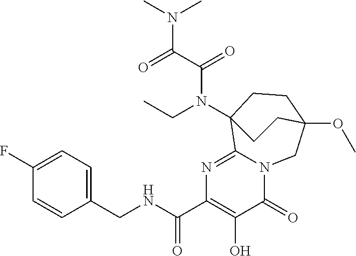

The title compound was synthesized using the procedures given in Example 14 except that ethylamine was used in place of methylamine in the Streker reaction. HR MS: ESI 530.2416 (M+1); calculated 530.2415 (M+1).

### Example 17

### N-(4-{[(4-Fluorobenzyl)amino]carbonyl}-5,9-dihydroxy-6-oxo-3,7-diazatricyclo[7.2.2.0^{2,7}]trideca-2,4-dien-1-yl)-N,N′,N′-trimethylethanediamide

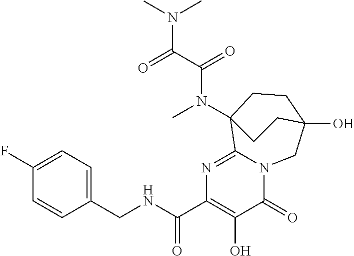

The title compound was synthesized using the procedures given in Example 14 with the exclusion of Step 3, O-methylation. HR MS: ESI=502.2107 (M+1); calculated 502.2102 (M+1).

### Example 18

### Enantiomers of N-ethyl-N-(4-{[(4-fluoro-3-methylbenzyl)amino]carbonyl}-5-hydroxy-6-oxo-10-oxa-3,7-diazatricyclo[7.2.2.0^{2,7}]trideca-2,4-dien-1-yl)-N,N-dimethylethanediamide (Analog of Example 10)

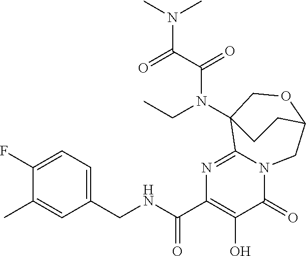

The title compound was synthesized using the procedures given in Example 10A with the following modifications:
1. Ethylamine hydrochloride was used in place of methylamine hydrochloride in Step 4.
2. After separation of the racemic intermediate (methyl 1-[(tert-butoxycarbonyl)(ethyl)amino]-5-[(methylsulfonyl)oxy]-6-oxo-10-oxa-3,7-diazatricyclo[7.2.2.0^{2,7}]trideca-2,4-diene-4-carboxylate) into individual enantiomers by chiral chromatography in Step 9.

Compound 18A: The faster enantiomer was treated with 4-fluoro-3-methylbenzylamine in place of 4-fluorobenzylamine as described in Example 10A Step 10.

HR MS: ESI=516.2266 (M+1); calculated 516.2258 (M+1).

Compound 18B: The second eluting enantiomer of methyl 1-[(tert-butoxycarbonyl)(ethyl)amino]-5-[(methylsulfonyl)oxy]-6-oxo-10-oxa-3,7-diazatricyclo[7.2.2.0^{2,7}]trideca-2,4-diene-4-carboxylate was converted to title compound. HR MS: ESI=516.2269 (M+1); calculated 516.2258 (M+1).

### Example 19

### N-(4-{[(4-fluorolbenzyl)amino]carbonyl}-5-hydroxy-6-oxo-10-oxa-3,7-diazatricyclo[7.2.2.0^{2,7}]trideca-2,4-dien-1-yl)-N′,N′-dimethyl-N-propylethanediamide (Analog of Example 10)

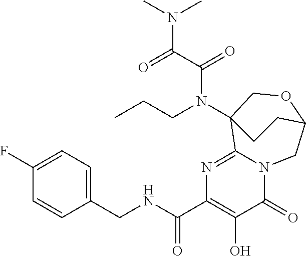

The title compound was synthesized using the procedures given in Example 10A replacing methylamine hydrochloride with n-propylamine hydrochloride in Step 4. HR MS: ESI=516.2252 (M+1); calculated 516.2258 (M+1).

### Example 20

Enantiomers of N-(9-ethyl-4-{[(4-fluorolbenzyl)amino]carbonyl}-5-hydroxy-6-oxo-10-oxa-3,7-diazatricyclo[7.2.2.0^{2,7}]trideca-2,4-dien-1-yl)-N,N′,N′-trimethylethanediamide

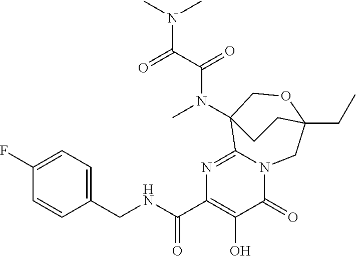

### Step 1: tert-Butyl[(2-ethyl-3,4-dihydro-2H-pyran-2-yl)methoxy]dimethylsilane

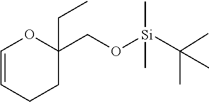

To a cold (−78° C.) solution of ethyl 3,4-dihydro-2H-pyran-2-carboxylate (2 g, 12.8 mmol) in a mixture of anhydrous THF (50 mL) and HMPA (3.34 mL), a solution of LDA (11.1 mL, 16.6 mmol; 1.5 M in cyclohexane) was added. The reaction mixture was stirred at −78° C. for 1 hour, treated with ethyl iodide (5.17 mL, 64 mmol), and allowed to warn up to room temperature. The reaction mixture was quenched with saturated aqueous ammonium chloride and diluted with ethyl acetate. The organic extract was washed with brine, dried over sodium sulfate, filtered, and concentrated under vacuum. The residue was subjected to column chromatography on silica gel eluting with a 0% to 40% ethyl acetate/hexane gradient. Collection and concentration of appropriate fractions afforded ethyl 2-ethyl-3,4-dihydro-2H-pyran-2-carboxylate. The ester was reduced to the corresponding alcohol with LAH. A cold (0° C.) solution of the above ester (9 g, 48.9 mmol) in anhydrous ether was treated dropwise with a solution of LAH (12.1 mL, 48.9 mmol; 4 M solution in THF/toluene). The reacting mixture was stirred at the same temperature for 3 hours, quenched sequentially with water (1.9 mL), 10% aqueous NaOH (19 mL), and saturated ammonium chloride. The product mixture was filtered through a pad of Celite, and the filtrate was concentrated under vacuum to provide the corresponding alcohol, which was silylated without further purification. A solution of the above alcohol (6.5 g, 45.7 mmol), DMAP (0.56 g, 4.6 mmol), imidazole (4.05 g, 59.4 mmol), and tert-butyldimethylchlorosilane (8.3 g, 54.9 mmol) in DMF was stirred at room temperature overnight. The reaction mixture was concentrated under vacuum, and the residue dissolved in ethyl acetate. The product solution was washed with water, brine, dried over sodium sulfate, filtered, and concentrated. The residue was subjected to column chromatography on silica gel eluting with a 0% to 10% ethyl acetate/hexane gradient. Collection and concentration of appropriate fractions afforded tert-butyl[(2-ethyl-3,4-dihydro-2H-pyran-2-yl)methoxy]dimethylsilane. ¹H NMR (400 MHz, CDCl₃) δ 6.27 (dt, J=6.2, 1.9 Hz, 1H), 4.61 (m, 1H), 3.54 (d, J=10 Hz, 1H), 3.50 (d, J=10 Hz, 1H), 1.97-1.93 (m, 2H), 1.78-1.53 (m, 5H), 1.28 (br signal, 2H), 0.89 (br s), 0.02 (s, 6 H).

### Step 2: 6-({[tert-Butyl(dimethyl)silyl]oxy}methyl)-6-ethyldihydro-2H-pyran-3-(4H)-one

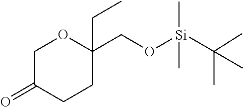

To a cold (0° C.) solution of tert-butyl[(2-ethyl-3,4-dihydro-2H-pyran-2-yl)methoxy]dimethylsilane (1 g, 3.9 mmol) in THF (40 mL), borane dimethyl sulfide complex (0.39 mL, 3.9 mmol) was added dropwise. The reaction mixture was stirred at room temperature for 4 hours, cooled back to 0° C. and treated sequentially with hydrogen peroxide (1.13 mL, 12.8 mmol; 30% aqueous solution), and aqueous sodium hydroxide (0.27 mL, 5.1 mmol; 50% solution). The reaction mixture was diluted with ethyl ether. The organic layer was washed with water, brine, dried over sodium sulfate, filtered, and concentrated under vacuum. The residue was subjected to column chromatography on silica gel eluting with a 0% to 50% ethyl acetate/hexane gradient. Collection and concentration of appropriate fractions afforded 6-({[tert-butyl(dimethyl)silyl]oxy}-methyl)-6-ethyltetrahydro-2H-pyran-3-ol which was oxidized to the corresponding ketone as follows:

To a cold (−78° C.) solution of the alcohol (19.0 g, 69.2 mmol) and DMSO (14.7 mL; 208 mmol) in dichloromethane (500 mL), a solution of oxalyl chloride (9.1 mL; 104 mmol) in dichloromethane (100 mL) was added dropwise. After the addition was complete, the reacting mixture was stirred at −78° C. for half an hour, and treated with triethylamine (48.2 mL, 346 mmol). The resultant mixture was stirred at −78° C. for ˜30 minutes, 0° C. for 2 hours. The product mixture was quenched with saturated aqueous ammonium chloride. The organic layer was washed with brine, dried over sodium sulfate, filtered and concentrated under vacuum. A mixture of the title compound and the corresponding desilylated product was obtained.

The mixture of title compound and desilylated product was resilylated in a manner similar to the procedure described in Example 20, Step 1 to afford the title compound as the major product. ¹H NMR (400 MHz, CDCl₃) δ 4.11 (d, J=17.9 Hz, 1H), 4.00 (d, J=17.9 Hz, 1H), 3.57 (s, 2H), 2.59-2.38 (m, 2H), 2.13 (m, 1H), 1.78-1.51 (m, 3H), 1.27 (br s, 3H), 0.90 (s, 9H), 0.07 (s, 6H).

### Step 3: N-(9-Ethyl-4-{[(4-fluorolbenzyl)amino]carbonyl}-5-hydroxy-6-oxo-10-oxa-3,7-diazatricyclo[7.2.2.0^{2,7}]trideca-2,4-dien-1-yl)-N,N′,N′-trimethylethanediamide

Following the procedure described in Example 5, Steps 4 to 14, the title compound was prepared with the following modifications:
1. In Step 4, methylamine hydrogen chloride was used in place of ammonium chloride.
2. In Step 14, chiral column chromatography separation of the racemic final product provided the faster eluting enantiomer (Compound 20A) and the slower eluting isomer (Compound 20B).

Compound 20A: ¹H NMR (400 MHz, CDCl₃): δ 9.55 (br s, 1H), 7.37 (dd, J=8.2, 5.4 Hz, 2H), 6.98 (t, J=8.52 Hz, 2H), 5.15 (d, J=12.4 Hz, 1H), 4.79 (d, J=16.1 Hz, 1 H), 4.62 (dd, J=14.5, 6.7 Hz, 1H), 4.47 (dd, J=14.5, 6.1 Hz, 1H), 3.96 (d, J=12.4 Hz, 1H), 3.78 (d, J=16.1 Hz), 3.02 (s, 3H), 3.00 (s, 3H), 2.97 (s, 3H), 2.27-1.98 (m, 2H), 1.73-1.62 (m, 2H), 1.46 (m, 1H), 1.00 (t, J=7.4 Hz, 3H). HR MS: ESI=516.2268 (M+1); calculated 516.2258 (M+1).

Compound 20B: ¹HR MS: ESI=516.2270 (M+1); calculated 516.2258 (M+1).

### Example 21

Enantiomers of N-4-{[(4-fluorolbenzyl)amino]carbonyl}-5-hydroxy-9-methyl-6-oxo-10-oxa-3,7-diazatricyclo[7.2.2.0^{2,7}]trideca-2,4-dien-1-yl)-N,N′,N′-trimethylethanediamide

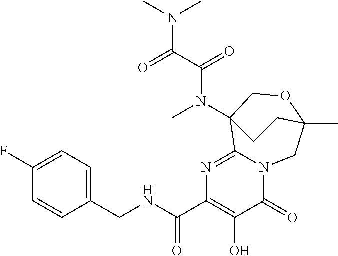

Following the procedure described in Example 20, the title compound was prepared except in Step 1, iodomethane was used in place of iodoethane. Chiral column chromatography separation of the racemic final product provided a faster eluting enantiomer (Compound 21A) and a slower eluting isomer (Compound 21B).

Compound 21A: HR MS: ESI=502.2112 (M+1); calculated 502.2102 (M+1)

Compound 21B: HR MS: ESI=502.2116 (M+1); calculated 502.2102 (M+1).

### Example 22

Enantiomers of N′-(9-ethyl-4-{[(4-fluorolbenzyl)amino]carbonyl}-5-hydroxy-6-oxo-10-oxa-3,7-diazatricyclo[7.2.2.0^{2,7}]trideca-2,4-dien-1-yl)-N,N-dimethylethanediamide

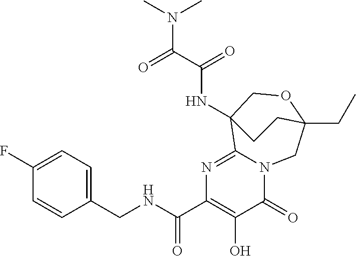

Following the procedure described in Example 20, the title compound was prepared except in Step 4, ammonium chloride was used in place of methylamine hydrogen chloride. Chiral column chromatography separation of the racemic final product in provided a faster eluting enantiomer (Compound 22A) and a slower eluting isomer (Compound 22B).

Compound 22A: HR MS: ESI=502.2109 (M+1); calculated 502.2102 (M+1).

Compound 22B: HR MS: ESI=502.2118 (M+1); calculated 502.2102 (M+1).

### Example 23

Enantiomers of N-5-{[(4-fluorolbenzyl)amino]carbonyl}-4-hydroxy-3-oxo-10-oxa-2,6-diazatricyclo[6.3.2.0^{2,7}]trideca-4,6-dien-8-yl)-N,N′,N′-trimethylethanediamide

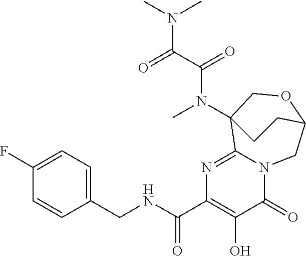

### Step 1: 3-(Benzyloxy)-2,3,4,5-tetrahydrooxepine

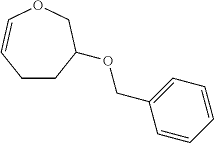

To a cold (0° C.) solution of allyl glycidyl ether (24 g, 210 mmol) and copper (I) iodide (4 g, 21 mmol) in anhydrous THF (500 mL), a solution of vinyl magnesium bromide (300 mL, 210 mmol; 7 M) was added dropwise. After the addition was complete, the reaction mixture was stirred at 0° C. for 1 hour and quenched with saturated aqueous ammonium chloride. The aqueous layer was extracted twice with ethyl acetate. The combined organic extracts were washed, dried over sodium sulfate, filtered, and concentrated under vacuum to afford the intermediate 1-(allyloxy)hex-5-en-2-ol. This intermediate was benzylated as follows and used without further purification:

To a cold (0° C.) solution of the above alcohol (30.0 g, 211 mmol) in anhydrous DMF, sodium hydride (8.4 g, 211 mmol; 60% dispersion in oil) was added. The reaction mixture was stirred at the same temperature for ˜10 minutes and treated with benzyl bromide (36.1 g, 211 mmol). The mixture was stirred at room temperature for 2 days, quenched with water, and diluted with ethyl acetate. The organic phase was washed sequentially with water and brine, and then dried over anhydrous sodium sulfate, filtered, and concentrated under vacuum. The residue was subjected to column chromatography on silica gel eluting with a 0% to 5% ethyl acetate/hexane gradient. Collection and concentration of appropriate fractions afforded intermediate 1-(allyloxy)hex-5-en-2-yl benzyl ether. ¹H NMR (400 MHz, CDCl₃): δ 7.38-7.29 (m, 5H); 5.97-5.79 (m, 2H); 5.30 (d, J=1.9 Hz, 1H); 5.26 (d, J=1.9 Hz, 1H); 5.18 (d, J=10.5 Hz, 1H); 5.15-5.01 (m, 2H); 4.73-4.57 (m, 2H); 4.51 (d, J=3.4 Hz, 1H); 4.02-3.99 (m, 2H); 3.65 (t, J=5.7 Hz, 1H); 3.53-3.49 (m, 2H); 2.39-2.33 (m, 2H).

A solution of the intermediate bis-olefin (35 g, 151 mmol) in toluene (700 mL) was treated with Grubbs catalyst, 1st generation[6.0 g, benzylidene-bis(tricyclohexylphosphine)dichlororuthenium]. After stirring at room temperature overnight, an additional 6 g of the catalyst was added and the reaction mixture was stirred at the same temperature for two more days. The resultant RCM product was isomerized in the same pot (Chem. Eur. J. 2008, 14, 6135-6141) as described in the following. The RCM reaction mixture was treated with granulated sodium hydroxide (9 g, 223 mmol) and isopropyl alcohol (150 mL), heated under reflux for 1 hour, and concentrated under vacuum. The residue was diluted with ethyl acetate and water. The organic phase was washed with water and then brine, dried over sodium sulfate, filtered and concentrated under vacuum. The residue was subjected to column chromatography on silica gel eluting with a 0% to 20% ethyl acetate/hexane gradient. Collection and concentration of appropriate fractions afforded title compound 3-(benzyloxy)-2,3,4,5-tetrahydrooxepin. ES MS 205.2 (M+1).

### Step 2: 6-(Benzyloxy)oxepan-3-one

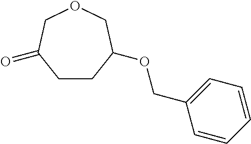

To a cold (0° C.) solution of 3-(benzyloxy)-2,3,4,5-tetrahydrooxepin (13 g, 63.6 mmol) in THF (260 mL), borane dimethyl sulfide complex (3.1 mL, 31.8 mmol) was added dropwise. The reaction mixture was stirred at room temperature for 4 hours, cooled back to 0° C., and treated with sodium perborate monohydrate (19 g, 200 mmol) and water (65 mL). The reaction mixture was stirred at room temperature overnight and diluted with ethyl acetate. The organic layer was washed with water, brine, dried over sodium sulfate, filtered, and concentrated under vacuum. The residue was subjected to column chromatography on silica gel eluting with a 0% to 70% ethyl acetate/hexane gradient. Collection and concentration of appropriate fractions afforded 6-(benzyloxy)-oxepan-3-ol which was oxidized to the corresponding ketone as follows:

To a cold (−78° C.) solution of DMSO (5.8 mL; 81 mmol) in dichloromethane (100 mL), oxalyl chloride (17.6 mL; 35.1 mmol) was added dropwise. After the reaction mixture was stirred at −78° C. for ˜30 minutes, a solution of the above alcohol (6.0 g, 27.0 mmol) in dichloromethane was added. After the addition was complete, the reacting mixture was stirred at −78° C. for half an hour, and treated with triethylamine (18.8 mL, 135 mmol). The resultant mixture was stirred at −78° C. for ˜30 minutes, 0° C. for 2 hours. The product mixture was quenched with saturated aqueous ammonium chloride. The organic layer was washed with brine, dried over sodium sulfate, filtered and concentrated under vacuum. The residue was subjected to column chromatography on silica gel eluting with a 0% to 60% ethyl acetate/hexane gradient. Collection and concentration of appropriate fractions afforded title compound, 6-(benzyloxy)oxepan-3-one. ¹H NMR (400 MHz, CDCl₃): δ 7.33 (m, 5H); 4.64-4.52 (m, 2H); 4.16-4.09 (m); 4.07-3.98 (m); 3.84-3.77 (m); 3.69 (m); 3.01-2.92 (m, 1H); 2.57-2.49 (m); 1.98-1.93 (m, 2H).

### Step 3: N-5-{[(4-Fluorolbenzyl)amino]carbonyl}-4-hydroxy-3-oxo-10-oxa-2,6-diazatricyclo[6.3.2.0^{2,7}]trideca-4,6-dien-8-yl)-N,N′,N′-trimethylethanediamide

Following the procedures described in Example 1, Steps 1 to 9, and substituting 4-benzyloxymethylcyclohexanone with 6-(benzyloxy)oxepan-3-one in Step 1 of Example 1, the title compound was prepared as a racemic mixture. Chiral column chromatography separation of the material provided a faster eluting enantiomer (Compound 23A) and a slower eluting enantiomer (Compound 23B).

Compound 23A: ¹H NMR of Example 23A (400 MHz, CDCl₃): δ 9.78 (br s, 1 H); 7.37 (dd, J=8.3, 5.4 Hz, 2H); 6.98 (t, J=8.6 Hz, 2H); 5.37 (t, J=5.14 Hz, 1H); 4.54 (d, J=6.4 Hz, 2H); 4.13-3.98 (m, 2H); 3.63 (d, J=12.7 Hz, 1H); 3.03 (s, 3H); 2.98 (s, 3H); 2.91 (s, 3H); 2.65 (td, J=11.7, 5.4 Hz, 1H); 2.36 (br t, 12 Hz, 1H); 2.00-1.89 (m, 1H). HR MS: ESI=488.1937 (M+1); calculated 488.1945 (M+1).

Compound 23B: HR MS: ESI=488.1940 (M+1); calculated 488.1945 (M+1).

### Example 24

Enantiomers of N-5-{[(4-fluorolbenzyl)amino]carbonyl}-4-hydroxy-3-oxo-2,6-diazatricyclo[6.3.2.0^{2,7}]trideca-4,6-dien-8-yl)-N,N′,N′-trimethylethanediamide

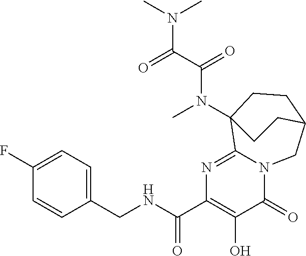

Following the procedures described in Example 1, Steps 1 to 9, and substituting 4-benzyloxymethylcyclohexanone with 4-(benzyloxy)cycloheptanone (Angew. Chem., Int. Ed., 2002, 3031-3033) in Step 1 of Example 1, the title compound was prepared as a racemic mixture. Chiral column chromatography separation of the material provided a faster eluting enantiomer (Compound 24A) and a slower eluting enantiomer (Compound 2413).

Compound 24A: ¹H NMR (400 MHz, CDCl₃): δ 9.53 (br s, 1H); 7.36 (dd, J=8.3, 5.4 Hz, 1H); 6.98 (t, J=8.5 Hz, 1H); 4.54 (m, 1H); 3.02 (s, 3H); 3.01 (s, 3H); 2.97 (s, 3H); 2.29-1.83 (m). HR MS: ESI=486.2157 (M+1); calculated 486.2153 (M+1).

Compound 24B: HR MS: ESI=486.2153 (M+1); calculated 486.2153 (M+1).

### Example 25

### Isomers of N-(8-ethyl-4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N,N′,N′-trimethylethanediamide

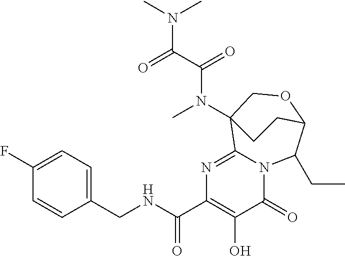

### Step 1: tert-Butyl[1-(3,4-dihydro-2H-pyran-2-yl)propoxy]dimethylsilane

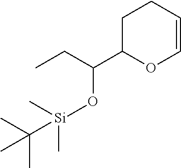

To a cold (−30° C.) solution of 3,4-dihydro-2H-pyran-2-carbaldehyde (34.8 mL, 335 mmol) in anhydrous ether (1 L) under a nitrogen atmosphere, an ether solution of ethylmagnesium bromide (112 mL, 335 mmol, 3M) was added drop wise over 20 minutes. The resulting mixture was slowly warmed to room temperature over 4 hours, quenched with water (500 mL) and kept basic with 1N NaOH (50 mL.) The product was extracted into ether (4×350 mL). The combined organic extract was washed successively with water and brine, dried over anhydrous sodium sulfate, filtered, and concentrated under reduced pressure. The residue was then redissolved in dry DMF (200 mL) under a nitrogen atmosphere and cooled in an ice bath. Imidazole (23 g, 335 mmol) and tert-butyl dimethylsilyl chloride (51 g, 335 mmol) were added and stirred at room temperature overnight. Additional imidazole (38 g, 558 mmol) and tert-butyl dimethyl-silyl chloride (51 g, 335 mmol) were then added and stirred at room temperature overnight for reaction to go to completion. The reaction was concentrated under reduced pressure and the residue was redissolved in ether (1 L). The ether solution was washed successively with water and brine solution, dried over anhydrous sodium sulfate, filtered, and concentrated under reduced pressure. The residue was redissolved in hexanes and filtered through a silica gel plug eluting with hexanes (1500 mL). Concentrated of the eluent afford the title compound. ¹H NMR (400 MHz, CDCl₃): δ: 6.35 (m, 1H), 4.67 (m, 1H), 3.8-3.6 (m, 2H), 2.12-1.80 (m, 3H), 1.75-1.32 (m, 3H), 0.90 (br s, 12H), 0.07 (br s, 6H).

### Step 2: 6-(1-{[tert-Butyl(dimethyl)silyl]oxy}) propyl)dihydro-2H-pyran-3(4H)-one

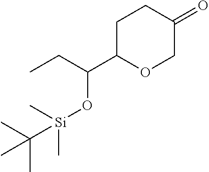

To a cold (0° C.) solution of tert-butyl[1-(3,4-dihydro-2H-pyran-2-yl)propoxy]dimethylsilane (58 g, 227 mmol) in dry THF (1 L) under a nitrogen atmosphere, a solution of 9-BBN in THF (455 mL, 227 mmol, 0.5 M) was added drop wise over 40 minutes. The reaction was allowed to warm to room temperature over 18 hours. A suspension of sodium perborate tetrahydrate (105 g, 682 mmol) in water (300 mL) was added slowly and the resulting mixture was stirred for 3 hours. The product was extracted into ether (3×300 mL). The combined organic layer was washed successively with water and brine solution, dried over anhydrous sodium sulfate, filtered, and concentrated under reduced pressure. Purification of the residue by flash column chromatography on silica gel (1.5 kg) using a gradient elution of 0-40% ethyl acetate in hexane provided the intermediate alcohol. To a stirred solution of the alcohol (29 g, 106 mmol) in dichloromethane (600 mL) was added sodium acetate (3.0 g, 37 mmol) and pyridinium chlorochromate (40 g, 186 mmol) and the mixture was stirred for 18 hours at room temperature. The reaction was quenched with isopropyl alcohol (5 mL), diluted with ether (700 mL) and filtered through a plug of Fluorosil. The filtrate was concentrated under reduced pressure and the residue was purified by flash column chromatography on silica gel (330 g) using a gradient elution of 0-40% ethyl acetate in hexane gradient to give the desired title product. ¹H NMR (400 MHz, CDCl₃): δ: 4.15 (m, 1H), 3.92 (m, 1H), 3.75-3.52 (m, 2H), 2.62 (m, 1H), 2.44 (m, 1H), 2.10-1.90 (m, 2H), 1.70-1.34 (m, 2H), 0.90 (br s, 12H), 0.07 (br s, 6H).

### Step 3: tert-Butyl[6-(1-{tert-butyl(dimethyl)silyloxy}propyl)-3-cyanotetrahydro-2H-pyran-3-yl]methylcarbamate

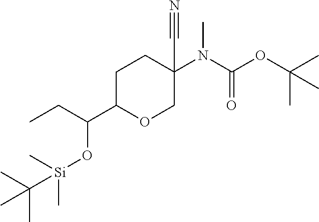

A solution of 6-(1-{[tert-butyl(dimethyl)silyl]oxy}propyl)-dihydro-2H-pyran-3(4H)-one (6.5 g, 24 mmol), methylamine hydrochloride (1.8 g, 27 mmol), and sodium cyanide (1.3 g, 27 mmol) in 4:1 methanol:water (65 mL) was stirred for 24 hours at room temperature. The solution was made basic (pH=9) with a saturated aqueous solution of sodium bicarbonate and the product was extracted into ethyl acetate (3×200 mL). The combined organic layer was washed with brine solution, dried over anhydrous sodium sulfate, filtered, and concentrated under reduced pressure. The residue was dissolved in dichloromethane (70 mL) and treated with di-tert-butyl dicarbonate (8.3 g, 38 mmol), triethylamine (5.4 mL, 39 mmol) and DMAP (100 mg, 0.82 mmol). The solution was stirred at room temperature overnight, additional di-tert-butyl dicarbonate (2 g, 9.2 mmol) and triethylamine (1 mL, 7.2 mmol) were added, and the solution was aged another 60 hours at room temperature. The product mixture was concentrated under reduced pressure, and the residue was purified by flash column chromatography on silica gel (120 g) using a gradient elution of 0-40% ethyl acetate in hexane to give the desired product: ES MS: m/z=413.2 (M+1).

### Step 4: tert-Butyl[6-(1-{[tert-butyl(dimethyl)silyl]oxy}propyl)-3-(N′-hydroxycarbamimidoyl)tetrahydro-2H-pyran-3-yl]methylcarbamate

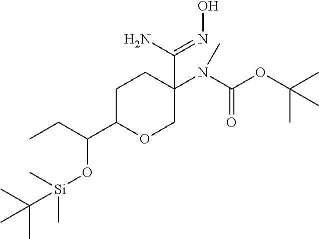

To a solution of tert-butyl[6-(1-{[tert-butyl(dimethyl)silyl]oxy}propyl)-3-cyanotetrahydro-2H-pyran-3-yl]methylcarbamate (3.7 g, 8.9 mmol) in methanol (50 mL) was added a 50% aqueous solution of hydroxylamine (0.95 mL, 15.5 mmol), and the mixture was stirred at 60° C. for 24 hours. The solution was concentrated under reduced pressure. The residue was redissolved in methanol and concentrated under reduced pressure (2×50 mL) to remove traces of hydroxylamine and water. The crude product was used without purification in the next step: ES MS: m/z=446.2 (M+1).

### Step 5: Dimethyl (2E/Z)-2-({[(E/Z)-amino{3-[(tert-butoxycarbonyl)(methyl)-amino]-6-(1-{tert-butyl(dimethyl)silyl]oxy}propyl)tetrahydro-2H-pyran-3-yl}methylidene]amino}oxy)but-2-enedioate

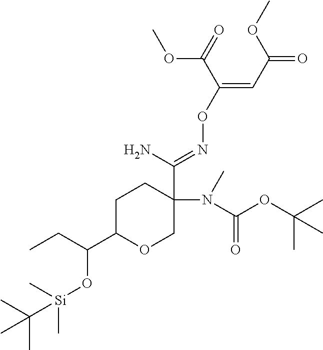

To a stirred solution of tert-butyl[6-(1-{[tert-butyl(dimethyl)silyl]oxy}-propyl)-3-(N′-hydroxycarbamimidoyl)tetrahydro-2H-pyran-3-yl]methylcarbamate (4.0 g, 8.9 mmol) in methanol (50 mL) under nitrogen at 0° C. was added dimethyl acetylenedicarboxylate (1.3 mL, 10.2 mmol). The reaction was stirred at 0° C. for 2 hours and then allowed to warm to room temperature with stirring for 18 hours. The solvent was removed under reduced pressure, and the residue was purified by flash column chromatography on silica gel (80 g) using a gradient elution of 0-50% ethyl acetate in hexane gradient to give the desired product: ES MS: m/z=588.1 (M+1).

### Step 6: Methyl 2-({3-[(tert-butoxycarbonyl)(methyl)amino]-6-(1-{[tert-butyl(dimethyl)silyl]oxy}propyl)tetrahydro-2H-pyran-3-yl}-5,6-dihydroxypyrimidine-4-carboxylate

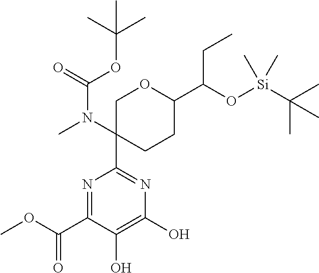

A solution of dimethyl (2E/Z)-2-({[(E/Z)-amino{3-[(tert-butoxycarbonyl)-(methyl)amino]-6-(1-{[tert-butyl(dimethyl)silyl]oxy}propyl)tetrahydro-2H-pyran-3-yl}methylidene]amino}oxy)but-2-enedioate (3.1 g, 5.3 mmol) and DIEA (0.93 mL, 5.3 mmol) in o-xylene (160 mL) was heated at 135° C. for 24 hours under a nitrogen atmosphere. The solution was cooled, water and 1N HCl (6 mL) were added, and the product was extracted into EtOAc (3×300 mL). The combined organic layer was dried over anhydrous sodium sulfate, filtered, and concentrated under reduced pressure. The residue was stirred in ether (100 mL) and the undissolved impurities were filtered away. The filtrate was concentrated under reduced pressure and the product was used in the next step without further purification: ES MS: m/z=556.2 (M+1).

### Step 7: tert-Butyl[6-(1-{[tert-butyl(dimethyl)silyl]oxy}propyl)-3-{4-[(4-fluorobenzyl)carbamoyl]-5,6-dihydroxypyrimidin-2-yl}tetrahydro-2H-pyran-3-yl]methylcarbamate

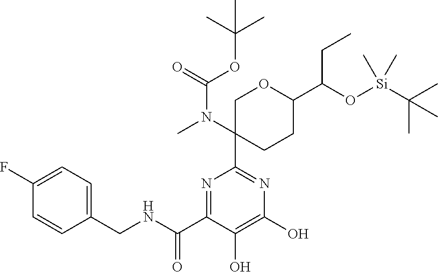

A solution of methyl 2-{3-[(tert-butoxycarbonyl)(methyl)amino]-6-(1-{[tert-butyl(dimethyl)silyl]oxy}propyl)tetrahydro-2H-pyran-3-yl}-5,6-dihydroxypyrimidine-4-carboxylate (3.0 g, 5.3 mmol) and 4-fluorobenzylamine (2.45 mL, 21 mmol) in isopropanol (60 mL) was heated at 60° C. for 10 hours. The solution was cooled, diluted with ethyl acetate (250 mL), and washed with aqueous hydrochloric acid (40 mL of a 0.5 M solution). The organic layer was separated, washed successively with water and brine, dried over anhydrous sodium sulfate, filtered, and the solvent was removed under reduced pressure. The residue was purified by flash column chromatography on silica gel (40 g) using a gradient elution of 0-100% ethyl acetate in hexane to give the desired product. ES MS: m/z=649.2 (M+1)

### Step 8: tert-Butyl[3-{4-[(4-fluorobenzyl)carbamoyl]-5,6-dihydroxypyrimidin-2-yl}-6-(1-hydroxypropyl)tetrahydro-2H-pyran-3-yl]methylcarbamate

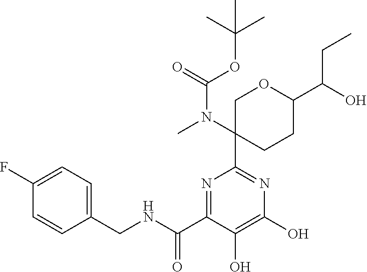

To a solution of tert-butyl[6-(1-{[tert-butyl(dimethyl)silyl]oxy}propyl)-3-{4-[(4-fluorobenzyl)carbamoyl]-5,6-dihydroxypyrimidin-2-yl}tetrahydro-2H-pyran-3-yl]methylcarbamate (2.7 g, 4.16 mmol) dissolved in acetonitrile (50 mL) in a Teflon vial, was added aqueous HF (48 wt. % solution, 0.75 mL, 21 mmol) and the resulting mixture was stirred overnight at room temperature. The reaction mixture was then diluted with water and aqueous sodium bicarbonate to raise the pH of the solution to 3. The product was extracted into EtOAc (3×75 mL) and the combined organic layer was washed with brine solution, dried over anhydrous sodium sulfate, filtered, and concentrated under reduced pressure. The crude product was azeotroped once with toluene and dried under vacuum overnight.: ES MS: m/z=535.2 (M+1).

### Step 9: tert-Butyl (8-ethyl-4-{[(4-fluorobenzyl)amino]carbonyl}-5-hydroxy-6-oxo-10-oxa-3,7-diazatricyclo[7.2.2.0^{2,7}]trideca-2,4-dien-1-yl)methyl-carbamate

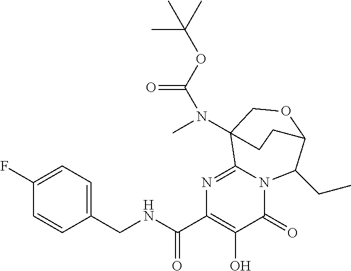

To a cold (0° C.) solution of tert-butyl[3-{4-[(4-fluorobenzyl)carbamoyl]-5,6-dihydroxypyrimidin-2-yl}-6-(1-hydroxypropyl)tetrahydro-2H-pyran-3-yl]methyl-carbamate (2.1 g, 3.93 mmol) and triethylamine (3.0 mL, 21.6 mmol) in dry acetonitrile (50 mL) under nitrogen, methanesulfonyl chloride (1.45 mL, 18.6 mmol) was added. The mixture was stirred for 4 hours at 0° C. and then concentrated under reduced pressure. The residue was redissolved in EtOAc (150 mL) and successively washed with dilute aqueous HCl (40 mL of a 0.5M solution), dilute sodium bicarbonate (40 mL), and brine. The organic layer was dried over anhydrous sodium sulfate, filtered, and concentrated under reduced pressure. The crude trismesylate was used without further purification. ES MS: m/z=769.1 (M+1).

Potassium carbonate (1.45 g, 10.5 mmol) was added to a stirred solution of the trismesylate (2.3 g, 3.0 mmol) in dry dimethylacetamide (150 mL) under a nitrogen atmosphere. The reaction mixture was placed in an oil bath preheated to 120° C. and stirred for 80 minutes. The solution was cooled, diluted with dilute aqueous HCl (100 mL of a 0.2M solution) and extracted into ethyl acetate (2×250 mL). The combined organic layer was dried over anhydrous sodium sulfate, filtered, and concentrated under reduced pressure. The residue was stirred in ether (250 mL) and the undissolved impurities were filtered away. The filtrate was concentrated under reduced pressure and the product was used in the next step without further purification.

ES MS: m/z=517.1 (M+1).

### Step 10: 6-Ethyl-N-(4-fluorobenzyl)-5-hydroxy-1-(methylamino)-6-oxo-10-oxa-3,7-diazatricyclo[7.2.2.0^{2,7}]trideca-2,4-diene-4-carboxamide hydrochloride

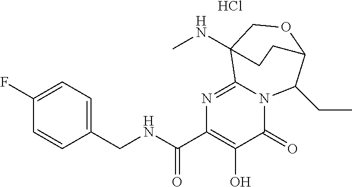

tert-Butyl (8-ethyl-4-{[(4-fluorobenzyl)amino]carbonyl}-5-hydroxy-6-oxo-10-oxa-3,7-diazatricyclo[7.2.2.0^{2,7}]trideca-2,4-dien-1-yl)methylcarbamate (1.85 g, 3.6 mmol) was dissolved in HCl/dioxane (30 mL of a 4 M solution) and stirred for 3 hours. The solution was concentrated under reduced pressure. The residue was dissolved in methanol (2×40 mL) and concentrated under reduced pressure. The crude product was redissolved in a minimal amount of methanol and diluted with water. The methanol was removed under reduced pressure to precipitate a solid in the remaining water. The solid was filtered away and the filtrate was concentrated under reduced pressure, azeotroped with CH₃CN (2×20 mL), and dried under high vacuum. The crude product was used without purification in the next step: ES MS: m/z=417.1 (M+1)

### Step 11: N-(8-Ethyl-4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N,N′,N′-trimethylethanediamide

To a stirred solution of 6-ethyl-N-(4-fluorobenzyl)-5-hydroxy-1-(methylamino)-6-oxo-10-oxa-3,7-diazatricyclo[7.2.2.0^{2,7}]trideca-2,4-diene-4-carboxamide hydrochloride (1030 mg, 2.27 mmol) in dry dichloromethane (40 mL) under nitrogen was added NMM (1.0 mL, 9.1 mmol), N,N-dimethyloxamic acid (373 mg, 3.18 mmol), HOAt (371 mg, 2.73 mmol), and EDC (523 mg, 2.73 mmol). The reaction was refluxed for 6 hours. To the reaction mixture was added more NMM, HOAT, EDC and N,N-dimethyloxamic acid in the amounts previously added. The reaction mixture was refluxed another 18 hours. After cooling, the reaction mixture was diluted with aqueous hydrochloric acid (50 mL, 0.5 M) and extracted into dichloromethane (3×60 mL). The combined organic layer was dried over anhydrous sodium sulfate, filtered, and concentrated under reduced pressure. The crude product was purified by reverse phase HPLC (C18 column) using a water:acetonitrile containing 0.1% TFA mobile phase gradient (25-60% acetonitrile over minutes, 85 mL/minute). Lyophilization of product containing fractions gave the desired product as an amorphous white solid. This mixture of four diastereomers were then separated by chiral chromatography (stationary phase=Chiralcel OD/OJ; isocratic elution) and the fractions concentrated under reduced pressure: the first and fourth eluting diastereomers (Compound 25A and Compound 25D respectively) were separated away from the second and third (Compound 25B and Compound 25D respectively) on a Chiralcel OD column with 100% ethanol containing 0.1% TFA. Fractions for the second and third eluting diastereomers were combined, concentrated under reduced pressure, and then separated on a Chiralcel OJ column using 60% ethanol/heptane containing 0.1% TFA.

Compound 25A (first eluting diastereomer: ether linkage and ethyl side-chain anti to one another (enantiomer A)): ¹H NMR (400 MHz, DMSO): δ 9.7 (br. s, 1H); 7.36 (dd, J=8.4, 5.5 Hz, 2H); 6.97 (t, J=8.7 Hz, 2H); 4.95 (m, 1H); 4.55 (m, 3H); 4.13 (d, J=9.3 Hz, 1H); 4.00 (d, J=9.2 Hz, 1H); 3.40 (m, 1H); 3.04 (s, 3H); 2.99 (s, 3H); 2.89 (s, 3H); 2.24 (m, 2H); 2.13 (m, 2H); 1.56 (m, 1H); 1.04 (t, J=7.4 Hz, 3H). HR MS: ESI=516.2269 (M+1); calculated: 516.2253 (M+1).

Compound 25B (second eluting diastereomer: ether linkage and ethyl side-chain anti to one another (enantiomer B)): ¹H NMR (399 MHz, DMSO): δ 12.3 (s, 1H); 9.7 (br. s, 1 H); 7.37 (dd, J=8.4, 5.5 Hz, 2H); 6.97 (t, J=8.7 Hz, 2H); 4.94 (m, 1H); 4.55 (m, 3H); 4.12 (d, J=9.5 Hz, 1H); 3.99 (d, J=9.2 Hz, 1H); 3.40 (m, 1H); 3.04 (s, 3H); 2.99 (s, 3H); 2.88 (s, 3H); 2.23 (m, 2H); 2.12 (m, 2H); 1.56 (m, 1H); 1.04 (t, J=7.5 Hz, 3H). HR MS: ESI=516.2263 (M+1); calculated: 516.2253 (M+1).

Compound 25C (third eluting diastereomer: ether linkage and ethyl side-chain syn to one another (enantiomer A)): ¹H NMR (399 MHz, DMSO): δ 12.1 (br. s, 1H); 9.66 (br. s, 1H); 7.37 (dd, J=8.2, 5.5 Hz, 2H); 6.98 (t, J=8.6 Hz, 2H); 5.29 (d, J=11.9 Hz, 1H); 4.60 (m, 2H); 4.48 (m, 2H); 3.91 (d, J=11.9 Hz, 1H); 3.01 (m, 6H); 2.98 (s, 3H); 2.45 (m, 1H); 2.18 (m, 2H); 1.94 (m, 2H); 1.46 (m, 1H); 1.14 (t, J=7.3 Hz, 3H). HR MS: ESI=516.2271 (M+1); calculated: 516.2253 (M+1).

Compound 25D (fourth eluting diastereomer: ether linkage and ethyl side-chain syn to one another (enantiomer B)). ¹H NMR (399 MHz, DMSO): δ 9.66 (br. s, 1H); 7.35 (dd, J=8.6, 5.3 Hz, 2H); 6.98 (t, J=8.7 Hz, 2H); 5.29 (d, J=12.0 Hz, 1H); 4.60 (m, 2H); 4.48 (m, 2H); 3.92 (d, J=12.0 Hz, 1H); 3.02 (m, 6H); 2.98 (s, 3H); 2.45 (m, 1H); 2.18 (m, 2 H); 1.94 (m, 2H); 1.46 (m, 1H); 1.14 (t, J=7.3 Hz, 3H). HR MS: ESI=516.2273 (M+1); calculated: 516.2253 (M+1).

### Example 26

Isomers of N′-(8-ethyl-4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N-dimethylethanediamide

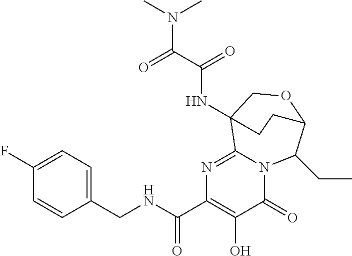

The title compound was synthesized using the procedures given in Example 25 except that ammonium chloride was used in place of methylamine hydrochloride in Step 3. ES MS 502.2. The four isomeric compounds were separated by chiral chromatography:

Compound 26A & enantiomer 26B (ether linkage and ethyl side-chain anti to one another).

Compound 26C & enantiomer 26D (ether linkage and ethyl side-chain syn to one another (enantiomer A)).

### Example 27

Isomers of N-(4-{[(4-fluorobenzyl)amino]carbonyl}-5-hydroxy-8-methyl-6-oxo-10-oxa-3,7-diazatricyclo[7.2.2.0^{2,7}]trideca-2,4-dien-1-yl)-N,N′,N′-trimethylethanediamide

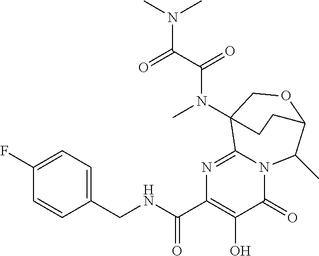

The title compound was synthesized using the procedures given in Example 25 except that methyl magnesium bromide was used in place of ethyl magnesium bromide in Step 1. ES MS 502.2. The four isomeric final products were separated by chiral chromatography.

Compound 27A & enantiomer 27B (ether linkage and methyl side-chain syn to one another (enantiomer A)).

Compound 27C & enantiomer 27D (ether linkage and methyl side-chain anti to one another (enantiomer A)).

### Example 28

Isomers of N-(4-{[(4-fluorobenzyl)amino]carbonyl}-5-hydroxy-8-methyl-6-oxo-10-oxa-3,7-diazatricyclo[7.2.2.0^{2,7}]trideca-2,4-dien-1-yl)-N,N′-dimethylethanediamide

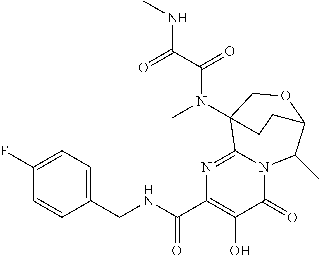

The title compound was synthesized using the procedures given in Example 25 except that methyl magnesium bromide was used in place of ethyl magnesium bromide in Step 1 and N-methyloxamic acid was used in place of N,N-dimethyloxamic acid in Step 11. ES MS 488.2. The mixture of isomers was separated into two sets of enantiomers with C18 reverse phase HPLC.

Compound 28A (first eluting pair of enantiomers—ether linkage and methyl side-chain syn to one another).

Compound 28B (second eluting pair of enantiomers—ether linkage and methyl side-chain anti to one another).

### Example 29

### N′-(4-{[(4-Fluorobenzyl)amino]carbonyl}-5-hydroxy-8-methyl-6-oxo-10-oxa-3,7-diazatricyclo[7.2.2.0^{2,7}]trideca-2,4-dien-1-yl)-N-dimethylethanediamide

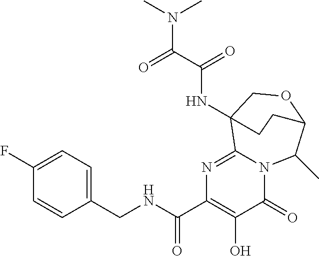

The title compound was synthesized using the procedure given in Example 25 except that methyl magnesium bromide was used in place of ethyl magnesium bromide in Step 1 and ammonium chloride was used in place of methylamine hydrochloride in Step 3. ES MS 488.2 (M+1). The four isomeric final products were separated by chromatography on ChiralPak AD eluting with 60% ethanol in hexane (0.5 of trifluoroacetic acid as modifier)

Compound 29A (first eluting diastereomer: ether linkage and methyl side-chain syn to one another (enantiomer A)).

Compound 29B (second eluting diastereomer: ether linkage and methyl side-chain syn to one another (enantiomer B)).

Compound 29C (third eluting diastereomer: ether linkage and methyl side-chain anti to one another (enantiomer A)).

Compound 29D (fourth eluting diastereomer: ether linkage and methyl side-chain anti to one another (enantiomer B)).

### Example 30

### N-5-{[(4-Fluorolbenzyl)amino]carbonyl}-4-hydroxy-3-oxo-2,6-diazatricyclo[6.2.2.02]dodeca-4,6-dien-8-yl)-N,N′,N′-trimethylethanediamide

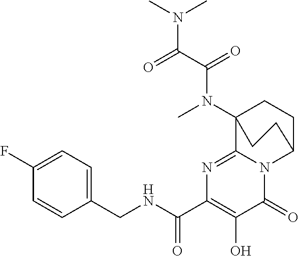

### Step 1: tert-Butyl[4-(benzyloxy)-cyancyanocyclohexyl]methylcarbamate

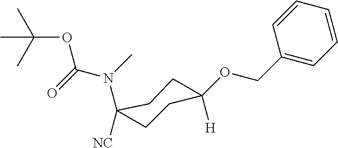

To a mixture of 4-(benzyloxy)cyclohexanone (14.3 g, 70.2 mmol) (synthesized in accordance with the procedure in US 2006292073, pages 11-12) methylamine hydrochloride (19.0 g, 280 mmol), and sodium cyanide (17.8 g, 280 mmol) in a 1:1 mixture of dioxane:water (150 mL) was stirred at room temperature for 48 hours. The reaction mixture was concentrated under vacuum. The residue was partitioned between ethyl acetate and water. The organic extract was washed with brine, dried over anhydrous sodium sulfate, filtered, and concentrated under vacuum to afford the intermediate 4-(benzyloxy)-1-(methylamino)cyclohexanecarbonitrile as a mixture of cis and trans isomers. A solution of the aminocyclohexanecarbonitrile (16.8 g, 68.8 mmol) and di-tert-butyl dicarbonate (45 g, 206 mmol) in dioxane (250 mL) was heated at 40° C. for 6 days. The product mixture was concentrated under vacuum, and the residue partitioned between ethyl acetate and water. The organic extract was dried over sodium sulfate, filtered, and concentrated under vacuum. The ˜1:1 mixture of faster and slower eluting diastereoisomers was separated by column chromatography on silica gel eluting with a 0% to 50% ethyl acetate/hexane gradient. Collection and concentration of faster eluting isomer afford white solid. Collection and concentration of slower eluting fractions afforded colorless oil. ¹H NMR of faster eluting isomer (400 MHz, CDCl₃): δ 7.37-7.33 (m, 5H); 4.50 (s, 2H); 3.68 (br signal, 1H); 2.92 (s, 3 H); 2.22-2.01 (m, 8H); 1.51 (s, 9H). ¹H NMR of slower eluting isomer (400 MHz, CDCl₃): δ 7.38-7.32 (m, 5H); 4.56 (s, 2H); 3.36 (m, 1H); 2.92 (s, 3H); 2.75-1.73 (sets of multiplets); 1.52 (s, 9H). Both isomers were carried through the following reaction sequence. Precursor derived from the faster eluting isomer underwent base induced cyclization to form the diazatricyclododecane core, while the corresponding intermediate derived from the slower eluting isomer did not.

### Step 2: N-5-{[(4-Fluorobenzyl)amino]carbonyl}-4-hydroxy-3-oxo-2,6-diazatricyclo[6.2.2.0^{2,7}]dodeca-4,6-dien-8-yl)-N,N′,N′-trimethylethanediamide

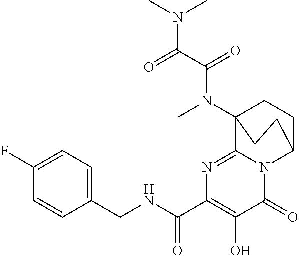

Following the procedures described in Example 1, Steps 2 to 9, the title compound was prepared wherein in Step 2, tert-butyl{trans-4-[(benzyloxy)-methyl]-1-cyanocyclohexyl}methylcarbamate was substituted with tert-butyl[4-(benzyloxy)-1-cyanocyclo-hexyl]methylcarbamate (faster eluting diastereoisomer), and in Step 5 the hydrogenolysis was carried out in methanol in the presence of Pearlman catalyst under ˜45 psi of hydrogen for 5 days at room temperature. ¹H NMR (400 MHz, DMSO): δ 9.61 (t, J=6.52 Hz, 1H); 7.37 (dd, J=8.31, 5.56 Hz, 2H); 7.16 (t, J=8.79 Hz, 2H); 5.07 (br s, 1H); 4.47 (d, J=6.52 Hz, 2H); 2.96 (s, 3H); 2.90 (s, 3H); 2.88 (s, 3H); 2.09-1.94 (m, 4H); 1.73 (m, 2H). HR MS: ESI=472.2012 (M+1); calculated 472.1996 (M+1).

### Example 31

### HIV Integrase Assay: Strand Transfer Catalyzed by Recombinant Integrase

Assays for the strand transfer activity of HIV-1 integrase were conducted in accordance with WO 02/30930 for recombinant integrase. Representative compounds of the present invention exhibit inhibition of strand transfer activity in this assay. For example, the compounds prepared in Examples 1 to 6, Examples 10A to 13B, and Example 24B were tested in the integrase assay and found to have the IC₅₀ values in Table B.

TABLE B

| Compound | IC₅₀ (nM) |
|---|---|
| 1 | 13 |
| 2 | 15 |
| 3 | 32 |
| 4 | 14 |
| 5 | 33 |
| 6 | 37 |
| 10A | 5 |
| 10B | 14 |
| 11A | 37 |
| 11B | 14 |
| 12A | 11 |
| 12B | 18 |
| 13A | 13 |
| 13B | 13 |
| 24B | 24 |

Further description on conducting the assay using preassembled complexes is found in Wolfe, A. L. et al., J. Virol. 1996, 70: 1424-1432, Hazuda et al., J. Virol. 1997, 71: 7005-7011; Hazuda et al., Drug Design and Discovery 1997, 15: 17-24; and Hazuda et al., Science 2000, 287: 646-650.

### Example 32

### Assay for Inhibition of HIV Replication

Assays for the inhibition of acute HIV-1 infection of T-lymphoid cells were conducted in accordance with Vacca, J. P. et al., Proc. Natl. Acad. Sci. USA 1994, 91: 4096. Representative compounds of the present invention exhibit inhibition of HIV replication in this assay (also referred to herein as the “spread assay”). For example, except for Compound 29B, the compounds of Examples 1 to 30 were tested in this assay and all were found to have IC₉₅ values of less than about 50 nM. The specific values for the compounds are shown in Table C.

(Note: Compound 29B was not tested in the spread assay.)

TABLE C

| | IC₉₅ (nM) |
|---|---|
| | in the presence of |
| Compound | 10% FBS |
| 1 | 6.2 |
| 2 | 7.2 |
| 3 | 11 |
| 4 | 39 |
| 5 | <3.9 |
| 6 | 5.3 |
| 7 | 9.6 |
| 8 | 9.2 |
| 9 | 15. |
| 10A | 9.0 |
| 10B | 15 |
| 11A | 17 |
| 11B | 7.8 |
| 12A | 11 |
| 12B | 19 |
| 13A | 8.6 |
| 13B | 20 |
| 14 | 13.1 |
| 15 | 9.5 |
| 16 | 11.3 |
| 17 | 20.5 |
| 18A | 9.8 |
| 18B | 10.7 |
| 19 | 15.9 |
| 20A | 11.0 |
| 20B | 16.7 |
| 21A | 20.0 |
| 21B | 21.0 |
| 22A | 12.7 |
| 22B | 12.6 |
| 23A | 16.7 |
| 23B | 23.6 |
| 24A | 11.7 |
| 24B | 7.4 |
| 25A | 6.3 |
| 25B | 7.9 |
| 25C | 10.8 |
| 25D | 7.2 |
| 26A | 16.0 |
| 26B | 22.6 |
| 26C | 18.1 |
| 26D | 5.1 |
| 27A | 7.5 |
| 27B | 27.3 |
| 27C | 7.7 |
| 27D | 6.9 |
| 28A | 17.8 |
| 28B | 15.9 |
| 29A | 17.9 |
| 29C | 47.8 |
| 29D | 21.6 |
| 30 | 22.4 |

### Example 33

### Assay for Inhibition of HIV Integrase Mutant Virus Replication

An assay for measuring the inhibition of acute HIV-1 infection with HeLa P4-2 cells in a single cycle infectivity assay was conducted using methods described in Joyce et al., J. Biol. Chem. 2002, 277: 45811, Hazuda et al., Science 2000, 287: 646, and Kimpton et al, J. Virol. 1992, 66: 2232. Proviral plasmids encoding viruses containing specific mutations in the integrase gene (N155H, Q148R, Y143R, E92Q, or G140S/Q148H) were generated by site-directed mutagenesis, and viruses were produced by transfecting 293T cells with the appropriate proviral plasmids. Representative compounds of the present invention exhibit inhibition of HIV replication in the mutant assays For example, the compounds of Examples 1 to 28A and 29 to 30 were found to have the IC₅₀ values in these assays shown in Table D. (Note: Example 28B was not tested in this assay.)

TABLE D

| | | N155H | Q148R | Y143R | G140S/Q148H |
|---|---|---|---|---|---|
| Example No. | IC₅₀ (nM) | (shift)¹ | (shift)¹ | (shift)¹ | (shift)¹ |
| 1 | 7.2 | 3 | 2 | 2 | 15 |
| 2 | 4.0 | 2 | 2 | 1 | 5 |
| 3 | 31 | 2 | 2 | 1 | 9 |
| 4 | 33 | 4 | 3 | 1 | 4 |
| 5 | 6.7 | 2 | 1 | 1 | 9 |
| 6 | 9.0 | 2 | 2 | 1 | 5 |
| 7 | 80 | 3 | 4 | 2 | 44 |
| 8 | 13 | 16 | 18 | 13 | 110 |
| 9 | 43 | 5 | 11 | 2 | 40 |
| 10A | 221 | 5 | 4 | 1 | 26 |
| 10B | 30 | 18 | 22 | 6 | 12 |
| 11A | 21 | 10 | 8 | 2 | >79 |
| 11B | 112 | 5 | 2 | 1 | 14 |
| 12A | 23 | 6 | 7 | 2 | 112 |
| 12B | 7.0 | 56 | 32 | 7 | 240 |
| 13A | 7.0 | 2 | 1 | 2 | 2 |
| 13B | 8.0 | 30 | 57 | 7 | >209 |
| 14 | 12 | 2 | 1 | 2 | 4 |
| 15 | 23 | 2 | 1 | 1 | 9 |
| 16 | 15 | 3 | 1 | 1 | 15 |
| 17 | 46 | 3 | 3 | 1 | 38 |
| 18A | 19 | 8 | 7 | 2 | >88 |
| 18B | 10 | 2 | 2 | 2 | 19 |
| 19 | 8 | 45 | 59 | 7 | >209 |
| 20A | 8 | 3 | 2 | 1 | 21 |
| 20B | 17 | 7 | 11 | 1 | 102 |
| 21A | 20 | 3 | 3 | 1 | 73 |
| 21B | 18 | 13 | 11 | 3 | >93 |
| 22A | 7 | 23 | 24 | 5 | 198 |
| 22B | 19 | 3 | 3 | 1 | 10 |
| 23A | 21 | 7 | 6 | 1 | 84 |
| 23B | 5 | 136 | 253 | 7 | 229 |
| 24A | 15 | 4 | 5 | 1 | >111 |
| 24B | 7 | 3 | 1 | 2 | 6 |
| 25A | 14 | 1 | 1 | 7 | 1 |
| 25B | 8 | 1 | 1 | 1 | 1 |
| 25C | 6 | 6 | 4 | 3 | 88 |
| 25D | 15 | 98 | 38 | 3 | >111 |
| 26A | 24 | 9 | 9 | 2 | >70 |
| 26B | 36 | 1 | 1 | 1 | 1 |
| 26C | 23 | 12 | 9 | 2 | 26 |
| 26D | 16 | 1 | 1 | 2 | 1 |
| 27A | 20 | 1 | 1 | 1 | 2 |
| 27B | 24 | 20 | 9 | 7 | >70 |
| 27C | 15 | 11 | 12 | 3 | >111 |
| 27D | 11 | 1 | 1 | 1 | 2 |
| 28A | 46 | 3 | 1 | 1 | 4 |
| 29A | 20 | 1 | 1 | 1 | 4 |
| 29B | 69 | 1 | 3 | 2 | 2 |
| 29C | 20 | 17 | 8 | 3 | 81 |
| 29D | 21 | 2 | 1 | 1 | 2 |
| 30 | 17 | 7 | 9 | 3 | 32 |
| Compound X² | 52 | 13 | 22 | 15 | 400 |
| Compound Y³ | 16 | 15 | 26 | 1 | 410 |
| Compound Z⁴ | 34 | 32 | >34 | 1 | >34 |

¹“Shift” means the number of fold shift in IC₅₀ versus wild type IIIB. A number “k” in columns 3-6 in the table where k > 1 means the compound is k-fold less potent against the mutant compared to its potency against the wild type, i.e., k = IC₅₀(mutant)/IC₅₀(wild type).
²Compound X is raltegravir (Example 19 in U.S. Pat. No. 7,169,780).
³Compound Y is (—)N-(2-{[(4-fluorobenzyl)amino]-carbonyl}-3-hydroxy-4-oxo-4,6,7,8,9,10-hexahydropyrimido[1,2-a]azepin-10-yl)-N,N′,N′-trimethylethanediamide (Example 12 in U.S. Pat. No. 7,414,045).
⁴Compound Z is N-[(4-fluorophenyl)methyl]-3-hydroxy-9,9-dimethyl-4-oxo-4,6,7,9-tetrahydro-6H-pyrimido[2,1-c][1,4]oxazine-2-carboxamide (compound exemplified in WO 2007/064502 A1).

### Example 34

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention that were tested in the spread assay (see Example 15) were examined for cytotoxicity up to a concentration of 0.5 micromolar, and no cytotoxicity was exhibited. In particular, the compounds set forth in Examples 1 to 30 exhibited no cytotoxicity at concentrations up to 0.5 micromolar.

While the foregoing specification teaches the principles of the present invention, with examples provided for the purpose of illustration, the practice of the invention encompasses all of the usual variations, adaptations and/or modifications that come within the scope of the following claims.

1. A compound of Formula I: 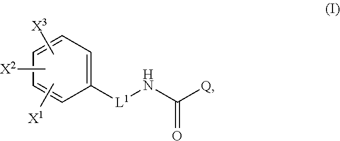
or a pharmaceutically acceptable salt thereof, wherein:
Q is 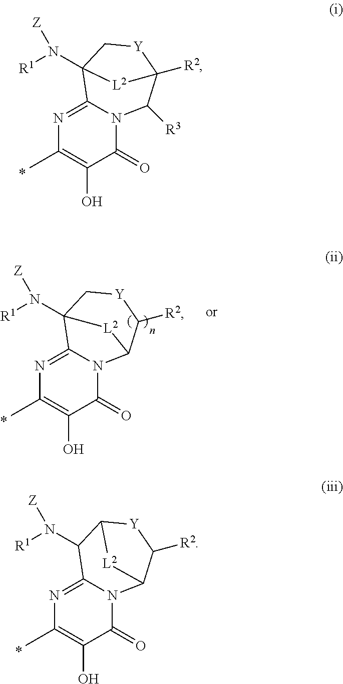
wherein the asterisk * denotes the point of attachment to the rest of the compound;
L¹ is CH₂, CH(CH₃), or C(CH₃)₂;
L² is C₁₋₄ alkylene;
X¹, X² and X³ are each independently selected from the group consisting of:
(1) H,
(2) C₁₋₆ alkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B},
(4) O—C₁₋₆ alkyl,
(5) C₁₋₆ haloalkyl.
(6) O—C₁₋₆ haloalkyl,
(7) OH,
(8) halogen,
(9) CN,
(10) NO₂.
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)N(R^{A})R^{B},
(17) SR^{A},
(18) S(O)R^{A},
(19) SO₂R^{A},
(20) SO₂N(R^{A})R^{B}.
(21) SO₂N(R^{A})C(O)R^{B};
(22) N(R^{A})SO₂R^{B},
(23) N(R^{A})SO₂N(R^{A})R^{B},
(24) N(R^{A})C(O)R^{B},
(25) N(R^{A})C(O)N(R^{A})R^{B},
(26) N(R^{A})C(O)C(O)N(R^{A})R^{B}.
(27) N(R^{A})CO₂R^{B}, and
(28) HetB;
Y is CH₂, CH(CH₃), C(R^{A})(O-AryA), C(R^{A})(OR^{B}), O, S, SO₂, N(R^{A}), or C(O);
Z is:
(1) C(O)N(R^{A})R^{B},
(2) C(O)C(O)N(R^{A})R^{B},
(3) SO₂N(R^{A})R^{B},
(4) C(O)-HetA,
(5) C(O)C(O)-HetA,
(6) SO₂-HetA,
(7) C(O)-HetB.
(8) C(O)C(O)-HetB, or
(9) SO₂-HetB;
R¹ is:
(1) H,
(2) C₁₋₆ alkyl.
(3) C₁₋₆ haloalkyl,
(4) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R³, N(R^{A})C(O)N(R^{A})R^{B}, or N(R^{A})C(O)C(O)N(R^{A})R^{B}, or
(5) C₁₋₆ alkyl substituted with AryC;
R² is:
(1) H,
(2) C₁₋₆ alkyl.
(3) O—C₁₋₆ alkyl,
(4) C₁₋₆ alkyl substituted with O—C₁₋₆ alkyl,
(5) C(O)N(R^{C})R^{D}, or
(6) SO₂N(R^{C})R^{D},
(7) AryB, or
(8) C₁₋₆ alkyl substituted with AryB:
R³ is:
(1) H,
(2) C₁₋₆ alkyl,
(3) C₁₋₆ alkyl substituted with O—C₁₋₆ alkyl,
(4) C(O)N(R^{C})R^{D},
(5) C(O)C(O)N(R^{C})R^{D},
(6) SO₂N(R^{C})R^{D},
(7) AryB, or
(8) C₁₋₆ alkyl substituted with AryB;
n is zero or 1:
each R^{A} is independently H or C₁₋₆ alkyl;
each R^{B} is independently H or C₁₋₆ alkyl;
each R^{C} is independently H or C₁₋₆ alkyl;
each R^{D} is independently H or C₁₋₆ alkyl;
alternatively and independently each pair of R^{C} and R^{D} together with the N atom to which they are both attached form a 4- to 7-membered, saturated or unsaturated, non-aromatic monocyclic ring optionally containing 1 heteroatom in addition to the nitrogen attached to R^{C} and R^{D} selected from N, O, and S, where the S is optionally oxidized to S(O) or S(O)₂; wherein the monocyclic ring is optionally substituted with 1 or 2 substituents each of which is independently:
(1) C₁₋₆ alkyl,
(2) C₁₋₆ haloalkyl,
(3) C₁₋₆ alkyl substituted with OH, O—C₁₋₆ alkyl, O—C₁₋₆ haloalkyl, N(R^{A})R^{B}, C(O)N(R^{A})R^{B}, C(O)R^{A}, CO₂R^{A}, or SO₂R^{A},
(4) O—C₁₋₆ alkyl,
(5) O—C₁₋₆ haloalkyl,
(6) OH,
(7) oxo,
(8) halogen.
(9) C(O)N(R^{A})R^{B},
(10) C(O)R^{A},
(11) C(O)—C₁₋₆ fluoroalkyl,
(12) C(O)OR^{A}, or
(13) S(O)₂R^{A};
AryA is phenyl or naphthyl, wherein the phenyl or naphthyl is optionally substituted with from 1 to 5 substituents each of which is independently any one of the substituents (2) to (28) as set forth above in the definition of X¹, X² and X³;
AryB is phenyl or naphthyl, wherein the phenyl or naphthyl is optionally substituted with from 1 to 5 substituents each of which is independently any one of the substituents (2) to (28) as set forth above in the definition of X¹, X² and X³;
AryC is phenyl or naphthyl, wherein the phenyl or naphthyl is optionally substituted with from 1 to 5 substituents each of which is independently any one of the substituents (2) to (28) as set forth above in the definition of X¹, X² and X³;
HetA is a 4- to 7-membered, saturated or unsaturated, non-aromatic heterocyclic ring containing at least one carbon atom and from 1 to 4 heteroatoms independently selected from N, O and S, where each S is optionally oxidized to S(O) or S(O)₂, wherein the heterocyclic ring is optionally substituted with from 1 to 4 substituents, each of which is independently:
(1) halogen,
(2) C₁₋₆ alkyl,
(3) C₁₋₆ haloalkyl,
(4) O—C₁₋₆ alkyl,
(5) O—C₁₋₆ haloalkyl,
(6) oxo,
(7) C(O)N(R^{A})R^{B},
(8) C(O)C(O)N(R^{A})R^{B},
(9) C(O)R^{A},
(10) CO₂R^{A},
(11) SR^{A},
(12) S(O)R^{A},
(13) SO₂R^{A}, or
(14) SO₂N(R^{A})R^{B}; and
each HetB is independently a 5- or 6-membered heteroaromatic ring containing from 1 to 4 heteroatoms independently selected from N, O and S, wherein the heteroaromatic ring is optionally substituted with from 1 to 4 substituents each of which is independently:
(1) C₁₋₆ alkyl,
(2) C₁₋₆ alkyl substituted with OH, O—C₁₋₆alkyl, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, N(R^{A})C(O)R^{B}, N(R^{A})CO₂R^{B}, N(R^{A})SO₂R^{B}, N(R^{A})SO₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl.
(4) C₁₋₆ haloalkyl,
(5) O—C₁₋₆ haloalkyl.
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)N(R^{A})R^{B},
(16) SR^{A},
(17) S(O)R^{A},
(18) SO₂R^{A},
(19) SO₂N(R^{A})R^{B},
(20) N(R^{A})SO₂R^{B},
(21) N(R^{A})SO₂N(R^{A})R^{B},
(22) N(R^{A})C(O)R^{B}.
(23) N(R^{A})C(O)N(R^{A})R^{B},
(24) N(R^{A})C(O)C(O)N(R^{A})R^{B}, or
(25) N(R^{A})CO₂R^{B}.

2. A compound according to claim 1, or a pharmaceutically acceptable salt thereof, wherein Q is:

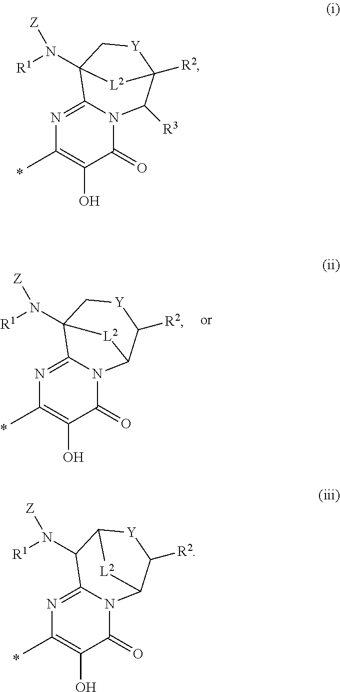

3. A compound according to claim 1, or a pharmaceutically acceptable salt thereof, wherein the compound is a compound of

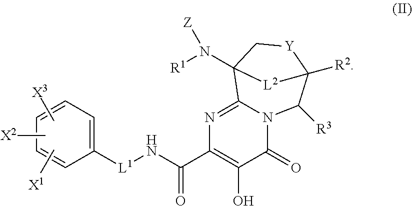

4. A compound according to claim 1, or a pharmaceutically acceptable salt thereof, wherein the compound is a compound of Formula III: 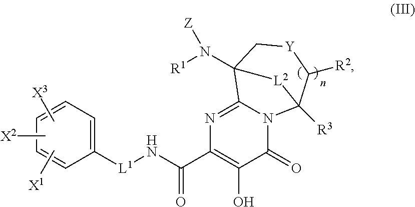
wherein:
L¹ is CH₂;
L² is CH₂ or CH₂CH₂;
X¹ and X² are each independently selected from the group consisting of H, Cl, Br, F, CN, CH₃, CF₃, OH, OCH₃, OCF₃, NH₂, N(H)CH₃, N(CH₃)₂, C(O)NH₉, C(O)N(H)CH₃, C(O)N(CH₃)₂, CH(O), C(O)CH₃, CO₂H, CO₂CH₃, SO₂H and SO₂CH₃; and provided that
(i) at least one of X¹ and X² is other than 11;
(ii) X¹ is in the para position on the phenyl ring; and
(iii) X² is in the meta position on the phenyl ring;
X³ is H;
Y is CH₂ or O;
Z is C(O)N(CH₃)₂, C(O)C(O)NH(CH₃), C(O)C(O)N(CH₃)₂, 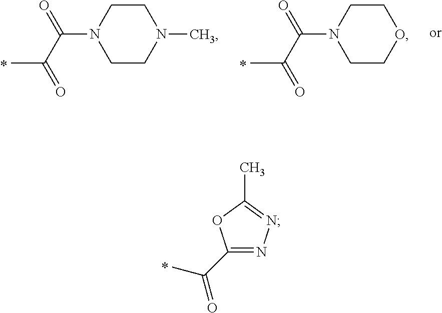
R¹ is H, CH₃, CH₂CH₃, or CH₂CH₂CH₃; and
R² is H, CH₃, CH₂CH₃, OCH₃ or OH.

5. (canceled)

6. A compound according to claim 1, or a pharmaceutically acceptable salt thereof, wherein the compound is a compound of Formula IV:

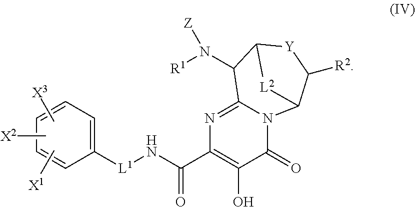

7. A compound according to claim 1, or a pharmaceutically acceptable salt thereof, wherein:
L¹ is CH₂;
L² is CH₂, C(CH₃), C(CH₃)₂, CH₂CH₂, or CH₂CH₂Cl₂;
X¹, X² and X³ are each independently selected from the group consisting of H, halogen, CN, NO₂, C₁₋₄ alkyl, C₁₋₄ haloalkyl, OH, O—C₁₋₄ alkyl, O—C₁₋₄ haloalkyl. N(R^{A})R^{B}, C(O)N(R^{A})R^{B}, C(O)R^{A}, CO₂R^{A}, SR^{A}, S(O)R^{A}, SO₂R^{A}, SO₂N(R^{A})R^{B}, SO₂N(R^{A})C(O)R^{B}, N(R^{A})SO₂R^{B}, N(R^{A})SO₂N(R^{A})R^{B}, N(R^{A})C(O)R^{B}, and N(R^{A})C(O)C(O)N(R^{A})R^{B}; and provided that at least one of X¹, X² and X³ is other than H;
Y is CH₂ or O;
Z is:
(1) C(O)N(R^{A})R^{B},
(2) C(O)C(O)N(R^{A})R^{B},
(3) C(O)-HetA,
(4) C(O)C(O)-HetA,
(5) C(O)-HetB, or
(6) C(O)C(O)-HetB;
R¹ is H or C₁₋₄ alkyl;
R² is:
(1) H,
(2) —C₁₋₄ alkyl,
(3) O—C₁₋₄ alkyl,
(4) C₁₋₄ alkyl substituted with O—C₁₋₆ alkyl,
(5) C(O)N(R^{C})R^{D},
(6) SO₂N(R^{C})R^{D},
(7) AryB, or
(8) C₁₋₄ alkyl substituted with AryB;
R³ is:
(1) H,
(2) C₁₋₄ alkyl,
(3) C₁₋₄ alkyl substituted with O—C₁₋₄ alkyl,
(4) C(O)N(R^{C})R^{D}.
(5) C(O)C(O)N(R^{C})R^{D},
(6) SO₂N(R^{C})R^{D}.
(7) AryB, or
(8) C₁₋₄ alkyl substituted with AryB:
each R^{A} is independently H or C₁₋₄ alkyl;
each R^{B} is independently H or C₁₋₄ alkyl;
each R^{C} is independently H or C₁₋₄ alkyl;
each R^{D} is independently H or C₁₋₄ alkyl;
alternatively and independently each pair of R^{C} and R^{D} together with the N atom to which they are both attached form a 4- to 7-membered, saturated monocyclic ring optionally containing 1 heteroatom in addition to the nitrogen attached to R^{C} and R^{D} selected from N, O, and S, where the S is optionally oxidized to S(O) or S(O)₂; wherein the monocyclic ring is optionally substituted with 1 or 2 substituents each of which is independently:
(1) C₁₋₄ alkyl.
(2) C₁₋₄ fluoroalkyl,
(3) O—C₁₋₄ alkyl,
(4) O—C₁₋₄ fluoroalkyl,
(5) oxo,
(6) C(O)R^{A},
(7) CO₂R^{A}, or
(8) SO₂R^{A};
AryB is phenyl optionally substituted with from 1 to 3 substituents each of which is independently:
(1) C₁₋₄ alkyl.
(2) OH,
(3) O—C₁₋₄ alkyl,
(4) C₁₋₄ haloalkyl,
(5) O—C₁₋₄ haloalkyl,
(6) halogen,
(7) CN,
(8) N(R^{A})R^{B},
(9) C(O)N(R^{A})R^{B},
(10) C(O)R^{A},
(11) C(O)OR^{A},
(12) SR^{A},
(13) S(O)R^{A},
(14) SO₂R^{A},
(15) SO₂N(R^{A})R^{B},
(16) SO₂N(R^{A})C(O)R^{B},
(17) N(R^{A})SO₂R^{B},
(18) N(R^{A})SO₂N(R^{A})R^{B},
(19) N(R^{A})C(O)R^{B}, or
(20) N(R^{A})C(O)C(O)N(R^{A})R^{B};
HetA is a 4- to 7-membered, saturated heterocyclic ring containing an N atom and optionally containing an additional heteroatom selected from N, O and S, wherein (i) the heterocyclic ring is attached to the C(O) moiety via an N atom, (ii) the optional S atom is optionally oxidized to S(O) or S(O)₂, and (iii) the heterocyclic ring is optionally substituted with from 1 to 3 substituents, each of which is independently:
(1) C₁₋₄ alkyl,
(2) C₁₋₄ fluoroalkyl,
(3) O—C₁₋₄ alkyl,
(4) O—C₁₋₄ fluoroalkyl,
(5) oxo,
(6) C(O)R^{A},
(7) CO₂R^{A}, or
(8) SO₂R^{A}; and
HetB is a 5- or 6-membered heteroaromatic ring containing a total of from 1 to 4 heteroatoms independently selected from 1 to 4 N atoms, zero or 1 O atom, and zero or 1 S atom, wherein the heteroaromatic ring is optionally substituted with from 1 to 3 substituents each of which is independently:
(1) C₁₋₄ alkyl,
(2) C₁₋₄ fluoroalkyl,
(3) O—C₁₋₄ alkyl,
(4) O—C₁₋₄ fluoroalkyl.
(5) OH,
(6) C(O)R^{A},
(7) CO₂R^{A}, or
(8) SO₂R^{A}.

8. A compound according to claim 7, or a pharmaceutically acceptable salt thereof, wherein:
X¹ and X² are each independently selected from the group consisting of
H, Cl, Br, F, CN, CH₃, CF₃, OH, OCH₃, OCF₃, NH₂, N(H)CH₃, N(CH₃)₂, C(O)NH₂, C(O)N(H)CH₃, C(O)N(CH₃)₂, CH(O), C(O)CH₃, CO₂H, CO₂CH₃, SO₂H and SO₂CH₃; and provided that at least one of X¹ and X² is other than H;
X³ is H;
Z is C(O)N(CH₃)₂, C(O)C(O)NH(CH₃), C(O)C(O)N(CH₃)₂, 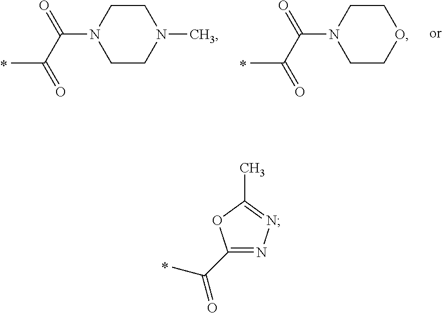
R¹ is CH₃, CH₂CH₃, CH₂CH₂CH₃, or CH(CH₃)₂;
R² is H, CH₃, CH₂CH₃, OCH₃, CH₂OCH₃, phenyl, or benzyl; wherein the phenyl or the phenyl moiety in benzyl is optionally substituted with 1 or 2 substituents each of which is independently Cl, Br, F, CH₃, CF₃, OCH₃, OCF₃, C(O)NH₂, C(O)N(H)CH₃, C(O)N(CH₃)₂, C(O)CH₃, CO₂CH₃, or SO₂CH₃;
R³ is H, CH₃, CH₂CH₃, phenyl, or benzyl; wherein the phenyl or the phenyl moiety in benzyl is optionally substituted with 1 or 2 substituents each of which is independently Cl, Br, F, CH₃, CF₃, OCH₃, OCF₃, CN, C(O)NH₂, C(O)N(H)CH₃, C(O)N(CH₃)₂, C(O)CH₃, CO₂CH₃, or SO₂CH₃;
AryB is phenyl optionally substituted with from 1 to 3 substituents each of which is independently:
(1) C₁₋₃ alkyl,
(2) O—C₁₋₃ alkyl,
(3) CF₃,
(4) OCF₃,
(5) Cl,
(6) Br,
(7) F,
(8) CN,
(9) C(O)NH₂,
(10) C(O))N(H)—C₁₋₃ alkyl,
(11) C(O)N(—C₁₋₃ alkyl)₂,
(12) C(O)—C₁₋₃ alkyl,
(13) C(O)O—C₁₋₃ alkyl, or
(14) SO₂—C₁₋₃ alkyl;
HetA is a saturated heterocyclic ring selected from the group consisting of: 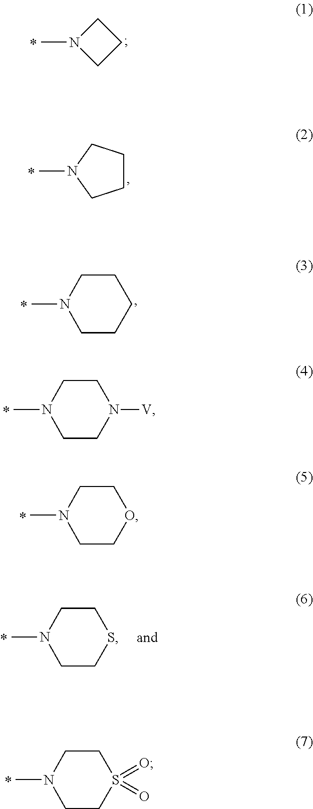
each V is independently H, C₁₋₃ alkyl, C(O)—C₁₋₃ alkyl, C(O)—O—C₁₋₃ alkyl, or S(O)₂—C₁₋₃ alkyl; and
HetB is a heteroaromatic ring selected from the group consisting of pyrrolyl, pyrazolyl, imidazolyl, pyridinyl, pyrimidinyl, pyrazinyl, oxazolyl, isoxazolyl, thiazolyl, isothiazolyl, oxadiazolyl, and thiadiazolyl, wherein the heteroaromatic ring is optionally substituted with from 1 to 2 substituents each of which is independently a C₁₋₄ alkyl.

9. (canceled)

10. A compound according to claim 3, or a pharmaceutically acceptable salt thereof, wherein:
L¹ is CH₂;
L² is CH₂ or CH₂CH₂;
X¹ and X² are each independently selected from the group consisting of H, Cl, Br, F, CN, CH₃, CF₃, OH, OCH₃, OCF₃, NH₂, N(H)CH₃, N(CH₃)₂, C(O)NH₂. C(O)N(H)CH₃, C(O)N(CH₃)₂, CH(O), C(O)CH₃, CO₂H, CO₂CH₃, SO₂H and SO₂CH₃; and provided that
(i) at least one of X¹ and X² is other than H;
(ii) X¹ is in the para position on the phenyl ring; and
(iii) X² is in the meta position on the phenyl ring;
X³ is H:
Y is CH₂ or O;
Z is C(O)N(CH₃)₂, C(O)C(O)NH(CH₃), C(O)C(O)N(CH₃)₂, 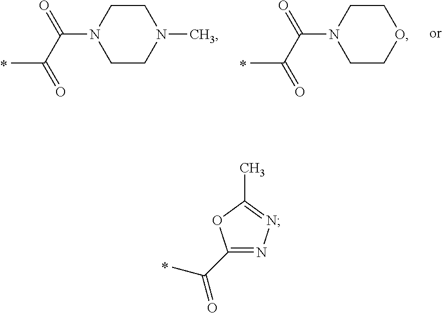
R¹ is H, CH₃, CH₂CH₃, or CH₂CH₂CH₃;
R² is H, CH₃, CH₂CH₃, OCH₃ or OH; and
R³ is H, CH₃, or CH₂CH₃.

11. (canceled)

12. (canceled)

13. (canceled)

14. A compound according to claim 7, or a pharmaceutically acceptable salt thereof, wherein the compound is a compound of Formula V-B:

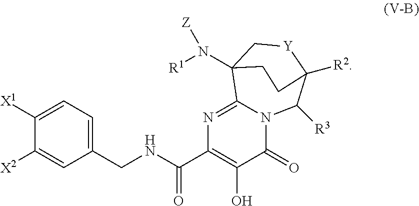

15. A compound according to claim 14, or a pharmaceutically acceptable salt thereof, wherein X¹ is F; and X² is H or CH₃.

16. (canceled)

17. A compound according to claim 4, or a pharmaceutically acceptable salt thereof, wherein X¹ is F; and X² is H or CH₃.

18. (canceled)

19. (canceled)

20. A compound according to claim 7, or a pharmaceutically acceptable salt thereof, wherein the compound is a compound of Formula VI-A: 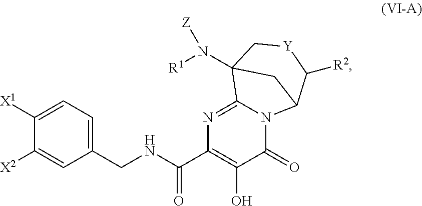
wherein X¹ is F; and X² is H or CH₃

21. (canceled)

22. A compound according to claim 7, or a pharmaceutically acceptable salt thereof, wherein the compound is a compound of Formula VI-B:

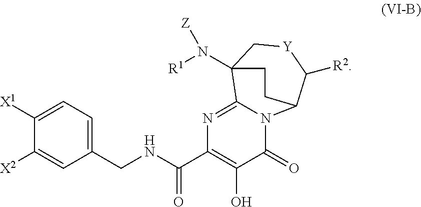

23. A compound according to claim 22, or a pharmaceutically acceptable salt thereof, wherein X¹ is F; and X² is H or CH₃.

24. A compound according to claim 7, or a pharmaceutically acceptable salt thereof, wherein the compound is a compound of Formula VI-C: 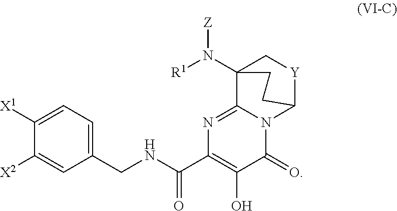
wherein X¹ is F; and X² is H or CH₃.

25. (canceled)

26. A compound according to claim 1, or a pharmaceutically acceptable salt thereof, selected from the group consisting of:
N-(4-{[(4-fluorobenzyl)amino]carbonyl}-5-hydroxy-6-oxo-3,7-diazatricyclo[7.2.2.0^{2,7}]trideca-2,4-dien-1-yl)-N′,N′,N″-trimethylethanediamide;
N-(4-{[(4-fluoro-3-methyl benzyl)amino]carbonyl}-5-hydroxy-6-oxo-3,7-diazatricyclo[7.2.2.0^{2,7}]trideca-2,4-dien-1-yl)-N′,N′,N″-trimethylethanediamide;
N-(4-fluorobenzyl)-5-hydroxy-1-{methyl[morpholin-4-yl(oxo)acetyl]amino}-6-oxo-3,7-diazatricyclo[7.2.2.0^{2,7}]trideca-2,4-diene-4-carboxamide;
N-(4-fluorobenzyl)-5-hydroxy-1-{{methyl[(4-methylpiperazin-1-yl)(oxo)acetyl]amino}-6-oxo-3,7-diazatricyclo[7.2.2.0^{2,7}]trideca-2,4-diene-4-carboxamide;
N′-{2-[(4-fluorobenzyl)carbamoyl]-3-hydroxy-4-oxo-6,7,8,9-tetrahydro-7,10-ethanopyrimido[1,2-a]azepin-10(4H)-yl}-N,N-dimethylethanediamide;
N-(4-fluorobenzyl)-3-hydroxy-10-{[morpholin-4-yl(oxo)acetyl]amino}-4-oxo-4,6,7,8,9,10-hexahydro-7,10-ethanopyrimido[1,2-a]azepine-2-carboxamide;
N-{2-[(4-fluorobenzyl)carbamoyl]-3-hydroxy-4-oxo-6,7,8,9-tetrahydro-7,10-methanopyrimido[1,2-a]azepin-10(4H)-yl}-N,N′,N′-trimethylethanediamide;
N-{2-[(4-Fluorobenzyl)carbamoyl]-3-hydroxy-4-oxo-6,7,8,9-tetrahydro-7,10-methanopyrimido[1,2-a]azepin-10(4H)-yl}-N,N′,N′-trimethylethanediamide;
N-(4-Fluorobenzyl)-3-hydroxy-10-{methyl[morpholin-4-yl(oxo)acetyl]amino}-4-oxo-4,6,7,8,9,10-hexahydro-7,10-methanopyrimido[1,2-a]azepine-2-carboxamidel;
(+)-N-(-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′N′-trimethylethanediamide;
(−)-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′,N′-trimethylethanediamide;
(+/−)-N-4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′,N′-trimethylethanediamide;
(+)-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′,N′-trimethylethanediamide;
(−)-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′,N′-trimethylethanediamide;
(+/−)-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′,N′-trimethylethanediamide;
(+)-N-ethyl-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′-dimethylethanediamide;
(−)-N-ethyl-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)N′,N′-dimethylethanediamide;
(+/−)-N-ethyl-N-(4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N′,N′,N″-dimethylethanediamide;
(+)-N-(4-{[(4-fluorobenzyl)amino]carbonyl}-5-hydroxy-8-methyl-6-oxo-3,7-diazatricyclo[7.2.2.0^{2,7}]trideca-2,4-dien-1-yl)-N′,N′,N″-trimethylethanediamide;
(−)-N-(4-{[(4-fluorobenzyl)amino]carbony}-5-hydroxy-8-methyl-6-oxo-3,7-diazatricyclo[7.2.2.0^{2,7}]trideca-2,4-dien-1-yl)N′,N′,N″-trimethylethanediamide; and
(+/−)-N-(4-{[(4-fluorobenzyl)amino]carbonyl}-5-hydroxy-8-methyl-6-oxo-3,7-diazatricyclo[7.2.2.0^{2,7}]trideca-2,4-dien-1-yl)-N,N′,N″-trimethylethanediamide.

27. A compound according to claim 1, or a pharmaceutically acceptable salt thereof, selected from the group consisting of:
N-(4-{[(4-fluoro-3-methylbenzyl)amino]carbonyl}-5-hydroxy-9-methoxy-6-oxo-3,7-diazatricyclo[7.2.2.0^{2,7}]trideca-2,4-dien-1-yl)-N,N′,N′-trimethylethanediamide;
N-(4-{[(4-fluorobenzyl)amino]carbonyl}-5-hydroxy-9-methoxy-6-oxo-3,7-diazatricyclo[7.2.2.0^{2,7}]trideca-2,4-dien-1-yl)-N,N′,N′-trimethylethanediamide;
N-ethyl-N-(4-{[(4-fluorobenzyl)amino]carbonyl}-5-hydroxy-9-methoxy-6-oxo-3,7-diazatricyclo[7.2.2.0^{2,7}]trideca-2,4-dien-1-yl)-N′,N′-dimethylethanediamide;
N-(4-{[(4-Fluorobenzyl)amino]carbonyl}-5,9-dihydroxy-6-oxo-3,7-diazatricyclo[7.2.2.0^{2,7}]trideca-2,4-dien-1-yl)-N,N′,N′-trimethylethanediamide;
N-ethyl-N-(4-{[(4-fluoro-3-methylbenzyl)amino]carbonyl}-5-hydroxy-6-oxo-10-oxa-3,7-diazatricyclo[7.2.2.0^{2,7}]trideca-2,4-dien-1-yl)-N′,N′-dimethylethanediamide;
N-(4-{[(4-fluorolbenzyl)amino]carbonyl}-5-hydroxy-6-oxo-10-oxa-3,7-diazatricyclo[7.2.2.0^{2,7}]trideca-2,4-dien-1-yl)-N′,N′-dimethyl-N-propylethanediamide;
N-(9-ethyl-4-{[(4-fluorolbenzyl)amino]carbonyl}-5-hydroxy-6-oxo-10-oxa-3,7-diazatricyclo[7.2.2.0^{2,7}]trideca-2,4-dien-1-yl)-N,N′,N′-trimethylethanediamide;
N-4-{[(4-fluorolbenzyl)amino]carbonyl}-5-hydroxy-9-methyl-6-oxo-10-oxa-3,7-diazatricyclo[7.2.2.0^{2,7}]trideca-2,4-dien-1-yl)-N,N′,N′-trimethylethanediamide;
N′-(9-ethyl-4-{[(4-fluorolbenzyl)amino]carbonyl}-5-hydroxy-6-oxy-10-oxa-3,7-diazatricyclo[7.2.2.0^{2,7}]trideca-2,4-dien-1-yl)-N,N-dimethylethanediamide;
N-5-{[(4-fluorolbenzyl)amino]carbonyl}-4-hydroxy-3-oxo-10-oxa-2,6-diazatricyclo[6.3.2.0^{2,7}]trideca-4,6-dien-8-yl)-N,N′,N′-trimethylethanediamide;
N-5-{[(4-fluorolbenzyl)amino]carbonyl}-4-hydroxy-3-oxo-2,6-diazatricyclo[6.3.2.0^{2,7}]trideca-4,6-dien-8-yl)-N,N′,N′-trimethylethanediamide;
N-(8-ethyl-4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N,N′,N′-trimethylethanediamide;
N′-(8-ethyl-4-{[(4-fluorobenzyl)amino]carbonyl}-5-hydroxy-6-oxo-10-oxa-3,7-diazatricyclo[7.2.2.0^{2,7}]trideca-2,4-dien-1-yl)-N,N′,N′-dimethylethanediamine;
N-(4-{[(4-fluorobenzyl)amino]carbonyl}-5-hydroxy-8-methyl-6-oxo-10-oxa-3,7-diazatricyclo[7.2.2.0^{2,7}]trideca-2,4-dien-1-yl)-N,N′,N′-trimethylethanediamide;
N-(4-{[(4-fluorobenzyl)amino]carbonyl}-5-hydroxy-8-methyl-6-oxo-10-oxa-3,7-diazatricyclo[7.2.2.0^{2,7}]trideca-2,4-dien-1-yl)-N,N′,N′-dimethylethanediamide;
N′-(4-{[(4-fluorobenzyl)amino]carbonyl}-5-hydroxy-8-methyl-6-oxo-10-oxa-3,7-diazatricyclo[7.2.2.0^{2,7}]trideca-2,4-dien-1-yl)-N-dimethylethanediamide; and
N-5-{[(4-fluorolbenzyl)amino]carbonyl}-4-hydroxy-3-oxo-2,6-diazatricyclo[6.2.2.0^{2,7}]dodeca-4,6-dien-8-yl)-N,N′,N′-trimethylethanediamide.

28. A pharmaceutical composition comprising an effective amount of a compound according to claim 1, or a pharmaceutically acceptable salt thereof, and a pharmaceutically acceptable carrier.

29. A method for the treatment or prophylaxis of infection by HIV or for the treatment, prophylaxis, or delay in the onset or progression of AIDS in a subject in need thereof, which comprises administering to the subject an effective amount of the compound according to claim 1 or a pharmaceutically acceptable salt thereof.

30. The method according to claim 29, wherein the HIV is HIV-1.

31. (canceled)

32. (canceled)
